(19) United States
(12) Patent Application Publication
(10) Pub. No.: US 2006/0147447 A1
(43) Pub. Date: Jul. 6, 2006

(54) Secreted peptides

(75) Inventors: Lydie Bougueleret, Meyrin/GE (CH); Anne Niknejad, Meyrin/GE (CH)

Correspondence Address: NOVARTIS;CORPORATE INTELLECTUAL PROPERTY, ONE HEALTH PLAZA 104/3, EAST HANOVER, NJ, 07936-1080, US

(73) Assignee: GENOVA Ltd., Hamilton HM 12 (BM)

(21) Appl. No.: 10/519,328

(22) PCT Filed: Jul. 2, 2003
(86) PCT No.: PCT/EP03/07069

§ 371 Date: Oct. 7, 2005

Related U.S. Application Data
(60) Provisional application No. 60/393,840, filed on Jul. 2, 2002.

(51) Int. Cl. A61K 38/17; C07K 14/705; C07K 16/28; A61K 39/395

(52) U.S. Cl. 424/143.1; 530/350; 530/388.22; 514/12

(57) ABSTRACT

The invention provides secreted active peptides, methods and compositions for making such peptides, and methods of using the peptides in detection assays, for disease diagnosis, for disease treatment and for drug development.

### FIELD OF THE INVENTION

The invention relates to secreted active peptides, isolated polynucleotides encoding such peptides, polymorphic variants thereof, and the use of said nucleic acids and peptides or compositions thereof in detection assays, for disease diagnosis, for disease treatment and for drug development.

### BACKGROUND

LTBPs are a family of Latent Transforming Growth Factor (TGF)-beta Binding Proteins that associate with TGF-beta to regulate secretion and the activation of the growth factor. Three different members of the LTBP family have been identified so far (Oklu R. and Hesketh R., Biochem. journal., 325; 601-610, (2000)).

LTBP-2 has a multidomain structure containing cysteine-rich motifs that are also found in the fibrillin gene family. LTBP-2 primarily acts as a structural component of microfibrils and elastic fibers during development and wound healing (Bashir M M, et al., The International Journal of Biochemistry & Cell Biology, 28:531-42, (1996) and Shipley J M, et al., Molecular and Cellular Biology, July; 20(13):4879-87, (2000)). The N terminal region of LTBP-2 is proline rich and is targeted by extracellular matrix-associated proteases such as elastase and plasmin (Hyytiainen M, et al., J Biol Chem 1998; 273(32):20669-76).

### SUMMARY OF THE INVENTION

The present invention is directed to compositions related to secreted active peptides, designated herein “Pep356-related” peptides. Such compositions include Pep356-related peptides, having an amino acid sequence of SEQ ID NO: 3 through 5, Pep356-related peptide precursors, Pep356-related antibodies, including monoclonal antibodies and other binding compositions derived therefrom, and methods of making and using these compositions. Pep356-related peptides precursors of the invention include the Pep356-related peptide precursors of SEQ ID NO: 1 and 2.

In another aspect, the invention includes polypeptides comprising a sequence at least 75 percent identical to a sequence selected from SEQ ID NOs: 3-5. Preferably, the invention includes polypeptides having at least 80 percent, and more preferably at least 85 percent, and still more preferably at least 90 percent, identity with any one of the sequences selected from SEQ ID NOs: 3-5. Most preferably, the invention includes polypeptides having a sequence at least 95 percent identical to a sequence selected from the group consisting of SEQ ID NOs: 3-5. Even more preferably, the invention includes polypeptides having a sequence at least 98 percent identical to a sequence selected from the group consisting of SEQ ID NOs: 3-5

A preferred embodiment of the invention includes Pep356-related peptides having a posttranslational modification, such as an amidation, phosphorylation, glycosylation, acetylation, or an N- or O-linked carbohydrate group. Additionally preferred are Pep356-related peptides with intra- or inter-molecular interactions, e.g., disulfide and hydrogen bonds, that result in higher order structures. Also preferred are Pep356-related peptides that result from differential mRNA processing or splicing.

In an additional aspect, the invention includes modified Pep356-related peptides. Such modifications include protecting/blocking groups, linkage to an antibody molecule or other cellular ligand, and detectable labels, such as an enzymatic, fluorescent, isotopic or affinity label to allow for detection and isolation of the protein. Chemical modifications may be carried out by known techniques, including but not limited, to specific chemical cleavage by cyanogen bromide, trypsin, chymotrypsin, papain, V8 protease, NaBH4, acetylation, formylation, oxidation, reduction, or metabolic synthesis in the presence of tunicamycin. Also provided by the invention are chemically modified derivatives of the polypeptides of the invention which may provide additional advantages such as increased solubility, stability and circulating time of the polypeptide, or decreased immunogenicity (e.g., water soluble polymers such as polyethylene glycol, ethylene glycol/propylene glycol copolymers, carboxymethylcellulose, dextran, polyvinyl alcohol). Pep356-related peptides are modified at random positions within the molecule, or at predetermined positions within the molecule and may include one, two, three or more attached chemical moieties.

A preferred aspect of the invention provides a composition comprising an isolated Pep356-related peptide, i.e., a Pep356-related peptide free from proteins or protein isoforms having a significantly different isoelectric point or a significantly different apparent molecular weight from the Pep356-related peptide. The isoelectric point and molecular weight of a Pep356-related peptide may be indicated by affinity and size-based separation chromatography, 2-dimentional gel analysis, and mass spectrometry.

In a preferred aspect, the invention provides particular polypeptide species that comprise an amino acid sequence selected from the group consisting of SEQ ID NOs:3-5. Preferably, the particular polypeptide species further comprises contiguous amino acid sequence from SEQ ID NO: 1. Preferred species are polypeptides that i) comprise an amino acid sequence selected from the group consisting of SEQ ID NOs:3-5; ii) appear in human blood plasma; and iii) result from proteolytic processing of the polypeptide of SEQ ID NO: 1.

In another aspect, the invention includes polynucleotides coding for a polypeptide having an amino acid sequence selected from the group consisting of SEQ ID NO: 3 through 5, antisense oligonucleotides complementary to such sequences, oligonucleotides complementary to Pep356 gene sequences useful in diagnostic and analytical assays, such as primers for polymerase chain reactions (PCRs), and vectors for expressing Pep356-related peptides.

In another aspect, the invention includes primer pairs for carrying out a PCR to amplify a segment of a polynucleotide of the invention. Each primer of a pair is an oligonucleotide having a length of between 15 and 30 nucleotides such that i) one primer of the pair forms a perfectly matched duplex with one strand of a polynucleotide of the invention and the other primer of the pair forms a perfectly matched duplex with the complementary strand of the same polynucleotide, and ii) the primers of a pair form such perfectly matched duplexes at sites on the polynucleotide that are separated by a distance of between 10 and 2500 nucleotides. Preferably, the annealing temperature of each primer of a pair with its respective complementary sequence is substantially the same.

The invention further includes methods of using Pep356-related compositions, including antisense and antibody compounds, to treat disorders associated with aberrant expression of the Pep356-related peptides of SEQ ID NO: 3 through 5 in an individual, and methods of using Pep356-related compositions, including primers complementary to the Pep356 gene and/or messenger RNA and anti-Pep356-related antibodies, for detecting and measuring quantities of the Pep356-related peptides in tissues and biological fluids. The invention also further includes methods of screening for compounds that inhibit or increase the expression of Pep356-related peptides, as well as methods of screening for compounds that interact with and/or inhibit or increase the activity of Pep356-related peptides. The invention further encompasses compounds thus identified, as well as compositions thereof.

In another aspect, the invention includes isolated antibodies specific for any of the polypeptides. peptide fragments, or peptides described above. Preferably, the antibodies of the invention are monoclonal antibodies. Further preferred are antibodies that bind to a Pep356-related peptide exclusively, that is, antibodies that do not recognize other polypeptides with high affinity. Anti-Pep356-related peptide antibodies have purification, diagnostic and therapeutic applications, particularly in with respect to Pep356-related peptide-related disorders. Preferred anti- Pep356-related peptide antibodies for purification and diagnosis are attached to a label group. Treatment methods include, but are not limited to, those that employ antibodies or antibody-derived compositions specific for a Pep356-related antigen. Diagnostic methods for detecting Pep356-related peptides in specific tissue samples, and for detecting levels of expression of Pep356-related peptides in tissues, also form part of the invention. Compositions comprising one or more antibodies described above, together with a pharmaceutically acceptable carrier are also within the scope of the invention.

In another aspect, the invention includes natural variants of the Pep356-related peptide having a frequency in a selected population of at least two percent. More preferably, such natural variant has a frequency in a selected population of at least five percent, and still more preferably, at least ten percent. Most preferably, such natural variant has a frequency in a selected population of at least twenty percent. The selected population may be any recognized population of study in the field of population genetics. Preferably, the selected population is Caucasian, Negroid, or Asian. More preferably, the selected population is French, German, English, Spanish, Swiss, Japanese, Chinese, Irish, Korean, Singaporean, Icelandic, North American, Israeli, Arab, Turkish, Greek, Italian, Polish, Pacific Islander, Finnish, Norwegian, Swedish, Estonian, Austrian, or Indian. More preferably, the selected population is Icelandic, Saarni, Finnish, French of Caucasian ancestry, Swiss, Singaporean of Chinese ancestry, Korean, Japanese, Quebecian, North American Pima Indians, Pennsylvanian Amish and Amish Mennonite, Newfoundlander, or Polynesian. In another aspect, the invention provides a vector comprising DNA encoding a Pep356-related peptide. The invention also includes host cells and transgenic non human animals comprising such a vector. There is also provided a method of making a Pep356-related peptides or Pep356-related peptides precursor. The method comprises the steps of (a) providing a host cell containing an expression vector as disclosed above; (b) culturing the host cell under conditions whereby the DNA segment is expressed; and (c) recovering the protein encoded by the DNA segment. Another preferred method comprises the steps of: (a) providing a host cell capable of expressing a Pep356-related peptide; (b) culturing said host cell under conditions that allow expression of said Pep356-related peptide; and (c) recovering said Pep356-related peptide. Within one embodiment the expression vector further comprises a secretory signal sequence operably linked to the DNA segment, the cell secretes the protein into a culture medium, and the protein is recovered from the medium. An especially preferred method of making a Pep356-related peptide includes chemical synthesis using standard peptide synthesis techniques, as described in the section titled “Chemical Manufacture of Pep356-related compositions” and in Example 2.

In still a further aspect, the invention includes pharmaceutical compositions and formulations comprising a polypeptide having an amino acid sequence selected from the group consisting of SEQ ID NO: 3 through 5, and a pharmaceutically acceptable carrier compound.

Further aspects of the invention are also described in the specification and in the claims.

BRIEF DESCRIPTION OF THE SEQUENCE LISTING

| SEQ ID | content |
|---|---|
| 1 | protein precursor |
| 2 | mature protein |
| 3 | active peptide 1 |
| 4 | active peptide 2 |
| 5 | active peptide 3 |

### BRIEF DESCRIPTION OF THE FIGURES

FIG. 1 shows the sequence of one Pep356-related peptide of the invention, from SEQ ID NO 3. The tryptic peptides observed by tandem mass spectrometry are bold and underlined. The tryptic peptides observed by MALDI only are in italics and underlined. Finally, the tryptic peptides observed by tandem and MALDI mass spectrometry are bold, in italics, and doubly underlined. The three dibasic cleavage sites between residues 61 and 79 are shaded.

FIG. 2 illustrates the N terminal divergence of known human LTBPs. The signal peptides are shaded. The proteins are represented as follows: LTBL_HUMAN: Latent transforming growth factor beta binding protein, isoform IL; LTBS_HUMAN: Latent transforming growth factor beta binding protein, isoform IS; Q9NS 15: Latent transforming growth factor beta binding protein 3; Q14767: Latent transforming growth factor-beta-binding protein-2 (SEQ ID NO 1); 075412: Latent transforming growth factor-beta binding protein 4S.

FIG. 3 illustrates the interspecies conservation of the LTBP2 protein, particularly at its N-terminus. The species are represented as follows: Q14767: human (SEQ ID NO 1); Q28019: Bos Taurus; O08999: Mus musculus; and O35806: Rattus Norvegicus. The signal peptides are shaded and the three dibasic cleavage sites are bold and underlined.

### DETAILED DESCRIPTION OF THE INVENTION

The present invention described in detail below provides methods, compositions, and kits useful for screening, diagnosis, and treatment of a Pep356-related peptide disorder in a mammalian individual; for identifying individuals most likely to respond to a particular therapeutic treatment; for screening Pep356-related peptide modulators; and for drug development. When the invention is used for drug development, e.g., to characterize a Pep356-related modulator, the body fluid analyzed for the level of at least one Pep356-related peptide is preferably from a non-human mammal. The nonhuman mammal is preferably one in which the induction of a TGF-beta response by endogenous and/or exogenous agents is predictive of the induction of such a response in a human. Rodents and primates are particularly suitable. For clarity of disclosure, and not by way of limitation, the invention will be described with respect to the analysis of blood plasma samples. However, as one skilled in the art will appreciate, the assays and techniques described below can be applied to other biological fluid samples (e.g. cerebrospinal fluid, lymph, bile, serum, saliva or urine) or tissue samples from an individual at risk of having or developing a Pep356-related peptide disorder. The methods and compositions of the present invention are useful for screening, diagnosis and prognosis of a living individual, but may also be used for postmortem diagnosis in an individual, for example, to identify family members who are at risk of developing the same disorder.

Definitions

As used herein, the term “nucleic acids” and “nucleic acid molecule” is intended to include DNA molecules (e.g., cDNA or genomic DNA) and RNA molecules (e.g., mRNA) and analogs of the DNA or RNA generated using nucleotide analogs. The nucleic acid molecule can be single-stranded or double-stranded, but preferably is double-stranded DNA. Throughout the present specification, the expression “nucleotide sequence” may be employed to designate indifferently a polynucleotide or a nucleic acid. More precisely, the expression “nucleotide sequence” encompasses the nucleic material itself and is thus not restricted to the sequence information (i.e. the succession of letters chosen among the four base letters) that biochemically characterizes a specific DNA or RNA molecule. Also, used interchangeably herein are terms “nucleic acids”, “oligonucleotides”, and “polynucleotides”.

An “isolated” nucleic acid molecule is one which is separated from other nucleic acid molecules which are present in the natural source of the nucleic acid. Preferably, an “isolated” nucleic acid is free of sequences which naturally flank the nucleic acid (i.e., sequences located at the 5′ and 3′ ends of the nucleic acid) in the genomic DNA of the organism from which the nucleic acid is derived. For example, in various embodiments, the isolated Pep356-related nucleic acid molecule can contain less than about 5 kb, 4 kb, 3 kb, 2 kb, 1 kb, 0.5 kb or 0.1 kb of nucleotide sequences which naturally flank the nucleic acid molecule in genomic DNA of the cell from which the nucleic acid is derived. Moreover, an “isolated” nucleic acid molecule, such as a cDNA molecule, can be substantially free of other cellular material, or culture medium when produced by recombinant techniques, or substantially free of chemical precursors or other chemicals when chemically synthesized. A nucleic acid molecule of the present invention can be isolated using standard molecular biology techniques and the sequence information provided herein. Using all or a portion of the nucleic acid, as a hybridization probe, Pep356-related nucleic acid molecules can be isolated using standard hybridization and cloning techniques (e.g., as described in Sambrook, J., Fritsh, E. F., and Maniatis, T. Molecular Cloning. A Laboratory Manual. 2nd, ed., Cold Spring Harbor Laboratory, Cold Spring Harbor Laboratory Press, Cold Spring Harbor, N.Y., 1989).

A nucleic acid of the invention can be amplified using cDNA, mRNA or alternatively, genomic DNA, as a template and appropriate oligonucleotide primers according to standard PCR amplification techniques. The nucleic acid so amplified can be cloned into an appropriate vector and characterized by DNA sequence analysis. Furthermore, oligonucleotides corresponding to Pep356-related nucleotide sequences can be prepared by standard synthetic techniques, e.g., using an automated DNA synthesizer.

As used herein, the term “hybridizes to” is intended to describe conditions for moderate stringency or high stringency hybridization, preferably where the hybridization and washing conditions permit nucleotide sequences at least 60% homologous to each other to remain hybridized to each other. Preferably, the conditions are such that sequences at least about 70%, more preferably at least about 80%, even more preferably at least about 85%, 90%, 95% or 98% homologous to each other typically remain hybridized to each other. Stringent conditions are known to those skilled in the art and can be found in Current Protocols in Molecular Biology, John Wiley & Sons, N.Y. (1989), 6.3.1-6.3.6. A preferred, non-limiting example of stringent hybridization conditions are as follows: the hybridization step is realized at 65° C. in the presence of 6×SSC buffer, 5× Denhardt's solution, 0,5% SDS and 100 μg/ml of salmon sperm DNA. The hybridization step is followed by four washing steps:

two washings during 5 m, preferably at 65° C. in a 2×SSC and 0.1% SDS buffer;

one washing during 30 min, preferably at 65° C. in a 2×SSC and 0.1% SDS buffer,

one washing during 10 min, preferably at 65° C. in a 0.1×SSC and 0.1% SDS buffer,

these hybridization conditions being suitable for a nucleic acid molecule of about 20 nucleotides in length. It will be appreciated that the hybridization conditions described above are to be adapted according to the length of the desired nucleic acid, following techniques well known to the one skilled in the art, for example be adapted according to the teachings disclosed in Hames B. D. and Higgins S. J. (1985) Nucleic Acid Hybridization: A Practical Approach, Hames and Higgins Ed., IRL Press, Oxford; and Current Protocols in Molecular Biology.

“Percent homology” is used hereinto refer to both nucleic acid sequences and amino acid sequences. To determine the percent homology of two amino acid sequences or of two nucleic acids, the sequences are aligned for optimal comparison purposes (e.g., gaps can be introduced in the sequence of a first amino acid or nucleic acid sequence for optimal alignment with a second amino or nucleic acid sequence and non-homologous sequences can be disregarded for comparison purposes). In a preferred embodiment, the length of a reference sequence aligned for comparison purposes is at least 30%, preferably at least 40%, more preferably at least 50%, even more preferably at least 60%, and even more preferably at least 70%, 80%, 90% or 95% of the length of the reference sequence. The amino acid residues or nucleotides at corresponding amino acid positions or nucleotide positions are then compared. When a position in the first sequence is occupied by the same amino acid residue or nucleotide as the corresponding position in the second sequence, then the molecules are homologous at that position (i.e., as used herein amino acid or nucleic acid “identity” is equivalent to amino acid or nucleic acid “homology”). The percent homology between the two sequences is a function of the number of identical positions shared by the sequences (i.e., % homology=# of identical positions/total # of positions 100).

The comparison of sequences and determination of percent homology between two sequences can be accomplished using a mathematical algorithm. A preferred, non-limiting example of a mathematical algorithm utilized for the comparison of sequences is the algorithm of Karlin and Altschul (1990) Proc. Natl. Acad. Sci. USA 87:2264-68, modified as in Karlin and Altschul (1993) Proc. Natl. Acad. Sci. USA 90:5873-77. Such an algorithm is incorporated into the NBLAST and XBLAST programs (version 2.0) of Altschul, et al. (1990) J. Mol. Biol. 215:403-10. BLAST nucleotide searches can be performed with the NBLAST program, score 100, wordlength=12 to obtain nucleotide sequences homologous to Pep356-related nucleic acid molecules of the invention. BLAST protein searches can be performed with the XBLAST program, score=50, wordlength=3 to obtain amino acid sequences homologous to Pep356-related peptide molecules of the invention. To obtain gapped alignments for comparison purposes, Gapped BLAST can be utilized as described in Altschul et al., (1997) Nucleic Acids Research 25(17):3389-3402. When utilizing BLAST and Gapped BLAST programs, the default parameters of the respective programs (e.g., XBLAST and NBLAST) can be used. See http://wvw.ncbi.nlm.nih.gov. Another preferred, non-limiting example of a mathematical algorithm utilized for the comparison of sequences is the algorithm of Myers and Miller, CABIOS (1989). Such an algorithm is incorporated into the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used.

The term “polypeptide” refers to a polymer of amino acids without regard to the length of the polymer; thus, peptides, oligopeptides, and proteins are included within the definition of polypeptide. This term also does not specify or exclude post-translational modifications of polypeptides. For example, polypeptides include those with covalent attachment of glycosyl, acetyl, phosphate, amide, lipid, carboxyl, acyl or carbohydrate groups. Also included within the definition are polypeptides which contain one or more analogs of an amino acid (including, for example, non-naturally occurring amino acids, amino acids which only occur naturally in an unrelated biological system, modified amino acids from mammalian systems etc.), polypeptides with substituted linkages, as well as other modifications known in the art, both naturally occurring and non-naturally occurring.

The term “protein” as used herein may be used synonymously with the term “polypeptide” or may refer to, in addition, a complex of two or more polypeptides which may be linked by bonds other than peptide bonds, for example, such polypeptides making up the protein may be linked by disulfide bonds. The term “protein” may also comprehend a family of polypeptides having identical amino acid sequences but different post-translational modifications, particularly as may be added when such proteins are expressed in eukaryotic hosts.

An “isolated” or “purified” protein or biologically active portion thereof is substantially free of cellular material or other contaminating proteins from the cell or tissue source from which the Pep356-related peptide, or a biologically active fragment or homologue thereof is derived, or substantially free from chemical precursors or other chemicals when chemically synthesized. The language “substantially free of cellular material” includes preparations of a protein according to the invention (e.g. Pep356-related peptide, or a biologically active fragment or homologue thereof) in which the protein is separated from cellular components of the cells from which it is isolated or recombinantly produced. In one embodiment, the language “substantially free of cellular material” includes preparations of a protein according to the invention having less than about 30% (by dry weight) of protein other than the Pep356-related peptide (also referred to herein as a “contaminating protein”), more preferably less than about 20% of protein other than the protein according to the invention, still more preferably less than about 10% of protein other than the protein according to the invention, and most preferably less than about 5% of protein other than the protein according to the invention. When the protein according to the invention or biologically active portion thereof is recombinantly produced, it is also preferably substantially free of culture medium, i.e., culture medium represents less than about 20%, more preferably less than about 10%, and most preferably less than about 5% of the volume of the protein preparation.

The language “substantially free of chemical precursors or other chemicals” includes preparations of Pep356-related peptide, or a biologically active fragment or homologue thereof in which the protein is separated from chemical precursors or other chemicals which are involved in the synthesis of the protein. In one embodiment, the language “substantially free of chemical precursors or other chemicals” includes preparations of a Pep356-related peptide having less than about 30% (by dry weight) of chemical precursors or non-Pep356-related peptides chemicals, more preferably less than about 20% chemical precursors or non-Pep356-related peptides chemicals, still more preferably less than about 10% chemical precursors or non-Pep356-related peptides chemicals, and most preferably less than about 5% chemical precursors or non-Pep356-related peptides chemicals.

The term “recombinant polypeptide” is used herein to refer to polypeptides that have been artificially designed and which comprise at least two polypeptide sequences that are not found as contiguous polypeptide sequences in their initial natural environment, or to refer to polypeptides which have been expressed from a recombinant polynucleotide. The term “Pep356 peptide” or “Pep356-related peptide” refers to a peptide comprising the sequence described by those selected from the group consisting of SEQ ID NOs:3-5. Such peptide may be post-translationally modified (e.g., amidated, phosphorylated, acylated, or glycosylated). Pep356 peptide may also contain other structural or chemical modifications such as disulfide linkages or amino acid side chain interactions such as hydrogen and amide bonds that result in complex secondary and tertiary structures.

The terms “Pep356-related peptide” and “Pep356-related peptides” are used herein to embrace any and all of the peptides, polypeptides and proteins of the present invention. These include mutant polypeptides, such as deletion, addition, swap, or truncation mutants, fusion polypeptides comprising such polypeptides, and polypeptide fragments of at least three, but preferably 8, 10, 12, 15, or 21 contiguous amino acids of the sequences of SEQ ID NOs:3-5. The invention embodies polypeptides encoded by the nucleic acid sequences of the Pep356 gene or messenger RNA, as well as the Pep356-related peptides from humans, including isolated or purified Pep356-related peptides consisting of, consisting essentially of, or comprising the sequence of SEQ ID NOs:3-5. Preferred Pep356-related peptides retain at least one biological activity of Pep356.

The term “biological activity” as used herein refers to any function carried out by Pep356. These include but are not limited to: (1) antigenicity, or the ability to bind an anti-Pep356-related peptide specific antibody; (2) inmmunogenicity, or the ability to generate an anti-Pep356-related peptide specific antibody; (3) interacting with a Pep356-related target molecule, preferably a protein, in particular, a microfibril associated protease such as elastase; (4) circulating in human plasma; (5) mediating an angiogenesis, particularly during lung development and wound healing; and (6) undergoing dibasic cleavage.

Another aspect of the invention pertains to anti-Pep356-related antibodies. The term “antibody” as used herein refers to immunoglobulin molecules and immunologically active portions of immunoglobulin molecules, i.e., molecules that contain an antigen binding site which specifically binds (immunoreacts with) an antigen, such as a Pep356-related peptide, or a biologically active fragment or homologue thereof. Examples of immunologically active portions of immunoglobulin molecules include F(ab) and F(ab′)₂ fragments which can be generated by treating the antibody with an enzyme such as pepsin. The invention provides polyclonal and monoclonal antibodies that bind a Pep356-related peptide, or a biologically active fragment or homologue thereof. The term “monoclonal antibody” or “monoclonal antibody composition”, as used herein, refers to a population of antibody molecules that contain only one species of an antigen binding site capable of immunoreacting with a particular epitope of a Pep356-related peptide. A monoclonal antibody composition thus typically displays a single binding affinity for a particular Pep356-related peptide with which it immnunoreacts.

As used herein, a “label group” is any compound that, when attached to a polynucleotide or polypeptide (including antibodies), allows for detection or purification of said polynucleotide or polypeptide. Label groups may be detected or purified directly or indirectly by a secondary compound, including an antibody specific for said label group. Useful label groups include radioisotopes (e.g., ³²P, ³⁵S, ³H, ¹²⁵I), fluorescentcompounds (e.g., 5-bromodesoxyuridin, umbelliferone, fluorescein, fluorescein isothiocyanate, rhodamine, dichlorotriazinylamine fluorescein, dansyl chloride, phycoerythrin acetylaminofluorene, digoxigenin), luminescent compounds (e.g., luminol, GFP, luciferin, aequorin), enzymes or enzyme co-factor detectable labels (e.g., peroxidase, luciferase, alkaline phosphatase, galactosidase, or acetylcholinesterase), or compounds that are recognized by a secondary factor such as strepavidin, GST, or biotin. Preferably, a label group is attached to a polynucleotide or polypeptide in such a way as to not interfere with the biological activity of the polynucleotide or polypeptide.

Radioisotopes may be detected by direct counting of radioemission, film exposure, or by scintillation counting, for example. Enzymatic labels may be detected by determination of conversion of an appropriate substrate to product, usually causing a fluorescent reaction. Fluorescent and luminescent compounds and reactions may be detected by, e.g., radioemission, fluorescent microscopy, fluorescent activated cell sorting, or a luminometer.

As used herein, the term “vector” refers to a nucleic acid molecule capable of transporting another nucleic acid to which it has been linked. One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal marmmalian vectors) are integrated into the genome of a host cell upon introduction into the host cell, and thereby are replicated along with the host genome. Moreover, certain vectors are capable of directing the expression of genes to which they are operatively linked. Such vectors are referred to herein as “expression vectors”. In general, expression vectors of utility in recombinant DNA techniques are often in the form of plasmids. In the present specification, “plasmid” and “vector” can be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

As used herein, “effective amount” describes the amount of an agent, preferably a Pep356-related peptide of the invention, sufficient to have a desired effect. As an example, an effective amount of a Pep356-related peptide is the amount of the peptide or biologically active fragment thereof required to stimulate angiogenesis by 5% or 10%. The effective amount for a particular patient may vary depending on such factors as the diagnostic method of the symptom being measured, the state of the condition being treated, the overall health of the patient, method of administration, and the severity of side-effects.

Pep356-related Peptides of the Invention

The Pep356-related peptides of the invention are listed as SEQ ID NO: 3 through 5. These peptides were found to be secreted and are predicted to be active peptides which are released from their precursors by dibasic cleavage.

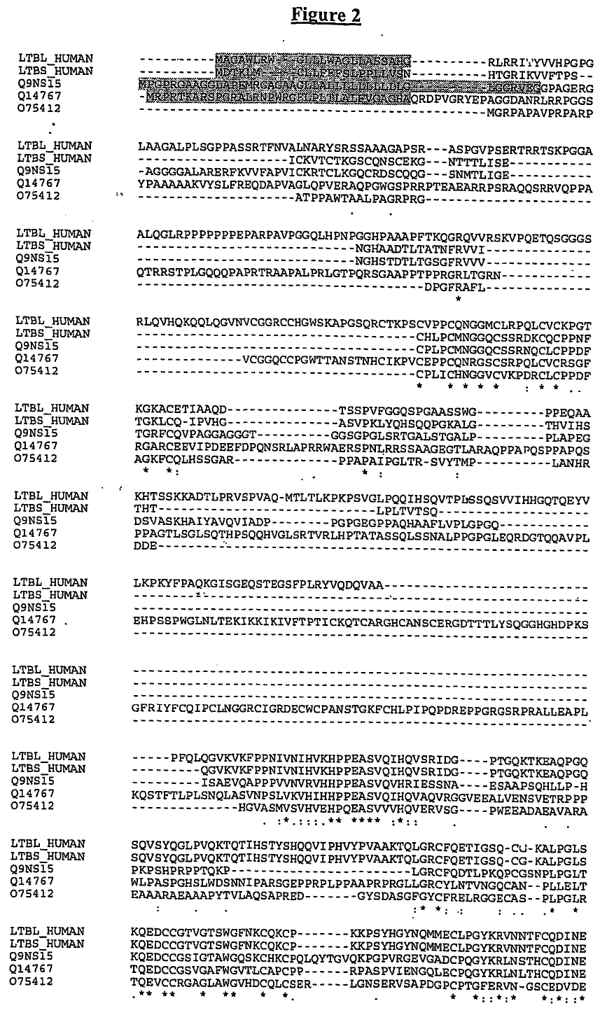

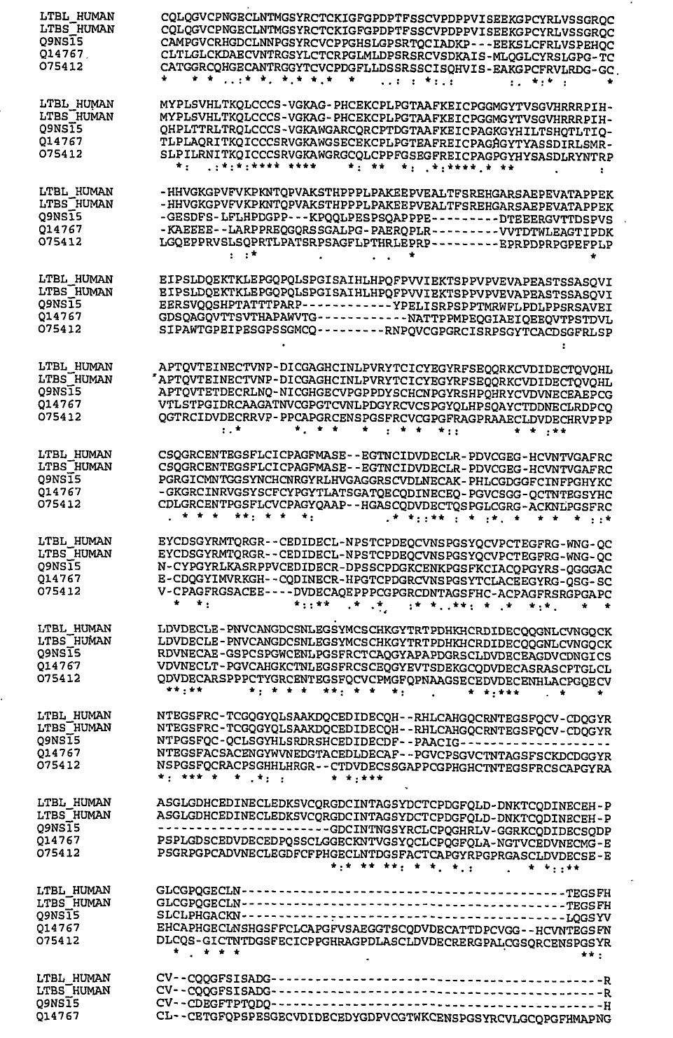

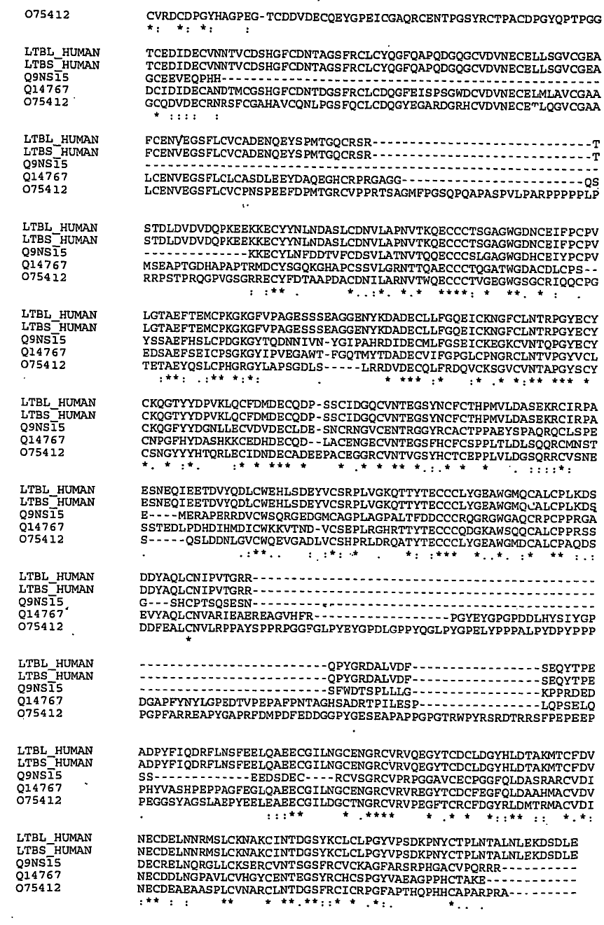

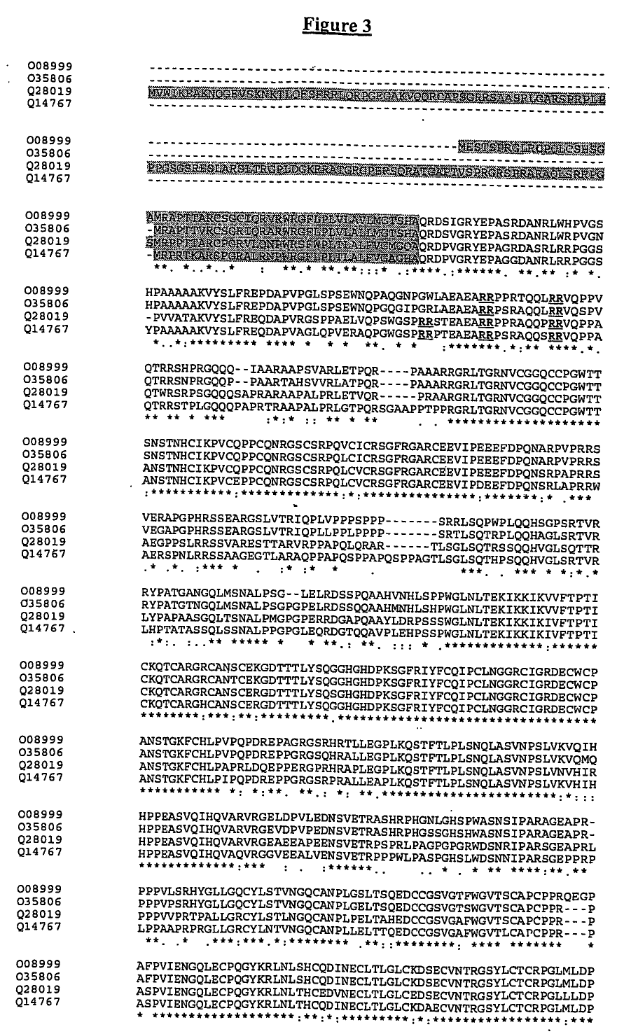

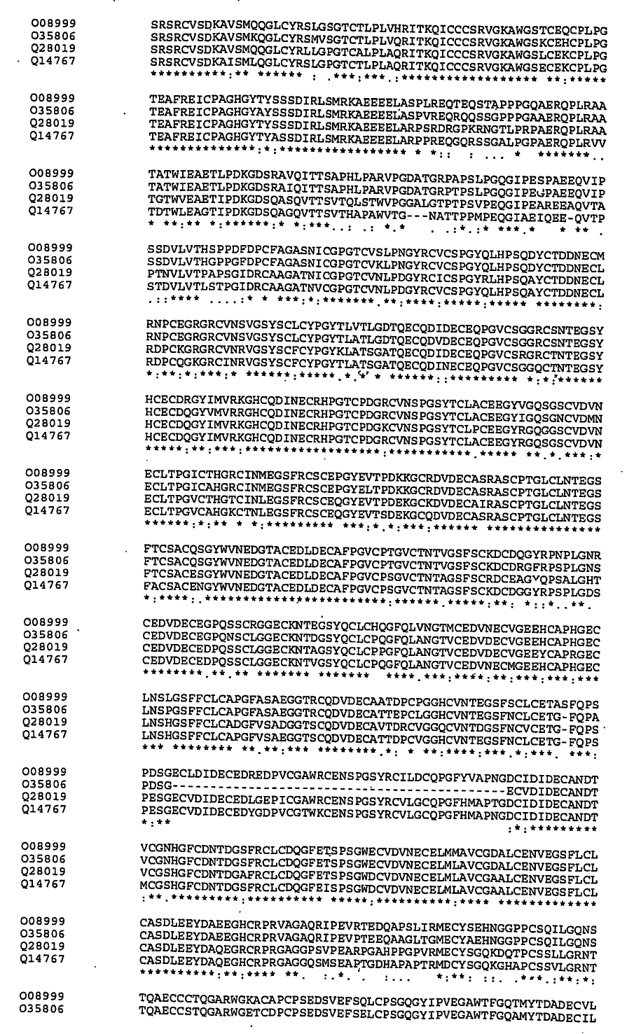

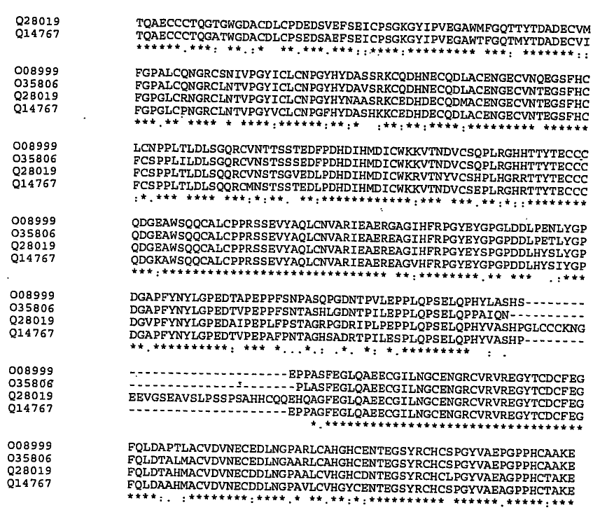

The Pep356-related peptides represent a sub-region of a latent transforming growvth factor binding protein (accession number Q14767, available at http://www.expasy.ch, and Moren A., et al., J. Biol. Chem. 269:32469-32478(1994)). Each of the peptides of SEQ ID NO: 3 to 5 correspond to the product of the cleavage at a different dibasic cleavage site of the mature form of the LTBP2 protein. The mature form of the LTBP2 protein is obtained from the precusor by cleavage of the signal peptide after position 35 (GHA-QR). SEQ ID NO: 1 and 2 represent the precursor and mature LTBP2 protein, respectively. Interestingly, LTBP2 diverges from other human LTBP family members mainly in its N-term, which gives rise to the secreted peptides of the invention (FIG. 2). However, as shown in FIG. 3, there is a very high interspecies conservation of the precusor protein sequence and of the Pep356-related peptides.

Moreover, the amino acid residue 1 from SEQ ID Nos:2-5, which is a glutarnine, is likely to undergo cyclisation to pyrrolidone carboxylic acid (Abraham G N and Podell D N, Mol Cell Biochem. 1981 Aug. 11; 38 Spec No(Pt 1):181-90). This may be critical for the biological activity of Pep356-related peptides (see, for example, Thyrotropin Releasing Hormone, Masson M A et al, Biochimie, 1979; 61(7):847-54).

The terms “Pep356-related peptide” and “Pep356-related peptides” are used herein to embrace any and all of the peptides, polypeptides and proteins of the present invention. Also forming part of the invention are polypeptides encoded by the polynucleotides of the invention, as well as fusion polypeptides comprising such polypeptides. The invention embodies polypeptides encoded by the nucleic acid sequences of the Pep356 gene, as well as the Pep356-related peptides from humans, including isolated or purified Pep356-related peptides consisting of, consisting essentially of, or comprising the sequence selected from the group consisting of SEQ ID NOs:3-5.

The present invention embodies isolated, purified, and recombinant polypeptides comprising a contiguous span of at least 3 amino acids, preferably at least 8 to 10 amino acids. In preferred embodiments the contiguous stretch of amino acids comprises the site of a mutation or functional mutation, including a deletion, addition, swap or truncation of the amino acids in the Pep356-related peptide sequence. The invention also concerns the polypeptide encoded by the Pep356-related nucleotide sequences of the invention, or a complementary sequence thereof or a fragment thereof.

One aspect of the invention pertains to isolated Pep356-related peptides, and biologically active portions thereof, as well as polypeptide fragments suitable for use as immunogens to raise anti-Pep356-related antibodies. In one embodiment, native Pep356-related peptides can be isolated from cells or tissue sources by an appropriate purification scheme using standard protein purification techniques. In another embodiment, Pep356-related peptides are produced by recombinant DNA techniques. Alternative to recombinant expression, a Pep356-related peptide or polypeptide can be synthesized chemically using standard peptide synthesis techniques.

Typically, biologically active portions comprise a domain or motif with at least one activity of a Pep356-related peptide. In a preferred embodiment, a Pep356-related peptide comprises a Pep356-related-target binding region, a dibasic cleavage site, or a signal sequence.

A biologically active Pep356-related peptide may, for example, comprise at least 1, 2, 3, or 5 amino acid changes from the sequence selected from the group consisting of SEQ ID NOs:3-5, or may encode a biologically active Pep356-related peptide comprising at least 1%, 2%, 3%, 5%, 8%, 10% or 15% changes in amino acids from the sequence selected from the group consisting of SEQ ID NOs:3-5.

In other embodiments, the Pep356-related peptide is substantially homologous to the sequence selected from the group consisting of SEQ ID NOs:3-5, and retains the functional activity of the Pep356-related peptide, yet differs in amino acid sequence due to natural allelic variation or mutagenesis, as described further herein. Accordingly, in another embodiment, the Pep356-related peptide is a protein which comprises an amino acid sequence which shares more than about 60% but iess than 100% homology with the amino acid sequence selected from the group consisting of SEQ ID NOs:3-5 and retains the functional activity of the Pep356-related peptides of SEQ ID NOs:3-5. Preferably, the protein is at least about 30%, 40%, 50%, 60%, 70%, 80%, 85%, 90%, 92%, 95%, 97%, 98%, 99% or 99.8% homologous to a sequence selected from the group consisting of SEQ ID NOs:3-5, but is not identical to SEQ ID NOs:3-5. Preferably the Pep356-related peptide is less than identical (e.g. 100% identity) to a naturally occurring Pep356-related peptides. Percent homology can be determined as further detailed above.

Pep356-related Nucleic Acids

One aspect of the invention pertains to purified or isolated nucleic acid molecules that encode Pep356-related peptides or biologically active portions thereof, as well as nucleic acid fragments thereof. Said nucleic acids may be used for example in therapeutic and diagnostic methods and in drug screening assays as further described herein.

An object of the invention is a purified, isolated, or recombinant nucleic acid coding for a Pep356-related peptide, complementary sequences thereto, and fragments thereof. The invention also pertains to a purified or isolated nucleic acid comprising a polynucleotide having at least 95% nucleotide identity with a polynucleotide coding for a Pep356-related peptide, advantageously 99% nucleotide identity, preferably 99.5% nucleotide identity and most preferably 99.8% nucleotide identity with a polynucleotide coding for a Pep356-related peptide, or a sequence complementary thereto or a biologically active fragment thereof. Another object of the invention relates to purified, isolated or recombinant nucleic acids comprising a polynucleotide that hybridizes, under the stringent hybridization conditions defined herein, with a polynucleotide coding for a Pep356-related peptide, or a sequence complementary thereto or a variant thereof or a biologically active fragment thereof.

In another preferred aspect, the invention pertains to purified or isolated nucleic acid molecules that encode a portion or variant of a Pep356-related peptide, wherein the portion or variant displays a Pep356-related activity of the invention. Preferably said portion or variant is of a naturally-occurring Pep356-related peptide.

Another object of the invention is a purified, isolated, or recombinant nucleic acid encoding a Pep356-related peptide comprising, consisting essentially of, or consisting of the amino acid sequence of SEQ ID NOS 3 to 5, or fragments thereof, wherein the isolated nucleic acid molecule encodes one or more Pep356-related motifs. For example, the purified, isolated or recombinant nucleic acid rpay comprise a genomic DNA or fragment thereof which encodes the polypeptide of SEQ ID NOS 3 to 5 or a fragment thereof. Any combination of said motifs may also be specified.

The nucleotide sequence determined from the cloning of the Pep356 gene allows for the generation of probes and primers designed for use in identifying and/or cloning other Pep356-related peptides family members (e.g. sharing the novel functional domains), as well as Pep356-related peptides homologues from other species.

A nucleic acid fragment encoding a “biologically active portion of a Pep356-related peptide” can be prepared by isolating a portion of a nucleotide sequence coding for a Pep356-related peptide, which encodes a polypeptide having a Pep356-related peptides biological activity, expressing the encoded portion of the Pep356-related peptide (e.g., by recombinant expression in vitro or in vivo) and assessing the activity of the encoded portion of the Pep356-related peptide.

The invention fu rther encompasses nucleic acid molecules that differ from the Pep356-related nucleotide sequences of the invention due to degeneracy of the genetic code and encode the same Pep356-related peptides and fragment of the invention.

In addition to the Pep356-related nucleotide sequences described above, it will be appreciated by those skilled in the art that DNA sequence polymorphisms that lead to changes in the amino acid sequences of the Pep356-related peptides may exist within a population (e.g., the human population). Such genetic polymorphism may exist among individuals within a population due to natural allelic variation. Such natural allelic variations can typically result in 1-5% variance in the nucleotide sequence of the Pep356 gene or nucleic acid sequence encoding Pep356-related peptide.

Nucleic acid molecules corresponding to natural allelic variants and homologues of the Pep356-related nucleic acids of the invention can be isolated based on their homology to the Pep356-related nucleic acids disclosed herein using the cDNAs disclosed herein, or a portion thereof, as a hybridization probe according to standard hybridization techniques under stringent hybridization conditions.

It will be appreciated that the invention comprises polypeptides having an amino acid sequence encoded by any of the polyinucleotides of the invention.

Uses of Pep356-related Nucleic Acids

Polynucleotide sequences (or the complements thereof) encoding Pep356-related peptides have various applications, including uses as hybridization probes, in chromosome and gene mapping, and in the generation of antisense RNA and DNA. In addition, Pep356-related peptides-encoding nucleic acids are usefwil as targets for pharmaceutical intervention, e.g. for the development of DNA vaccines, and for the preparation of Pep356-related peptides by recombinant techniques, as described herein. The polynucleotides described herein, including sequence variants thereof, can be used in diagnostic assays. Accordingly, diagnostic methods based on detecting the presence of such polynucleotides in body fluids or tissue samples are a feature of the present invention. Examples of nucleic acid based diagnostic assays in accordance with the present invention include, but are not limited to hybridization assays, e.g., in situ hybridization, and PCR-based assays. Polynucleotides, including extended length polynucleotides, sequence variants and fragments thereof, as described herein, may be used to generate hybridization probes or PCR primers for use in such assays. Such probes and primers will be capable of detecting polynucleotide sequences, including genomic sequences that are similar, or complementary to, the Pep356-related peptides polynucleotides described herein.

The invention includes primer pairs for carrying out a PCR to amplify a segment of a polynucleotide of the invention. Each primer of a pair is an oligonucleotide having a length of between 15 and 30 nucleotides such that i) one primer of the pair forms a perfectly matched duplex with one strand of a polynucleotide of the invention and the other primer of the pair form a perfectly match duplex with the complementary strand of the same polynucleotide, and ii) the primers of a pair form such perfectly matched duplexes at sites on the polynucleotide that separated by a distance of between 10 and 2500 nucleotides. Preferably, the annealing temperature of each primer of a pair to its respective complementary sequence is substantially the same.

Hybridization probes derived from polynucleotides of the invention can be used, for example, in performing in situ hybridization on tissue samples, such as fixed or frozen tissue sections prepared on microscopic slides or suspended cells. Briefly, a labeled DNA or RNA probe is allowed to bind its DNA or RNA target sample in the tissue section on a prepared microscopic, under controlled conditions. Generally, dsDNA probes consisting of the DNA of interest cloned into a plasmid or bacteriophage DNA vector are used for this purpose, although ssDNA or ssRNA probes may also be used. Probes are generally oligonucleotides between about 15 and 40 nucleotides in length. Alternatively, the probes can be polynucleotide probes generated by PCR random priming primer extension or in vitro transcription of RNA from plasmids (riboprobes). These latter probes are typically several hundred base pairs in length. The probes can be labeled by any of a number of label groups and the particular detection method will correspond to the type of label utilized on the probe (e.g., autoradiography, X-ray detection, fluorescent or visual microscopic analysis, as appropriate). The reaction can be further amplified in situ using immunocytochemical techniques directed against the label of the detector molecule used, such antibodies directed to a fluorescein moiety present on a fluorescently labeled probe, or against avidin, or marker enzymes (peroxidase, alkaline phosphatase). Specific labeling and in situ detection methods can be found, for example, in Howard, G. C., Ed., Methods in Nonradioactive Detection, Appleton & Lange, Norwalk, Conn., (1993).

Hybridization probes and PCR primers may also be selected from the genomic sequences corresponding to the full-length proteins identified in accordance with the present invention, including promoter, enhancer elements and introns of the gene encoding the naturally occurring polypeptide. Nucleotide sequences encoding a Pep356-related peptide can also be used to construct hybridization probes for mapping the gene encoding Pep356-related peptides and for the genetic analysis of individuals. The.nucleotide sequences provided herein may be mapped to a chromosome and specific regions of a chromosome using known techniques, such as in situ hybridization, linkage analysis against known chromosomal markers, and hybridization screening with libraries. Briefly, sequences can be mapped to chromosomes by preparing PCR primers (preferably 15-25 bp) from the Pep356-related peptides cDNA. Computer analysis of the 3′ untranslated region is used to rapidly select primers that do not span more than one exon in the genomic DNA, which would complicate the amplification process. Individuals carrying variations of, or mutations in the gene encoding a Pep356-related peptide of the present invention may be detected at the DNA level by a variety of techniques. Nucleic acids used for diagnosis may be obtained from a patient's cells, including, for example, tissue biopsy and autopsy material. Genomic DNA may be used directly for detection or may be amplified enzymatically by using PCR (Saiki, et al. Nature 324:163-166 (1986)) prior to analysis. RNA or CDNA may also be used for the same purpose. As an example, PCR primers complementary to the nucleic acid of the present invention can be used to identify and analyze mutations in the gene of the present invention. Deletions and insertions can be detected by a change in size of the amplified product in comparison to the normal genotype. Point mutations can be identified by hybridizing asmplified DNA to radiolabeled RNA of the invention or altematively, radiolabeled antisense DNA sequences of the invention. Sequence changes at specific locations may also be revealed by nuclease protection assays, such RNase and S1 protection or the chemical cleavage method (Cotton, et al., Proc. Natl. Acad. Sci. USA 85:4397-4401 (1985)), or by differences in melting temperatures. “Molecular beacons” (Kostrikis L. G. et al., Science 279:1228-1229 (1998)), hairpin-shaped, single-stranded synthetic oligonucleotides containing probe sequences which are complementary to the nucleic acid of the present invention, may also be used to detect point mutations or other sequence changes as well as monitor expression levels of Pep356-related peptides.

Oligonucleotide and Antisense Compounds

Oligonucleotides of the invention, including PCR primers and antisense compounds, are synthesized by conventional means on a commercially available automated DNA synthesizer, e.g. an Applied Biosystems (Foster City, Calif.) model 380B, 392 or 394 DNA/RNA synthesizer, or like instrument. Preferably, phosphoramidite chemistry is employed, e.g. as disclosed in the following references: Beaucage and Iyer, Tetrahedron, 48: 2223-2311 (19.92); Molko et al, U.S. Pat. No. 4,980,460; Koster et al, U.S. Pat. No. 4,725,677; Caruthers et al, U.S. Pat. Nos. 4,415,732; 4,458,066; and 4,973,679. For therapeutic use, nuclease resistant backbones are preferred. Many types of modified oligonucleotides are available that confer nuclease resistance, e.g. phosphorothioate, phosphorodithioate, phosphoramidate. For references see, e.g. phosphorothioates: Stec et al, U.S. Pat. No. 5,151,510; Hirschbein, U.S. Pat. No. 5,166,387; Bergot, U.S. Pat. No. 5,183,885; phosphoramidates: Froehler et al, International application PCT/US90/03138; and for a review of additional applicable chemistries: Uhlmann and Peyman (cited above). The length of the antisense oligonucleotides has to be sufficiently large to ensure that specific binding will take place only at the desired target polynucleotide and not at other fortuitous sites. The upper range of the length is determined by several factors, including the inconvenience and expense of synthesizing and purifying oligomers greater than about 30-40 nucleotides in length, the greater tolerance of longer oligonucleotides for mismatches than shorter oligonucleotides, and the like. Preferably, the antisense oligonucleotides of the invention have lengths in the range of about 15 to 40 nucleotides. More preferably, the oligonucleotide moieties have lengths in the range of about 18 to 25 nucleotides.

Primers and Probes

Primers and probes of the invention can be prepared by any suitable method, including, for example, cloning and restriction of appropriate sequences and direct chemical synthesis by a method such as the phosphodiester method of Narang S A et al (Methods Enzymol 1979;68:90-98), the phosphodiester method of Brown E L et al (Methods Enzymol 1979;68:109-15 1), the diethylphosphoramidite method of Beaucage et al (Tetrahedron Lett 1981, 22: 1859-1862) and the solid support method described in EP 0 707 592.

Detection probes are generally nucleic acid sequences or uncharged nucleic acid analogs such as, for example peptide nucleic acids which are disclosed in WO 92/20702, morpholino analogs which are described in U.S. Pat. Nos. 5,185,444; 5,034,506 and 5,142,047. If desired, the probe may be rendered “non-extendable” in that additional dNTPs cannot be added to the probe. In and of themselves analogs usually are non-extendable and nucleic acid probes can be rendered non-extendable by modifying the 3′ end of the probe such that the hydroxyl group is no longer capable of participating in elongation. For example, the 3′ end of the probe can be functionalized with the capture or detection label to thereby consume or otherwise block the hydroxyl group.

Any of the polynucleotides of the present invention can be labeled, if desired, by incorporating any label group known in the art to be detectable by spectroscopic, photochemical, biochemical, imnmunochemical, or chemical means. Additional examples include non-radioactive labeling of nucleic acid fragments as described in Urdea et al. (Nucleic Acids Research. 11:4937-4957, 1988) or Sanchez-Pescador et al. (J. Clin. Microbiol. 26(10): 1934-1938, 1988). In addition, the probes according to the present invention may have structural characteristics such that they allow the signal amplification, such structural characteristics being, for example, branched DNA probes as those described by Urdea et al (Nucleic Acids Symp. Ser. 24:197-200, 1991) or in the European patent No. EP 0225807 (Chiron).

A label can also be used to capture the primer, so as to facilitate the immobilization of either the primer or a primer extension product, such as amplified DNA, on a solid support. A capture label is attached to the primers or probes and can be a specific binding member which fortns a binding pair with the solid's phase reagent's specific binding member (e.g. biotin and streptavidin). Therefore depending upon the type of label carried by a polynucleotide or a probe, it may be employed to capture or to detect the target DNA. Further, it will be understood that the polynucleotides, primers or probes provided herein, may, themselves, serve as the capture label. For example, in the case where a solid phase reagent's binding member is a nucleic acid sequence, it may be selected such that it binds a complementary portion of a primer or probe to thereby immobilize the primer or probe to the solid phase. In cases where a polynucleotide probe itself serves as the binding member, those skilled in the art will recognize that the probe will contain a sequence or “tail” that is not complementary to the target. In the case where a polynucleotide primer itself serves as the capture label, at least a portion of the primer will be free to hybridize with a nucleic acid on a solid phase. DNA labeling techniques are well known to the skilled technician. The probes of the present invention are useful for a number of purposes. They can be notably used in Southern hybridization to genomic DNA. The probes can also be used to detect PCR amplification products. They may also be used to detect mismatches in Pep356-encoding genes or mRNA using other techniques.

Any of the nucleic acids, polynucleotides, primers and probes of the present invention can be conveniently immobilized on a solid support. Solid supports are known to those skilled in the art and include the walls of wells of a reaction tray, test tubes, polystyrene beads, magnetic beads, nitrocellulose strips, membranes, microparticles such as latex particles, sheep (or other animal) red blood cells, duracytes and others. The solid support is not critical and can be selected by one skilled in the art. Thus, latex particles, microparticles, magnetic or nonmagnetic beads, membranes, plastic tubes, walls of microtiter wells, glass or silicon chips, sheep (or other suitable animal's) red blood cells and duracytes are all suitable examples. Suitable methods for immobilizing nucleic acids on solid phases include ionic, hydrophobic, covalent interactions and the like. A solid support, as used herein, refers to any material which is insoluble, or can be made insoluble by a subsequent reaction. The solid support can be chosen for its intrinsic ability to attract and immobilize the capture reagent. Alternatively, the solid phase can retain an additional receptor which has the ability to attract and immobilize the capture reagent. The additional receptor can include a charged substance that is oppositely charged with respect to the capture reagent itself or to a charged substance conjugated to the capture reagent. As yet another alternative, the receptor molecule can be any specific binding member attached to the solid support and which has the ability to immobilize the capture reagent through a specific binding reaction. The receptor molecule enables the indirect binding of the capture reagent to a solid support material before the performance of the assay or during the performance of the assay. The solid phase thus can be a plastic, derivatized plastic, magnetic or non-magnetic metal, glass or silicon surface of a test tube, microtiter well, sheet, bead, microparticle, chip, sheep (or other suitable animal's) red blood cells, duracytes and other configurations known to those of ordinary skill in the art. The nucleic acids, polynucleotides, primers and probes of the invention can be attached to or immobilized on a solid support individually or in groups of at least 2, 5, 8, 10, 12, 15, 20, or 25 distinct polynucleotides of the invention to a single solid support. In addition, polynucleotides other than those of the invention may be attached to the same solid support as one or more polynucleotides of the invention.

Any polynucleotide provided herein may be attached in overlapping areas or at random locations on a solid support. Alternatively the polynucleotides of the invention may be attached in an ordered array wherein each polynucleotide is attached to a distinct region of the solid support which does not overlap with the attachment site of any other polynucleotide. Preferably, such an ordered array of polynucleotides is designed to be “addressable” where the distinct locations are recorded and can be accessed as part of an assay procedure. Addressable polynucleotide arrays typically comprise a plurality of different oligonucleotide probes that are coupled to a surface of a substrate in different known locations. The knowledge of the precise location of each polynucleotides location makes these “addressable” arrays particularly useful in hybridization assays. Any addressable array teclnology known in the art can be employed with the polynucleotides of the invention. One particular embodiment of these polynucleotide arrays is known as the Genechips, and has been generally described in U.S. Pat. No. 5,143,854; PCT publications WO 90/15070 and 92/10092.

Methods for Obtaining Variant Nucleic Acids and Polypeptides

In addition to naturally-occurring allelic variants of the Pep356-related peptides sequences that may exist in the population, the skilled artisan will appreciate that changes can be introduced by mutation into the nucleotide sequences coding for Pep356-related peptides, thereby leading to changes in the amino acid sequence of the encodead Pep356-related peptides, with or without altering the functional ability of the Pep356-related peptides.

Several types of variants are contemplated including 1) one in which one or more of the amino acid residues are substituted with a conserved or non-conserved amino acid residue and such substituted amino acid residue may or may not be one encoded by the genetic code, or 2) one in which one or more of the amino acid residues includes a substituent group, or 3) one in which the mutated Pep356-related peptide is fused with another compound, such as a compound to increase the half-life of the polypeptide (for example, polyethylene glycol), or 4) one in which the additional amino acids are fused to the mutated Pep356-related peptide, such as a leader or signal-anchor sequence or a sequence which is employed for purification of the mutated Pep356-related peptide or a preprotein sequence. Such variants are deemed to be within the scope of those skilled in the art.

For example, nucleotide substitutions leading to amino acid substitutions can be made in the sequences that do not substantially change the biological activity of the protein. An amino acid residue-can be altered from the wild-type sequence encoding a Pep356-related peptide, or a biologically active fragment or homologue thereof without altering the biological activity. In general, amino acid residues that are conserved among the Pep356-related peptides of the present invention, are predicted to be less amenable to alteration.

In one aspect, the invention pertains to nucleic acid molecules encoding Pep356-related peptides, or biologically active fragments or homologues thereof that contain changes in amino acid residues that are not essential for activity. Such Pep356-related peptides differ in amino acid sequence from SEQ ID NOS 3 to 5 yet retain biological activity. In one embodiment, the isolated nucleic acid molecule comprises a nucleotide sequence encoding a protein, wherein the protein comprises an amino acid sequence at least about 60% homologous to an amino acid sequence selected from the group consisting of SEQ ID NOS 3 to 5. Preferably, the protein encoded by the nucleic acid molecule is at least about 65-70% homologous to an amino acid sequence selected from the group consisting of SEQ ID NOS 3 to 5, more preferably sharing at least about 75-80% identity with an amino acid sequence selected from the group consisting of SEQ ID NOS 3 to 5, even more preferably sharing at least about 85%, 90%, 92%, 95%, 97%, 98%, 99% or 99.8% identity with an amino acid sequence selected from the group consisting of SEQ ID NOS 3 to 5.

In another aspect, the invention pertains to nucleic acid molecules encoding Pep356-related peptides that contain changes in amino acid residues that result in increased biological activity, or a modified biological activity. In another aspect, the invention pertains to nucleic acid molecules encoding Pep356-related peptides that contain changes in amino acid residues that are essential for a Pep356-related activity. Such Pep356-related peptides differ in amino acid sequence from SEQ ID NOS 3 to 5 and display reduced activity, or essentially lack one or more Pep356-related peptides biological activities.

Mutations can be introduced into any of SEQ ID NOS 3 to 5, by standard techniques, such as site-directed mutagenesis and PCR-mediated mutagenesis. For example, conservative amino acid substitutions may be made at one or more predicted non-essential amino acid residues. A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, a predicted nonessential amino acid residue in a Pep356-related peptide, or a biologically active fragment or homologue thereof may be replaced with another amino acid residue from the same side chain family. Alternatively, in another embodiment, mutations can be introduced randomly along all or part of a Pep356-related peptide coding sequence, such as by saturation mutagenesis, and the resultant mutants can be screened for Pep356-related peptide biological activity to identify mutants that retain activity. Following mutagenesis of one of SEQ ID NOS 3 to 5, the encoded protein can be expressed recombinantly and the activity of the protein can be determined.

The invention also provides Pep356-related chimeric or fusion proteins. As used herein, a Pep356-related “chimeric protein” or “fusion protein” comprises a Pep356-related peptide of the invention or fragment thereof, operatively linked, preferably fused in frame, to a non-Pep356-related peptide. In a preferred embodiment, a Pep356-related fusion protein comprises at least one biologically active portion of a Pep356-related peptide. In another preferred embodiment, a Pep356-related fusion protein comprises at least two biologically active portions of a Pep356-related peptide. For example, in one embodiment, the fusion protein is a GST-Pep356-related fusion protein in which a Pep356-related domain sequences are fused to the C-terminus of the GST sequences. Such fusion proteins can facilitate the purification of recombinant Pep356-related peptides. In another embodiment, the fusion protein is a Pep356-related peptide containing a heterologous signal sequence at its N-terminus, such as for example to allow for a desired cellular localization in a certain host cell.

The Pep356-related fusion proteins of the invention can be incorporated into pharmaceutical compositions and adrninistered to a subject in vivo. Moreover, the Pep356-related fusion proteins of the invention can be used as inmmunogens to produce anti-Pep356-related antibodies in a subject, to purify Pep356-related peptides ligands and in screening assays to identify molecules which inhibit the interaction of Pep356-related peptides polpeptide with a Pep356-related target molecule.

Furthermore, isolated peptidyl portions of the subject Pep356-related peptides can also be obtained by screening peptides recombinantly produced from the corresponding fragment of the nucleic acid encoding such peptides. In addition, fragments can be chemically synthesized using techniques known in the art such as conventional Merrifield solid phase f-Moc or t-Boc chemistry. For example, a Pep356-related peptide of the present invention may be arbitrarily divided into fragments of desired length with no overlap of the fragments, or preferably divided into overlapping fragments of a desired length. The fragments can be produced (recombinantly or by chemical synthesis) and tested to identify those peptidyl fragments which can function as either agonists or antagonists of a Pep356-related activity (e.g., by microinjection or in vitro protein binding assays). In an illustrative embodiment, peptidyl portions of a Pep356-related peptide, such as a Pep356-related target binding region, can be tested for Pep356-related activity by expression as thioredoxin fusion proteins, each of which contains a discrete fragment of the Pep356-related peptide (U.S. Pat. Nos. 5, 270,181 and 5,292,646; and W094/02502).

The present invention also pertains to variants of Pep356-related peptides which function as either Pep356-related antagonists or agonists. Variants of the Pep356-related peptides can be generated by mutagenesis, e.g., discrete point mutation or truncation of a Pep356-related peptide. An agonist of a Pep356-related peptide can retain substantially the same, or a subset, of the biological activities of the naturally occurring form of a Pep356-related peptide. An antagonist of a Pep356-related peptide can inhibit one or mnore of the activities of the naturally occurring form of the Pep356-related peptide by, for example, competitively inhibiting the association of Pep356-related peptide with a Pep356-related target molecule. Thus, specific biological effects can be elicited by treatment with a variant of limited function. In one embodiment, variants of a Pep356-related peptide which function as either Pep356-related peptides agonists (mimetics) or as Pep356-related peptides antagonists can be identified by screening combinatorial libraries of mutants, e.g., truncation mutants, of a Pep356-related peptide for Pep356-related peptide agonist or antagonist activity. In one embodiment, a variegated library of Pep356-related peptides variants is generated by combinatorial mutagenesis at the nucleic acid level and is encoded by a variegated gene library. A variegated library of Pep356-related peptides variants can be produced by, for example, enzymatically ligating a mixture of synthetic oligonucleotides into gene sequences such that a degenerate set of potential Pep356-related peptides sequences is expressible as individual polypeptides, or alternatively, as a set of larger fusion proteins (e.g., for phage display) containing the set of Pep356-related peptides sequences therein. There are a variety of methods which can be used to produce libraries of potential Pep356-related peptides variants from a degenerate oligonucleotide sequence. Chemical synthesis of a degenerate gene sequence can be performed in an automatic DNA synthesizer, and the synthetic gene then ligated into an appropriate expression vector. Use of a degenerate set of genes allows for the provision, in one mixture, of all of the sequences encoding the desired set of potential Pep356-related peptides sequences.

In addition, libraries of fragments of a Pep356-related peptide coding sequence can be used to generate a variegated population of Pep356-related peptides fragments for screening and subsequent selection of variants of a Pep356-related peptide. In one embodiment, a library of coding sequence fragments can be generated by treating a double stranded PCR fragment of Pep356-related peptides coding sequence with a nuclease under conditions wherein nicking occurs only about once per molecule, denaturing the double stranded DNA, renaturing the DNA to form double stranded DNA which can include sense/antisense pairs from different nicked products, removing single stranded portions from reformed duplexes by treatment with S1 nuclease, and ligating the resulting fragment library into an expression vector. By this method, an expression library can be derived which encodes N-terminal, C-terminal and internal fragments of various sizes of the Pep356-related peptide.

Modified Pep356-related peptides can be used for such purposes as enhancing therapeutic or prophylactic efficacy, or stability (e.g., ex vivo shelf life and resistance to proteolytic degradation in vivo). Such modified peptides, when designed to retain at least one activity of the naturally occurring form of the protein, are considered functional equivalents of the Pep356-related peptide described in more detail herein. Such modified peptide can be produced, for instance, by amino acid substitution, deletion, or addition.

Whether a change in the amino acid sequence of a peptide results in a functional Pep356-related peptides homologue can be readily determined by assessing a Pep356-related biological activity of the variant peptide. Peptides in which more than one replacement has taken place can readily be tested in the same manner.

This invention further contemplates a method of generating sets of combinatorial mutants of the presently disclosed Pep356-related peptides, as well as truncation and fragmentation mutants, and is especially useful for identifying potential variant sequences which differ from a wild-type form by, for example, efficacy, potency and/or intracellular half-life. One purpose for screening such combinatorial libraries is to isolate novel Pep356-related peptide homologues which modulate the biological activities of the wild-type proteir, or alternatively, possess novel activities all together. For example, mutagenesis can give rise to Pep356-related homologs which have intracellular half-lives dramatically different than the corresponding wild-type protein. The altered protein can be rendered either more stable or less stable to proteolytic degradation, or cellular processes which result in destruction or inactivation of, a Pep356-related peptide. Such Pep356-related peptide homologues, and the genes which encode them, can be utilized to alter the envelope of expression for a particular recombinant Pep356-related peptide by modulating the half-life of the recombinant protein. For instance, a short half-life can give rise to more transient biological effects associated with a particular recombinant Pep356-related peptide and, when part of an inducible expression system, can allow tighter control of recombinant protein levels within a cell. As above, such proteins, and particularly their recombinant nucleic acid constructs, can be used in gene therapy protocols.

In an illustrative embodiment of this method, the amino acid sequences for a population of Pep356-related peptides homologs or other related proteins are aligned, preferably to promote the highest homology possible. Such a population of variants can include, for example, Pep356-related peptides homologs from one or more species, or Pep356-related peptides homologs from the same species but which differ due to mutation. Amino acids at each position of the aligned sequences are selected to create a degenerate set of combinatorial sequences. There are many ways by which the library of potential Pep356-related peptides homologs can be generated from a degenerate oligonucleotide sequence. Chemical synthesis of a degenerate gene sequence can be carried out in an automatic DNA synthesizer, and the synthetic genes then be ligated into an appropriate gene for expression. The purpose of a degenerate set of genes is to provide, in one mixture, all of the sequences encoding the desired set of potential Pep356-related peptides sequences. The synthesis of degenerate oligonucleotides is well known in the art (Narang, S A (1983) Tetrahedron 393; Itakura et al. (1981) Recombinant DNA, Proc 3rd Cleveland Sympos. Macromolecules, ed. A G Walton, Amsterdam: Elsevier pp. 273-289; Itakura et al. (1984) Annu. Rev. Biochem. 53:323; Itakura et al. (1984) Science 198:1056; Ike et al. (1983) Nucleic Acid Res. 11:477. Such techniques have been employed in the directed evolution of other proteins (Scott et al. (1990) Science 249:386-390; Roberts et al. (1992) PNAS 89:2429-2433; Devlin et al. (1990) Science 249: 404406; Cwirla et al. (1990) PNAS 87: 6378-6382; as well as U.S. Pat. Nos. 5,223,409, 5,198,346, and 5,096,815). Alternatively, other forms of mutagenesis can be utilized to generate a combinatorial library, particularly where no other naturally occurring homologs have yet been sequenced. For example, Pep356-related peptides homologs (both agonist and antagonist forms) can be generated and isolated from a library by screening using, for example, alanine scanning mutagenesis (Ruf et al. (1994) Biochemistry 33:1565-1572; Wang et al. (1994) J Biol. Chem. 269:3095-3099; Balint et al. (1993) Gene 137:109-118; Grodberg et al. (1993) Eur. J Biochem. 218:597-601; Nagashima et al. (1993) J Biol. Chem. 268:2888-2892; Lowman et al. (1991) Biochemistry 30:10832-10838; and Cunningham et al. (1989) Science 244:1081-1085), by linlker scanning mutagenesis (Gustin et al. (1.993) Virology 193:653-660; Brown et al. (1992) Mol. Cell Biol. 12:2644 2652; McKnight et al. (1982) Science 232:316); by saturation mutagenesis (Meyers et al. (1986) Science 232:613); by PCR mutagenesis (Leung et al. (1989) Method Cell Mol Biol 1: 1-19); or by random mutagenesis (Miller et al. (1992) A Short Course in Bacterial Genetics, CSHL Press, Cold Spring Harbor, N.Y.; and Greener et al. (1994) Strategies in Mol Biol 7:32-34).

A wide range of techniques are known in the art for screening gene products of combinatorial libraries made by point mutations, as well as for screening cDNA libraries for gene products having a certain property. Such techniques will be generally adaptable for rapid screening of the gene libraries generated by the combinatorial mutagenesis of Pep356-related peptides. The most widely used techniques for screening large gene libraries typically comprises cloning the gene library into replicable expression vectors, transforming appropriate cells with the resulting library of vectors, and expressing the combinatorial genes under conditions in which detection of a desired activity facilitates relatively easy isolation of the vector encoding the gene whose product was detected.

Each of the illustrative assays described below is amenable to high throughput analysis as necessary to screen large numbers of degenerate Pep356-related peptides sequences created by combinatorial mutagenesis techniques. In one screening assay, the candidate gene products are displayed on the surface of a cell or viral particle, and the ability of particular cells or viral particles to bind a Pep356-relateiLtarget molecule (for example a modified peptide substrate) via this gene product is detected in a “panning assay”. For instance, the gene library can be cloned into the gene for a surface membrane protein of a bacterial cell, and the resulting fusion protein detected by panning (Ladner et al., WO 88/06630; Fuchs et al. (1991) BioTechnology 9:1370-1371, and Goward et al. (1992) TIBS 18:136 140). In a similar fashion, fluorescently labeled Pep356-related target can be used to score for potentially functional Pep356-related peptides homologs. Cells can be visually inspected and separated under a fluorescence microscope, or, where the morphology of the cell permits, separated by a fluorescence-activated cell sorter.

In an alternate embodiment, the gene library is expressed as a fusion protein on the surface of a viral particle. For instance, in the filamentous phage system, foreign peptide sequences can be expressed on the surface of infectious phage, thereby conferring two significant benefits. As phage can be applied to affinity matrices at very high concentrations, a large number of phage can be screened at one time. Second, since each infectious phage displays the combinatorial gene product on its surface, if a particular phage is recovered from an affinity matrix in low yield, the phage can be amplified by another round of infection. The group of almost identical E. coli filamentous phages M13, fd, and fl are most often used in phage display libraries, as either of the phage gIII or gVIII coat proteins can be used to generate fusion proteins without disrupting the ultimate packaging of the viral particle (Ladner et al. PCT publication WO 90/02909; Garrard et al., PCT publication WO 92/09690; Marks et al. (1992) J Biol. Chem. 267:16007-16010; Griffiths et al. (1993) EMBO J 12:725-734; Clackson et al. (1991) Nature 352:624-628; and Barbas et al. (1992) PNAS 89:4457 4461). In an illustrative embodiment, the recombinant phage antibody system (RPAS, Pharmacia Catalog number 27-9400-01) can be easily modified for use. in expressing Pep356-related peptides combinatorial libraries, and the Pep356-related peptides phage library can be panned on inmmobilized Pep356-related target molecule (glutathione immobilized Pep356-related peptides target-GST fusion proteins or immobilized DNA). Successive rounds of phage amplification and panning can greatly enrich for Pep356-related peptides homologs which retain an ability to bind a Pep356-related target and which can subsequently be screened further for biological activities in automated assays, in order to distinguish between agonists and antagonists.

The invention also provides for identification and reduction to functional minimal size of the Pep356-related peptide functional domains to generate mimetics. Thus, such mutagenic techniques as described above are also useful to map the determinants of Pep356-related peptides which participate in protein-protein interactions. To illustrate, the critical residues of a Pep356-related peptide which. are involved in molecular recognition of the Pep356-related target can be determined and used to generate Pep356-related peptides target-13P-derived peptidomimetics that competitively inhibit binding of the Pep356-related peptide to the Pep356-related peptides target. By employing, for example, scanning mutagenesis to map the amino acid residues of a particular Pep356-related peptide involved in binding a Pep356-related peptides target, peptidomimetic compounds can be generated which mimic those residues in binding to a Pep356-related peptides target, and which, by inhibiting binding of the Pep356-related peptide to the Pep356-related target molecule, can interfere with the function of a Pep356-related peptide in transcriptional regulation of one or more genes. For instance, non hydrolyzable peptide analogs of such residues can be generated using retro-inverse peptides (e.g., U.S. Pat. Nos. 5,116,947 and 5,219,089; and Pallai et al. (1983) Int J Pept Protein Res 21:84-92), benzodiazepine (e.g., Freidinger et al. in Peptides: Chemistry and Biology, G. R. Marshall ed., ESCOM Publisher: Leiden, Netherlands, 1988), azepine (e.g., Huffinan et al., Peptides—Chemistry and Biology, G. R. Marshall ed., ESCOM Publisher: Leiden, Netherlands, 1988), substituted gamma lactam rings (Garvey et al. in Peptides: Chemistry and Biology, G. R. Marshall ed., ESCOM Publisher: Leiden, Netherlands, 1988), keto-methylene pseudopeptides (Ewenson et al. (1986) J Med Chem 29:295; and Ewenson et al. in Peptides: Structure and Function (Proceedings of the 9th American Peptide Symposium) Pierce Chemical Co. Rockland, Ill., 1985), P-turn dipeptide cores (Nagai et al. (1985) Tetrahedron Left 26:647; and Sato et al. (1986) J Chem Soc Perkin Trans 1: 123 1), and P-aminoalcohols (Gordon et al. (1985) Biochem Biophys Res Commun 126:419; and Dann et al. (1986) Biochem Biophys Res Commun 134:71).

Chemical Manufacture of Pep356-related Compositions

Peptides of the invention are synthesized by standard techniques, e.g. Stewart and Young, Solid Phase Peptide Synthesis, 2nd Ed. (Pierce Chemical Company, Rockford, Ill., 1984). Preferably, a commercial peptide synthesizer is used, e.g. Applied Biosystems, Inc. (Foster City, Calif.) model 430A, and polypeptides of the invention may be assembled from multiple, separately synthesized and purified, peptide in a convergent synthesis approach, e.g. Kent et al, U.S. Pat. No. 6,184,344 and Dawson and Kent, Annu. Rev. Biochem., 69: 923-960 (2000). Peptides of the invention may be assembled by solid phase synthesis on a cross-linked polystyrene support starting from the carboxyl terminal residue and adding amino acids in a stepwise fashion until the entire peptide has been formed. The following references are guides to the chemistry employed during synthesis: Schnolzer et al, Int. J. Peptide Protein Res., 40: 180-193 (1992); Merrifield, J. Amer. Chem. Soc., Vol. 85, pg. 2149 (1963); Kent et al., pg 185, in Peptides 1984, Ragnarsson, Ed. (Almquist and Weksell, Stockholm, 1984); Kent et at., pg. 217 in Peptide Chemistry 84, Izumiya, Ed. (Protein Research Foundation, B. H. Osaka, 1985); Merrifield, Science, Vol. 232, pgs. 341-347 (1986); Kent, Ann. Rev. Biochem., Vol. 57, pgs. 957-989 (1988), and references cited in these latter two references.

Preferably, chemical synthesis of polypeptides of the invention is carried out by the assembly of peptide fragments by native chemical ligation, as described by Dawson et al, Science, 266: 776-779 (1994) and Kent el al, U.S. Pat. No. 6,184,344. Briefly, in the approach a first peptide fragment is provided with an N-terminal cysteine having an unoxidized sulfhydryl side chain, and a second peptide fragment is provided with a C-terminal thioester. The unoxidized sulfhydryl side chain of the N-terminal cysteine is then condensed with the C-terminal thioester to produce an intermediate peptide fragment which links the first and second peptide fragments with a β-aminothioester bond. The β-aminothioester bond of the intermediate peptide fragment then undergoes an intramolecular rearrangement to produce the peptide fragment product which links the first and second peptide fragments with an amide bond. Preferably, the N-terminal cysteines of internal fragments are protected from undesired cyclization and/or concatenation reactions by a cyclic thiazolidine protecting group as described below. Preferably, such cyclic thiazolidine protecting group is a thioprolinyl group.

Peptide fragments having a C-terminal thioester may be produced as described in the following references, which are incorporated by reference: Kent et al, U.S. Pat. No. 6,184,344; Tarn et al, Proc. Natl. Acad. Sci., 92: 12485-12489 (1995); Blake, Int. J. Peptide Protein Res., 17: 273 (1981); Canne et al, Tetrahedron Letters, 36: 1217-1220 (1995); Hackeng et al, Proc. Natl. Acad. Sci., 94: 7845-7850 (1997); or Hackeng et al, Proc. Natl. Acad. Sci., 96: 10068-10073 (1999). Preferably, the method described by Hackeng et al (1999) is employed. Briefly, peptide fragments are synthesized on a solid phase support (described below) typically on a 0.25 mmol scale by using the in situ neutralization/HBTU activation procedure for Boc chemistry disclosed by Schnolzer et al, Int. J. Peptide Protein Res., 40: 180-193 (1992), which reference is incorporated herein by reference. (HBTU is 2-(1H-benzotriazol-1-yl)-1,1,3,3-tetramethyluronium hexafluorophosphate and Boc is tert-butoxycarbonyl). Each synthetic cycle consists of N^{α}-Boc removal by a 1- to 2-minute treatment with neat TFA, a 1-minute DMF flow wash, a 10- to 20-minute coupling time with 1.0 mmol of preactivated Boc-amino acid in the presence of DIEA, and a second DMF flow wash. (TFA is trifluoroacetic acid, DMF is N,N-dimethylformamide, and DIEA is N,N-diisopropylethylamine). N^{α}-Boc-amino acids (1.1 mmol) are preactivated for 3 minutes with 1.0 nunol of HBTU (0.5 M in DMF) in the presence of excess DIEA (3 mmol). After each coupling step, yields are determined by measuring residual free amine with a conventional quantitative ninhydrin assay, e.g. as disclosed in Sarin et al, Anal. Biochem., 117: 147-157 (1981). After coupling of Gln residues, a DCM flow wash is used before and after deprotection by using TFA, to prevent possible high-temperature (TFA/DMF)-catalyzed pyrrolidone formation. After chain assembly is completed, the peptide fragments are deprotected and cleaved from the resin by treatment with anhydrous HF for 1 hour at 0° C. with 4% p-cresol as a scavenger. The imidazole side-chain 2,4-dinitrophenyl (dnp) protecting groups remain on the His residues because the dnp-removal procedure is incompatible with C-terminal thioester groups. However, dnp is gradually removed by thiols during the ligation reaction. After cleavage, peptide fragments are precipitated with ice-cold diethylether, dissolved in aqueous acetonitrile, and lyophilized.

Thioester peptide fragments described above are preferably synthesized on a trityl-associated mercaptopropionic acid-leucine (TAMPAL) resin, made as disclosed by Hackeng et al (1999), or comparable protocol. Briefly, N^{α}-Boc-Leu (4 mmol) is activated with 3.6 mmol of HBTU in the presence of 6 mmol of DIEA and coupled for 16 minutes to 2 mmol of p-methylbenzhydrylamine (MBHA) resin, or the equivalent. Next, 3 mmol of S-trityl mercaptopropionic acid is activated with 2.7 mmol of HBTU in the presence of 6 mmol of DIEA and coupled for 16 minutes to Leu-MBHA resin. The resulting TAMPAL resin can be used as a starting resin for polypeptide-chain assembly after removal of the trityl protecting group with two 1-minute treatments with 3.5% triisopropylsilane and 2.5% H₂O in TFA. The thioester bond can be formed with any desired amino acid by using standard in situ-neutralization peptide coupling protocols for 1 hour, as disclosed in Schnolzer et al (cited above). Treatment of the final peptide fragment with anhydrous HF yields the. C-terminal activated mercaptopropionic acid-leucine (MPAL) thioester peptide fragments.

Preferably, thiazolidine-protected thioester peptide fragment intermediates are used in native chemical ligation under conditions as described by Hackeng et al (1999), or like conditions. Briefly, 0.1 M phosphate buffer (pH 8.5) containing 6 M guanidine,. 4% (vol/vol) benzylmercaptan, and 4% (vol/vol) thiophenol is added to dry peptides to be ligated, to give a final peptide concentration of 1-3 mM at about pH 7, lowered because of the addition of thiols and TFA from the lyophilized peptide. Preferably, the ligation reaction is performed in a heating block at 37° C. and is periodically vortexed to equilibrate the thiol additives. The reaction may be monitored for degree of completion by MALDI-MS or HPLC and electrospray ionization MS.

After a native chemical ligation reaction is completed or stopped, the N-terminal thiazolidine ring of the product is opened by treatment with a cysteine deprotecting agent, such as O-methylhydroxylamine (0.5 M) at pH 3.5-4.5 for 2 hours at 37° C, after which a 10-fold excess of Tris-(2-carboxyethyl)-phosphine is added to the reaction mixture to completely reduce any oxidizing reaction constituents prior to purification of the product by conventional preparative HPLC. Preferably, fractions containing the ligation product are identified by electrospray MS, are pooled, and lyophilized.

After the synthesis is completed and the final product purified, the final polypeptide product may be refolded by conventional techniques, e.g. Creighton, Meth. Enzymol., 107: 305-329 (1984); White, Meth. Enzymol., 11: 481-484 (1967); Wetlaufer, Meth. Enzymol., 107: 301-304 (1984); and the like. Preferably, a final product is refolded by air oxidation by the following, or like: The reduced lyophilized product is dissolved (at about 0.1 mg/mL) in 1 M guanidine hydrochloride (or like chaotropic agent) with 100 mM Tris, 10 mM methionine, at pH 8.6. After gentle overnight stirring, the re-folded product is isolated by reverse phase HPLC with conventional protocols.

Recombinant Expression Vectors and Host Cells

The polynucleotide sequences described herein can be used in recombinant DNA molecules that direct the expression of the corresponding polypeptides in appropriate host cells. Because of the degeneracy in the genetic code, other DNA sequences may encode the equivalent amino acid sequence, and may be used to clone and express the Pep356-related peptides. Codons preferred by a particular host-cell may be selected and substituted into the naturally occurring nucleotide sequences, to increase the rate and/or efficiency of expression. The nucleic acid (e.g., cDNA or genomic DNA) encoding the desired Pep356-related peptide may be inserted into a replicable vector for cloning (amplification of the DNA), or for expression. The polypeptide can be expressed recombinantly in any of a number of expression systems according to methods known in the art (Ausubel, et al., editors, Current Protocols in Molecular Biology, John Wiley & Sons, New York, 3990). Appropriate host cells include yeast, bacteria, archebacteria, fungi, and insect and animal cells, including mammalian cells, for example primary cells, including stem cells, including, but not limited to bone marrow stem cells. More specifically, these include, but are not limited to, microorganisms such as bacteria transformed with recombinant bacteriophage, plasmid or cosmid DNA expression vectors, and yeast transformed with yeast expression vectors. Also included, are insect cells infected with a recombinant insect virus (such as baculovirus), and mammalian expression systems. The nucleic acid sequence to be expressed may be inserted into the vector by a variety of procedures. In general, DNA is inserted into an appropriate restriction endonuclease site using techniques known in the art. Vector components generally include, but are not limited to, one or more of a signal sequence, an origin of replication, one or more marker genes, an enhancer element, a promoter, and a transcription termination sequence. Construction of suitable vectors containing one or more of these components employs standard ligation techniques known to the skilled artisan.

The Pep356-related peptides of the present invention are produced by culturing a host cell transformed with an expression vector containing a nucleic acid encoding a Pep356-related peptide, under the appropriate conditions to induce or cause expression of the protein. The conditions appropriate for Pep356-related peptide expression will vary with the choice of the expression vector and the host cell, and will be easily ascertained by one skilled in the art through routine experimentation. For example, the use of constitutive promoters in the expression vector will require optimizing the growth and proliferation of the host cell, while the use of an inducible promoter requires the appropriate growth conditions for induction. In addition, in some embodiments, the timing of the harvest is important. For example, the baculoviral systems used in insect cell expression are lytic viruses, and thus harvest time selection can be crucial for product yield.

A host cell strain may be chosen for its ability to modulate the ekpression of the inserted sequences or to process the expressed protein in the desired fashion. Such modifications of the protein include, but are not limited to, acetylation, carboxylation, glycosylation, phosphorylation, lipidation and acylation. Post-translational processing, which cleaves a “prepro” form of the protein, may also be important for correct insertion, folding and/or function. By way of example, host cells such as CHO, HeLa, BHK, MDCK, 293, W138, etc. have specific cellular machinery and characteristic mechanisms for such post-translational activities and may be chosen to ensure the correct modification and processing of the introduced, foreign protein. Of particular interest are Drosophila melanogaster cells, Saccharomyces cerevisiae and other yeasts, E. coli, Bacillus subtilis, SF9 cells, C129 cells, 293 cells, Neurospora, BHK, CHO, COS, and HeLa cells, fibroblasts, Schwanoma cell lines, immortalized mammalian myeloid and lymphoid cell lines, Jukat cells, human cells and other primary cells.

The nucleic acid encoding a Pep356-related peptide must be “operably linked” by placing it into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” DNA sequences are contiguous, and, in the case of a secretory leader, contiguous and in reading phase. However, enhancers do not have to be contiguous. Linking is accomplished by ligation at convenient restriction sites. If such sites do not exist, the synthetic oligonucleotide adaptors or linkers are used in accordance with conventional practice. Promoter sequences encode either constitutive or inducible promoters. The promoters may be either naturally occurring promoters or hybrid promoters. Hybrid promoters, which combine elements of more than one promoter, are also known in the art, and are useful in the present invention. The expression vector may comprise additional elements, for example, the expression vector may have two replication systems, thus allowing it to be maintained in two organisms, for example in mammalian or insect cells for expression and in a prokaryotic host for cloning and amplification. Both expression and cloning vectors contain a nucleic acid sequence that enables the vector to replicate in one or more selected host cells. Such sequences are well known for a variety of bacteria, yeast, and viruses. The origin of replication from the plasmid pBR322 is suitable for most Gram-negative bacteria, the 2: plasmid origin is suitable for yeast, and various viral origins (SV40, polyoma, adenovirus, VSV or BPV) are useful for cloning vectors in mammalian cells. Further, for integrating expression vectors, the expression vector contains at least one sequence homologous to the host cell genome, and preferably, two homologous sequences which flank the expression construct. The integrating vector may be directed to a specific locus in the host cell by selecting the appropriate homologous sequence for inclusion in the vector. Constructs for integrating vectors are well known in the art.

Preferably, the expression vector contains a selectable marker gene to allow the selection of transformed host cells. Selection genes are well known in the art and will vary with the host cell used. Expression and cloning vectors will typically contain a selection gene, also termed a selectable marker. Typical selection genes encode proteins that (a) confer resistance to antibiotics or other toxins, e.g., ampicillin, neomycin, methotrexate, or tetracycline, (b) complement auxotrophic deficiencies, or (c) supply critical nutrients not available for from complex media, e.g., the gene encoding D-alanine racemase for Bacilli.

Host cells transformed with a nucleotide sequence encoding a Pep356-related peptide may be cultured under conditions suitable for the expression and recovery of the encoded protein from cell culture. The protein produced by a recombinant cell may be secreted, membrane-bound, or contained intracellularly depending on the sequence and/or the vector used. As will be understood by those of skill in the art, expression vectors containing polynucleotides encoding the Pep356-related peptides can be designed with signal sequences which direct secretion of the Pep356-related peptides through a prokaryotic or eukaryotic cell membrane. The desired Pep356-related peptide may be produced recombinantly not only directly, but also as a fusion polypeptide with a heterologous polypeptide, which may be a signal sequence or other polypeptide having a specific cleavage site at the N-terminus of the mature protein or polypeptide. In general, the signal sequence may be a component of the vector, or it may be a part of the Pep356-related peptides-encoding DNA that is inserted into the vector. The signal sequence may be a prokaryotic signal sequence selected, for example, from the group of the alkaline phosphatase, penicillinase, lpp, or heat-stable enterotoxin II leaders. For yeast secretion the signal sequence may be, e.g., the yeast invertase leader, alpha factor leader (including Saccharomyces and Kluyveromyces a-factor leaders, the latter described in U.S. Pat. No. 5,010,182), or acid phosphatase leader, the C. albicans glucoamylase leader (EP 362,179 published Apr. 4, 1990), or the signal described in WO 90113646 published Nov. 15, 1990. In mammalian cell expression, mamrnalian signal sequences may be used to direct secretion of the protein, such as signal sequences from secreted polypeptides of the same or related species, as well as viral secretory leaders. According to the expression system selected, the coding sequence is inserted into an appropriate vector, which in turn may require the presence of certain characteristic “control elements” or “regulatory sequences.” Appropriate constructs are known generally in the art (Ausubel, et al., 1990) and, in many cases, are available from commnercial suppliers such as Invitrogen (San Diego, Calif.), Stratagene (La Jolla, Calif.), Gibco BRL (Rockville, Md.) or Clontech (Palo Alto, Calif.).

Expression in Bacterial Systems

Transformation of bacterial cells may be achieved using an inducible promoter such as the hybrid lacZ promoter of the “BLUESCRIPT” Phagemid (Stratagene) or “pSPORT1” (Gibco BRL). In addition, a number of expression vectors may be selected for use in bacterial cells to produce cleavable fusion proteins that can be easily detected and/or purified, including, but not limited to “BLUESCRIPT” (a-galactosidase; Stratagene) or pGEX (glutathione S-transferase; Promega, Madison, Wis.). A suitable bacterial promoter is any nucleic acid sequence capable of binding bacterial RNA polymerase and initiating the downstream (3′) transcription of the coding sequence of the Pep356 gene into mRNA. A bacterial promoter has a transcription initiation region which is usually placed proximal to the 5′ end of the coding sequence. This transcription initiation region typically includes an RNA polymerase binding site and a transcription initiation site. Sequences encoding metabolic pathway enzymes provide particularly useful promoter sequences. Examples include promoter sequences derived from sugar metabolizing enzymes, such as galactose, lactose and maltose, and sequences derived from biosynthetic enzymes such as tryptophan. Promoters from bacteriophage may also be used and are known in the art. In addition, synthetic promoters and hybrid promoters are also useful; for example, the tat promoter is a hybrid of the trp and lac promoter sequences. Furthermore, a bacterial promoter can include naturally occurring promoters of non-bacterial origin that have the ability to bind bacterial RNA polymerase and initiate transcription. An efficient ribosome binding site is also desirable. The expression vector may also include a signal peptide sequence that provides for secretion of the Pep356-related peptide in bacteria. The signal sequence typically encodes a signal peptide comprised of hydrophobic amino acids which direct the secretion of the protein from the cell. The protein is either secreted into the growth media (gram-positive bacteria) or into the periplasmic space, located between the inner and outer membrane of the cell (gram-negative bacteria). The bacterial expression vector may also include a selectable marker gene to allow for the selection of bacterial strains that have been transformed. Suitable selection genes include drug resistance genes such as anipicillin, chloramphenicol, erythromycin, kanamycin, neomycin and tetracycline. Selectable markers also include biosynthetic genes, such as those in the histidine, tryptophan and leucine biosynthetic pathways. When large quantities of Pep356-related peptides are needed, e.g., for the induction of antibodies, vectors which direct high level expression of fusion proteins that are readily purified may be desirable. Such vectors include, but are not limited to, multifunctional E. coli cloning and expression vectors such as BLUESCRIPT (Stratagene), in which the Pep356-related peptide coding sequence may be ligated into the vector in-frame with sequences for the amino-tenninal Met and tie subsequent 7 residues of beta-galactosidase so that a hybrid protein is produced; PIN vectors (Van Heeke & Schuster J Biol Chem 264:5503-5509 1989)); PET vectors (Novagen, Madison Wis.). Expression vectors for bacteria include the various components set forth above, and are well known in the art. Examples include vectors for Bacillus subtilis, E. coli, Streptococcus cremoris, and Streptococcus lividans. Bacterial expression vectors are transformed into bacterial host cells using techniques well known in the art, such as calcium chloride mediated transfection or electroporation.

Expression in Yeast

Yeast expression systems are well known in the art, and include expression vectors for Saccharomyces cerevisiae, Candida albicans and C. maltosa, Hansenula polymorpha, Kluyveromyces fragilis and K. lactis, Pichia guiillermondii and P pastoris, Schizosaccharomyces pombe, and Yarrowia lipolytica. Examples of suitable promoters for use in yeast hosts include the promoters for 3-phosphoglycerate kinase (Hitzeman et al., J. Biol. Chem. 255:2073 (1980)) or other glycolytic enzymes (Hess et al., J. Adv. Enzyme Reg. 7:149 (1968); Holland, Biochemistry 17:4900 (1978)), such as enolase, glyceraldehyde-3-phosphate dehydrogenase, hexokinase, pyruvate decarboxylase, phosphofructokinase, glucose-6-phosphate isomerase, 3-phosphoglycerate mutase, pyruvate kinase, triosephosphate isomerase, phosphoglucose isomerase, alpha factor, the ADH2IGAPDH promoter, glucokinase alcohol oxidase, and PGH (Ausubel, et al., 1990; Grant et al., Methods in Enzymology 153:516-544, (1987)). Other yeast promoters, which are inducible have the additional advantage of transcription controlled by growth conditions, include the promoter regions for alcohol dehydrogenase 2, isocytochrome C, acid phosphatase, degradative enzymes associated with nitrogen metabolism, metallothionein, glyceraldehyde-3-phosphate dehydrogenase, and enzymes responsible for maltose and galactose utilization. Suitable vectors and promoters for use in yeast expression are further described in EP 73,657. Yeast selectable markers include ADE2. HIS4. LEU2. TRP1. and ALG7, which confers resistance to tunicamycin; the neomycin phosphotransferase gene, which confers resistance to G418; and the CUP 1 gene, which allows yeast to grow in the presence of copper ions. Yeast expression vectors can be constructed for intracellular production or secretion of a Pep356-related peptide from the DNA encoding the Pep356-related peptides of interest. For example, a selected signal peptide and the appropriate constitutive or inducible promoter may be inserted into suitable restriction sites in the selected plasmid for direct intracellular expression of the Pep356-related peptide. For secretion of the Pep356-related peptides, DNA encoding the Pep356-related peptide can be cloned into the selected plasmid, together with DNA encoding the promoter, the yeast alpha-factor secretory signal/leader sequence, and linker sequences (as needed), for expression of the Pep356-related peptide. Yeast cells, can then be transformed with the expression plasmids described above, and cultured in an appropriate fermentation media. The protein produced by such transformed yeast can then be concentrated by precipitation with 10% trichloroacetic acid and analyzed following separation by SDS-PA GE and staining of the gels with Coomassie Blue stain. The recombinant Pep356-related peptides can subsequently be isolated and purified from the fermentation medium by techniques known to those of skill in the art.

Expression in Mammalian Systems

The Pep356-related peptides may be expressed in mammalian cells. Mammalian expression systems are known in the art, and include retroviral vector mediated expression systems. Mammalian host cells may be transformed with any of a number of different viral-based expression systems, such as adenovirus, where the coding region can be ligated into an adenovirus transcription/translation complex consisting of the late promoter and tripartite leader sequence. Insertion in a nonessential E1 or E3 region of the viral genome results in a viable virus capable of expression of the polypeptide of interest in infected host cells. A preferred expression vector system is a retroviral vector system such as is generally described in PCT/US97/01019 and PCT/US97/101048. Suitable mammalian expression vectors contain a mammalian promoter which is any DNA sequence capable of binding mammalian RNA polymerase and initiating the downstream (3′) transcription of a coding sequence for Pep356-related peptide into mRNA. A promoter will have a transcription initiating region, which is usually placed proximal to the 5′ end of the coding sequence, and a TATA box, using a located 25-30 base pairs upstream of the transcription initiation site. The TATA box is thought to direct RNA polymerase II to begin RNA synthesis at the correct site. A mammalian promoter will also contain an upstream promoter element (enhancer element), typically located within 100 to 200 base pairs upstream of the TATA box. An upstream promoter element determines the rate at which transcription is initiated and can act in either orientation. Of particular use as mammalian promoters are the promoters from mammalian viral genes, since the viral genes are often highly expressed and have a broad host range. Examples include promoters obtained from the genomes of viruses such as polyoma virus, fowlpox virus (UK 2,211, 504 published Jul. 5, 1989), adenovirus (such as Adenovirus 2), bovine papilloma virus, avian sarcoma virus, cytomegalovirus, a retrovirus, hepatitis-B virus and Simian Virus 40 (SV40), from heterologous mammalian promoters, e.g., the actin promoter or an immunoglobulin promoter, and from heat-shock promoters, provided such promoters are compatible with the host cell systems. Transcription of a DNA encoding a Pep356-related peptide by higher eukaryotes may be increased by inserting an enhancer sequence into the vector. Enhancers are cis-acting elements of DNA, usually about from 10 to 300 bp, that act on a promoter to increase its transcription. Many enhancer sequences are now known from mammalian genes (globin, elastase, albumin, a-fetoprotein, and insulin). Typically, however, one will use an enhancer from a eukaryotic cell virus. Examples include the SV40 enhancer, the cytomegalovirus early promoter enhancer, the polyoma enhancer on the late side of the replication origin, and adenovirus enhancers. The enhancer is preferably located at a site 5′ from the promoter. In general, the transcription termination and polyadenylation sequences recognized by mammalian cells are regulatory regions located 3′ to the translation stop codon and thus, together with the promoter elements, flank the coding sequence. The 3′ terminus of the mature mRNA is formed by site-specific post-translational cleavage and polyadenylation. Examples of transcription terminator and pglyadenylation signals include those derived from SV40. Long term, high-yield production of recombinant proteins can be effected in a stable expression systemn. Expression vectors which contain viral origins of replication or endogenous expression elements and a selectable marker gene may be used for this purpose. Appropriate vectors containing selectable markers for use in mammalian cells are readily available commercially and are known to persons skilled in the art. Examples of such selectable markers include, but are not limited to herpes simplex virus thymi-dine kinase and adenine phosphoribosyltransferase for use in tk- or hprt-cells, respectively. The methods of introducing exogenous nucleic acid into mammalian hosts, as well as other hosts, is well known in the art, and will vary with the host cell used. Techniques include dextran-mediated transfection, calcium phosphate precipitation, polybrene mediated transfection, protoplast fusion, electroporation, viral infection, encapsulation of the polynucleotide(s) in liposomes, and direct microinjection of the DNA into nuclei.

Pep356-related peptides can be purified from culture supernatants of mammalian cells transiently transfected or stably transfonned by an expression vector carrying a Pep356-related peptide-encoding sequence. Preferably, Pep356-related peptides are purified from culture supernatants of COS 7 cells transiently transfected by the pcD expression vector. Transfection of COS 7 cells with pcD proceeds as follows: One day prior to transfection, approximately 10⁶ COS 7 monkey cells are seeded onto individual 100 mm plates in Dulbecco's modified Eagle medium (DME) containing 10% fetal calf serum and 2 mM glutamine. To perform the transfection, the medium is aspirated from each plate and replaced with 4 ml of DME containing 50 mM Tris.HCl pH 7.4, 400 mg/ml DEAE-Dextran and 50 μg of plasmid DNA. The plates are incubated for four liours at 37° C., then the DNA-containing medium is removed, and the plates are washed twice with 5 ml of serum-free DME. DME is added back to the plates which are then incubated for an additional 3 hrs at 37° C. The plates are washed once with DME, after which DME containing 4% fetal calf serum, 2 mM glutamine, penicillin (100 U/L) and streptomycin (100 μg/L) at standard concentrations is added. The cells are then incubated for 72 hrs at 37° C., after which the growth medium is collected for purification of Pep356-related peptides. Plasmid DNA for the transfections is obtained by growing pcD(SRα), or like expression vector, containing the Pep356-related peptide-encoding cDNA insert in E. coli MC 1061, described by Casadaban and Cohen, J. Mol. Biol., Vol. 138, pgs. 179-207 (1980), or like organism. The plasmid DNA is isolated from the cultures by standard techniques, e.g. Sambrook et al., Molecular Cloning: A Laboratory Manual, Second Edition (Cold Spring Harbor Laboratory, New York, 1989) or Ausubel et al (1990, cited above).

Expression in Insect Cells

Pep356-related peptides may also be produced in insect cells. Expression vectors for the transformation of insect cells, and in particular, baculovirus-based expression vectors, are well known in the art. In one such system, the Pep356-related peptides-encoding DNA is fused upstream of an epitope tag contained within a baculovirus expression vector. Autographa californica nuclear polyhedrosis virus (AcNPV) is used as a vector to express foreign genes in Spodoptera frugiperda Sf9 cells or in Trichoplusia larvae. The Pep356-related peptides-encoding sequence is cloned into a nonessential region of the virus, such as the polyhedrin gene, and placed under control of the polyhedrin promoter. Successful insertion of a Pep356-related peptides-encoding sequence will render the polyhedrin gene inactive and produce recombinant virus lacking coat protein coat. The recombinant viruses are then used to infect Sf9 cells or Trichoplusia larvae in which the Pep356-related peptides is expressed (Smith et al., J. Wol. 46:584 (1994); Engelhard E K et al., Proc. Nat. Acad. Sci. 91:3224-3227 (1994)). Suitable epitope tags for fusion to the Pep356-related peptides-encoding DNA include poly-his tags and immunoglobulin tags (like Fc regions of IgG). A variety of plasmids may be employed, including commercially available plasmids such as pVL1393 (Novagen). Briefly, the Pep356-related peptides-encoding DNA or the desired portion of the Pep356-related peptides-encoding DNA is amplified by PCR with primers complementary to the 5′ and 3′ regions. The 5′ primer may incorporate flanking restriction sites. The PCR product is then digested with the selected restriction enzymes and subcloned into an expression vector. Recombinant baculovirus is generated by co-transfecting the above plasmid and BaculoGold™ virus DNA (Pharmingen) into Sf9 cells (ATCC CRL 1711) using lipofectin (commercially available from GIBCO-BRL), or other methods known to those of skill in the art. Virus is produced by day 4-5 of culture in Sf9 cells at 28° C., and used for further amplifications. Procedures are performed as further described in O'Reilley et al., BACULOVIRUS EXPRESSION VECTORS: A LABORATORY MANUAL, Oxford University Press (1994). Extracts may be prepared from recombinant virus-infected Sf9 cells as described in Rupert et al., Nature 362:175-179 (1993). Alternatively, expressed epitope-tagged Pep356-related peptides can be purified by affinity chromatography, or for example, purification of an IgG tagged (or Fc tagged) Pep356-related peptide can be performed using chromatography techniques, including Protein A or protein G column chromatography.

Evaluation of Gene Expression

Gene expression may be evaluated in a sample directly, for example, by standard techniques known to those of skill in the art, e.g., Southern blotting for DNA detection, Northern blotting to determine the transcription of mRNA, dot blotting (DNA or RNA), or in situ hybridization, using an appropriately labeled probe, based on the sequences provided herein. Altematively, antibodies may be used in assays for detection of polypeptides, nucleic acids, such as specific duplexes, including DNA duplexes, RNA duplexes, and DNA-RNA hybrid duplexes or DNA-protein duplexes. Such antibodies may be labeled and the assay carried out where the duplex is bound to a surface, so that upon the formation of duplex on the surface, the presence of antibody bound tothe duplex can be detected. Gene expression, alternatively, may be measured by immunohistochemical staining of cells or tissue sections and assay of cell culture or body fluids, to directly evaluate the expression of Pep356-related peptides. Antibodies useful for such inmmunological assays may be either monoclonal or polyclonal, and may be prepared against a native sequence Pep356-related peptides. Protein levels may also be detected by mass spectrometry. A further method of protein detection is with protein chips.

Purification of Expressed Protein

Expressed Pep356-related peptides may be purified or isolated after expression, using any of a variety of methods known to those skilled in the art. The appropriate technique will vary depending upon what other components are present in the sample. Contaminant components that are removed by isolation or purification are materials that would typically interfere with diagnostic or therapeutic uses for the polypeptide, and may include enzymes, hormones, and other solutes. The purification step(s) selected will depend, for example, on the nature of the production process used and the particular Pep356-related peptide produced. A Pep356-related peptide or protein may be recovered from culture medium or from host cell lysates. If membrane-bound, it can be released from the membrane using a suitable detergent solution (e.g. Triton-X 100) or by enzymatic cleavage. Alternatively, cells employed in expression of Pep356-related peptides can be disrupted by various physical or chemical means, such as freeze-thaw cycling, sonication, mechanical disruption, or by use of cell lysing agents. Exemplary purification methods include, but are not limited to, ion-exchange column chromatography; chromatography using silica gel or a cation-exchange resin such as DEAE; gel filtration using, for example, Sephadex G-75; protein A Sepharose columns to remove contaminants such as IgG; chromatography using metal chelating columns to bind epitope-tagged forms of the Pep356-related peptide; ethanol precipitation; reverse phase HPLC; chromatofocusing; SDS-PA GE; and ammonium sulfate precipitation. Ordinarily, an isolated Pep356-related peptide will be prepared by at least one purification step. For example, the Pep356-related peptide may be purified using a standard anti-Pep356-related antibody column. Ultrafiltration and dialysis techniques, in conjunction with protein concentration, are also useful (see, for example, Scopes, R., PROTEIN PURIFICATION, Springer-Verlag, New York, N.Y., 1982). The degree of purification necessary will vary depending on the use of the Pep356-related peptides. In some instances no purification will be necessary. Once expressed and purified as needed, the Pep356-related peptides and nucleic acids of the present invention are useful in a number of applications, as detailed below.

Transgenic Animals

The host cells of the invention can also be used to produce nonhuman transgenic animals. For example, in one embodiment, a host cell of the invention is a fertilized oocyte or an embryonic stem cell into which Pep356-related peptides-coding sequences have been introduced. Such host cells can then be used to create non-human transgenic animals in which exogenous Pep356-related peptides sequences have been introduced into their genome or homologous recombinant animals in which endogenous Pep356-related peptides sequences have been altered. Such animals are useful for studying the function and/or activity of a Pep356-related peptide or fragment thereof and for identifying and/or evaluating modulators of Pep356-related activity. As used herein, a “transgenic animal” is a non-human animal, preferably a mammal, more preferably a rodent such as a rat or mouse, in which one or more of the cells of the animal includes a transgene. Other examples of transgenic animals include non-human primates, sheep, dogs, cows, goats, chickens, amphibians, etc. A transgene is exogenous DNA which is integrated into the genome of a cell from which a transgenic animal develops and which remains in the genome of the mature animal, thereby directing the expression of an encoded gene product in one or more cell types or tissues of the transgenic animaL As used herein, a “homologous recombinant animal” is a non-human animal, preferably a mammal, more preferably a mouse, in which an endogenous Pep356 gene has been altered by homologous recombination between the endogenous gene and an exogenous DNA molecule introduced into a cell of the animal, e.g., an embryonic cell of the animal, prior to development of the animal.

A transgenic animal of the invention can be created by introducing a Pep356-related peptides-encoding nucleic acid into the male pronuclei of a fertilized oocyte, e.g., by microinjection or retroviral infection, and allowing the oocyte to develop in a pseudopregnant female foster animal. The Pep356-related peptides cDNA sequence or a fragment thereof can be introduced as a transgene into the genome of a non-human animal. Alternatively, a nonhuman homologue of a human Pep356 gene. such as a mouse or rat Pep356 gene, can be used as a transgene. Intronic sequences and polyadenylation signals can also be included in the transgene to increase the efficiency of expression of the transgene. A tissue-specific regulatory sequence(s) can be operably linked to a Pep356-related peptides transgene to direct expression of a Pep356-related peptide to particular cells. Methods for generating transgenic animals via embryo manipulation and microinjection, particularly animals such as mice, have become conventional in the art and are described, for example, in U.S. Pat. Nos. 4,736,866 and 4,870,009, both by Leder et al., U.S. Pat. No. 4,873,191 by Wagner et al. and in Hogan, B., Manipulating the Mouse Embryo, (Cold Spring Harbor Laboratory Press, Cold Spring Harbor, N.Y., 1986). Similar methods are used for production of other transgenic animals. A transgenic founder animal can be identified based upon the presence of a Pep356-related peptides transgene in its genome and/or expression of Pep356-related peptides mRNA in tissues or cells of the animals. A transgenic founder animal can then be used to breed additional animals carrying the transgene. Moreover, transgenic animals carrying a transgene encoding a Pep356-related peptide can further be bred to other transgenic animals carrying other transgenes.

To create an animal in which a desired nucleic acid has been introduced into the genome via homologous recombination, a vector is prepared which contains at least a portion of a Pep356 gene into which a deletion, addition or substitution has been introduced to thereby alter, e.g., functionally disrupt, the Pep356 gene. The Pep356 gene can be a human gene, but more preferably, is a non-human homologue of a human Pep356 gene (e.g., a cDNA isolated by stringent hybridization with a nucleotide sequence coding for a Pep356-related peptide). For example, a mouse Pep356 gene can be used to construct a homologous recombination vector suitable for altering an endogenous gene in the mouse genome. In a preferred embodiment, the vector is designed such that, upon homologous recombination, the endogenous Pep356 gene is functionally disrupted (i.e., no longer encodes a functional protein; also referred to as a “knock out” vector). Alternatively, the vector can be designed such that, upon homologous recombination, the endogenous Pep356 gene is mutated or otherwise altered but still encodes functional protein (e.g., the upstream regulatory region can be altered to thereby alter the expression of the endogenous Pep356-related peptide). In the homologous recombination vector, the altered portion of the Pep356-related peptides or gene is flanked at its 5′ and 3′ ends by additional nucleic acid sequence of the Pep356 gene to allow for homologous recombination to occur between the exogenous Pep356 gene carried by the vector and an endogenous Pep356 gene in an embryonic stem cell. The additional flanking Pep356-related nucleic acid sequence is of sufficient length for successful homologous recombination with the endogenous gene. Typically, several kilobases of flanking DNA (both at the 5′ and 3′ ends) are included in the vector (see e.g., Thomas, K. R. and Capecchi, M. R. (1987) Cell 51:503, the disclosure of which is incorporated herein by reference in its entirety, for a description of homologous recombination vectors). The vector is introduced into an embryonic stem cell line (e.g., by electroporation) and cells in wvhich the introduced Pep356 gene has homologously recombined with the endogenous Pep356 gene are selected (see e.g., Li, E. et al. (1992) Cell 69:915, the disclosure of which is incorporated herein by reference in its entirety). The selected cells are then injected into a blastocyst of an animal (e.g., a mouse) to form aggregation chimeras (see e.g., Bradley, A. in Teratocarcinomas and Embryonic Stem Cells. A Practical Approach, E. J. Robertson, ed. (IRL, Oxford, 1987) pp. 113-152). A chimeric embryo can then be implanted into a suitable pseudopregnant female foster animal and the embryo brought to term. Progeny harboring the homologously recombined DNA in their germ cells can be used to breed animals in which all cells of the animal contain the homologously recombined DNA by germline transmission of the transgene. Methods for constructing homologous recombination vectors and homologous recombinant animals are described further in Bradley, A. (1991) Current Opinion in Biotechnology 2:823-829 and in WO 90/11354 by Le Mouellec et al.; WO 91/01140 by Smithies et al. WO 92/0968 by Zijlstra et al.; and WO 93/04169 by Berns et al.

In another embodiment, transgenic non-human animals can be produced which contain selected systems which allow for regulated expression of the transgene. One example of such a system is the cre/loxP recombinase system of bacteriophage P1. For a description of the cre/loxP recombinase system, see, e.g., Lakso et al. (1992) PNAS 89:6232-6236. Another example of a recombinase system is the FLP recombinase system of Saccharomyces cerevisiae (O'Gorman et al. (1991) Science 251:135 1-1355). If a cre/loxP recombinase system is used to regulate expression of the transgene, animals containing transgenes encoding both the Cre recombinase and a selected protein are required. Such animals can be provided through the construction of “double” transgenic animals, e.g., by mating two transgenic animals, one containing a transgene encoding a selected protein and the other containing a transgene encoding a recombinase.

Assessing Polypeptides Activity

It will be appreciated that the invention further provides methods of testing the activity of, or obtaining, functional fragments and variants of Pep356-related peptides and Pep356-related nucleotide sequences involving providing a variant or modified Pep356-related peptides, or Pep356-related nucleic acid and assessing whether a polypeptide encoded thereby displays a Pep356-related activity of the invention. Encompassed is thus a method of assessing the finction of a Pep356-related peptides polypeptide comprising: (a) providing a Pep356-related peptide, or a biologically active fragment or homologue thereof; and (b) testing said Pep356-related peptide, or a biologically active fragment or homologue thereof for a Pep356-related activity, preferably a Pep356-related activity. Any suitable formnat may be used, including cell free, cell-based and in vivo formnats. For example, said assay may comprise expressing a Pep356-related nucleic acid in a host cell, and observing Pep356-related activity in said cell. In another example, a Pep356-related peptide, or a biologically active fragment or homologue thereof is introduced to a cell, and a Pep356-related activity is observed.

A Pep356-related activity may be any activity as described herein, including: (1) antigenicity, or the ability to bind an anti-Pep356-related peptide specific antibody; (2) immunogenicity, or the ability to generate an anti-Pep356-related peptide specific antibody; (3) interacting with a Pep356-related target molecule, preferably a protein, in particular, a microfibril associated protease such as elastase; (4) circulating in human plasma; (5) mediating.an angiogenesis, particularly during lung development and wound healing; and (6) undergoing dibasic cleavage.

Pep356-related biological activity can be assayed by any suitable method known in the art. Antigenicity and immunogenicity may be detected, for example, as described in the sections titled “Anti Pep3S6-related peptide antibodies” and “Uses of Pep356-related peptide antibodies.” Circulation in blood plasma may be detected as described in “Diagnostic and Prognostic Uses.” Interaction with a Pep356-related peptide target molecule may be detected according to any of the methods described herein, for example, in the section titled “Drug Screening Assays.” Detection of dibasic cleavage may be accomplished using known SDS-PA GE or mass spectrometry techniques.

An in vivo angiogenesis assay is described in U.S. Pat. No. 5,382,514, and a mouse model of hind limb ischernia was described by Couffinhal et al., 1998, Am. J. Pathol. 152:1667-1679. Direct effects of angiogenic molecules on vascular wall cells may also be detected by in vitro assays. Such assays facilitate the study of endothelial functions that are essential for new blood vessel formation. Most in vitro models of angiogenesis use extracellular matrix substrata containing growth-regulatory molecules (Vukicevic et al., 1992, Exp. Cell. Res. 202:1-8). To test the formation of capillary sprouts and endothelial cell sprouting (e.g. Koblizek et al. (1 998) Curr. Biol. 8:529-532), most assays require exogenous stimuli such as phorbol esters or angiogenic molecules to induce the formation of endothelial cords and tubes. Many protein factors, including all known angiopoeitins and growth actors, have exhibited actiivity in one or more factor dependent cell proliferation assays, and hence the assays serve as a convenient confirmation of activity. Pep356-related angiogenic activity is evidenced by any one of a number of routine factor dependent cell proliferation assays for cell lines including, without limitation, 32D, DA2, DA1 G, TI 0,B9, B9/1 1, BaF3, MC9/G, M+ (preBM+) 2E8, RB5, DA1, HT2, CTLL2, TF-1, Mo7e and CMK. Detecting Pep356-related activity may also comprise detecting any suitable therapeutic endpoint discussed herein in the section titled “Methods of Treatment”.

Anti-Pep356-related Antibodies

The present invention provides antibodies and binding compositions specific for Pep356-related peptides. Such antibodies and binding compositions include polyclonal antibodies, monoclonal antibodies, Fab and single chain Fv fragments thereof, bispecific antibodies, heteroconjugates, and humanized antibodies. Such antibodies and binding compositions may be produced in a variety of ways, including hybridoma cultures, recombinant expression in bacteria or mammalian cell cultures, and recombinant expression in transgenic animals. There is abundant guidance in the literature for selecting a particular production methodology, e.g. Chadd and Chamow, Curr. Opin. Biotechnol., 12: 188-194 (2001).

The choice of manufacturing methodology depends on several factors including the antibody structure desired, the importance of carbohydrate moieties on the antibodies, ease of culturing and purification, and cost. Many different antibody structures may be generated using standard expression technology, including full-length antibodies, antibody fragments, such as Fab and Fv fragments, as well as chimeric antibodies comprising components from different species. Antibody fragments of small size, such as Fab and Fv fragments, having no effector functions and limited pharmokinetic activity may be generated in a bacterial expression system. Single chain Fv fragments are highly selective for in vivo tumors, show good tumor penetration and low immunogenicity, and are cleared rapidly from the blood, e.g. Freyre et al, J. Biotechnol., 76: 157-163 (2000). Thus, such molecules are desirable for radioimmunodetection and in situ radiotherapy. Whencver pharmacokinetic activity in the form of increased half-life is required for therapeutic purposes, full-length antibodies are preferable. For example, immunoglobulin G (IgG) the molecule may be one of four subclasses: γ1, γ2, γ3, or γ4. If a full-length antibody with effector function is required, then IgG subclasses γ1 or γ3 are preferred, and IgG subclass γ1 is most preferred. The γ1 and γ3 subclasses exhibit potent effector function, complement activation, and promote antibody-dependent cell-mediated cytotoxicity through interaction with specific Fc receptors, e.g. Raju et al, Glycobiology, 10: 477-486 (2000); Lund et al, J. Immunol., 147: 2657-2662 (1991).

Polyclonal Antibodies

The anti-Pep356-related antibodies of the present invention may be polyclonal antibodies. Such polyclonal antibodies can be produced in a mammal, for example, follonving one or more injections of an immunizing agent, and preferably, an adjuvant. Typically, the immunizing agent and/or adjuvant will be injected into the mammal by a series of subcutaneous or intraperitoneal injections. The immunizing agent may include Pep356-related peptides or a fusion protein thereof It may be useful to conjugate the antigen to a protein known to be immunogenic in the mammal being immunized. Examples of such immunogenic proteins include, but are not limited to, keyhole limpet hemocyanin (KLH), serum albumin, bovine thyroglobulin, and soybean trypsin inhibitor. Adjuvants include, for example, Freund's complete adjuvant and MPL-TDM adjuvant (monophosphoryl Lipid A, synthetic trehalose dicoryno-mycolate). The immnunization protocol may be determined by one skilled in the art based on standard protocols.

Monoclonal Antibodies

Alternatively, the anti-Pep356-related antibodies may be monoclonal antibodies. Monoclonal antibodies may be produced by hybridomas, wherein a mouse, hamster, or other appropriate host animal, is immunized with an immunizing agent to elicit lymphocytes that produce or are capable of producing antibodies that will specifically bind to the immunizing agent, e.g. Kohler and Milstein, Nature 256:495 (1975). Alternatively, the lymphocytes may be immunized in vitro. The immunizing agent will typically include the Pep356-related peptides or a fusion protein thereof Generally, spleen cells or lymph node cells are used if non-human mammalian sources are desired, or peripheral blood lymphocytes (“PBLs”) are used if cells of human origin. The lymphocytes are fused with an immortalized cell line using a suitable fusing agent, such as polyethylene glycol, to produce a hybridoma cell, e.g. Goding, MONOCLONAL ANTIBODIES: PRIVCLPLES AND PRACTICE, Academic Press, pp. 59-103 (1986); Liddell and Cryer, A Practical Guide to Monoclonal Antibodies (John Wiley & Sons, New York, 1991); Malik and Lillenoj, Editors, Antibody Techniques (Academic Press, New York, 1994). In general, immortalized cell lines are transformed mammalian cells, for example, myeloma cells of rat, mouse, bovine or human origin. The hybridoma cells are cultured in a suitable culture medium that preferably contains one or more substances that inhibit the growth or survival of unfused, immortalized cells. For exiample, if the parental cells lack the enzyme hypoxanthine guanine phosphoribosyl transferase (HGPRT), the culture medium for the hybridomas typically will include hypoxanthine, aminopterin, and thymidine (HAT), substances which prevent the growth of HGPRT-deficient cells. Preferred immortalized cell lines are those that fuse efficiently, support stable high level production of antibody, and are sensitive to a medium such as HAT medium. More preferred immortalized cell lines are murine or human myeloma lines, which can be obtained, for example, from the American Type Culture Collection (ATCC), Rockville, Md. Human myeloma and mouse-human heteromyeloma cell lines also have been described for the production of human monoclonal antibodies, e.g. Kozbor, J. Immunol. 133:3001 (1984); Brodeur et al., Monoclonaf Antibody Production Techniques and Applications, Marcel Dekker, Inc., New York, pp. 51-63 (1987).

The culture medium (supernatant) in which the hybridoma cells are cultured can be assayed for the presence of monoclonal antibodies directed against a Pep356-related peptide. Preferably, the binding specificity of monoclonal antibodies present in the hybridoma supernatant is determined by irrirnunoprecipitation or by an in vitro binding assay, such-as radio- immunoassay (RIA) or enzyme-linked immunoabsorbent assay (ELISA). Appropriate techniques and assays are known in the art. The binding affinity of the monoclonal antibody can, for example, be determined by the Scatchard analysis of Munson and Pollard, Anal. Biochem. 107:220 (1980). After the desired antibody-producing hybridoma cells are identified, the cells may be cloned by limiting dilution procedures and grown by standard methods [Goding, 1986]. Suitable culture media for this purpose include, for example, Dulbecco's Modified Eagle's Medium and RPMI-1640 medium. Alternatively, the hybridoma cells may be grown in vivo as ascites in a mammal. The monoclonal antibodies secreted by selected clones may be isolated or purified from the culture medium or ascites fluid by immunoglobulin purification procedures routinely used by those of skill in the art such as, for example, protein A-Sepharose, hydroxyl-apatite chromatography, gel electrophoresis, dialysis, or affinity chromatography.

The monoclonal antibodies may also be made by recombinant DNA methods, such as those described in U.S. Pat. No. 4,816,567. DNA encoding the monoclonal antibodies of the invention can be isolated from the Pep356-related peptides-specific hybridoma cells and sequenced, e.g., by using oligonucleotide probes that are capable of binding specifically to genes encoding the heavy and light chains of murine antibodies. Once isolated, the DNA may be inserted into an expression vector, which is then transfected into host cells such as simian COS cells, Chinese hamster ovary (CHO) cells, or myeloma cells that do not otherwise produce immunoglobulin protein, to obtain the synthesis of monoclonal antibodies in the recombinant host cells. The DNA also may be modified, for example by substituting the coding sequence for the mutine heavy and light chain constant domains for homologous human sequences (Morrison et al., Proc. Nat. Acad. Sci. 81:6851-6855 (1984); Neuberger et al., Nature 312:604-608 (1984); Takeda et al., Nature 314:452-454 (1985)), or by covalently joining to the immunoglobulin coding sequence all or part of the coding sequence for a non-immunoglobulin polypeptide. The non-immunoglobulin polypeptide can be substituted for the constant domains of an antibody of the invention, or can be substituted for the variable domains of one antigen-combining site of an antibody of the invention to create a chimeric bivalent antibody. The antibodies may also be monovalent antibodies. Methods for preparing monovalent antibodies are well known in the art. For example, in vitro methods are suitable for preparing monovalent antibodies. Digestion of antibodies to produce fragments thereof, particularly, Fab fragments, can be accomplished using routine techniques known in the art.

Antibodies and antibody fragments characteristic of hybridomas of the invention can also be produced by recombinant means by extracting messenger RNA, constructing a cDNA library, and selecting clones which encode segments of the antibody molecule. The following are exemplary references disclosing recombinant techniques for producing antibodies: Wall et al., Nucleic Acids Research, Vol. 5, pgs. 3113-3128 (1978); Zakut et al., Nucleic Acids Research, Vol. 8, pgs. 3591-3601 (1980); Cabilly et al., Proc. Natl. Acad. Sci., Vol. 81, pgs. 3273-3277 (1984); Boss et al., Nucleic Acids Research, Vol. 12, pgs. 3791-3806 (1984); Amster et al., Nucleic Acids Research, Vol. 8, pgs. 2055-2065 (1980); Moore et al., U.S. Patent 4,642,334; Skerra et al, Science, Vol. 240, pgs. 1038-1041(1988); Huse et al, Science, Vol. 246, pgs. 1275-1281 (1989); and U.S. Pat. Nos. 6,054,297; 5,530,101; 4,816,567; 5,750,105; and 5,648,237, In particular, such techniques can be used to produce interspecific monoclonal antibodies, wherein the binding region of one species is combined with non-binding region of the antibody of another species to reduce inmmunogenicity, e.g. Liu et al., Proc. Natl. Acad. Sci., Vol. 84, pgs. 3439-3443 (1987), and U.S. Pat. Nos. 6,054,297 and 5,530,101. Preferably, recombinantly produced Fab and Fv fragments are expressed in bacterial host systems. Preferably, full-length antibodies are produced by mammalian cell culture techniques. More preferably, full-length antibodies are expressed in Chinese Hampster Ovary (CHO) cells or NSO cells.

Both polyclonal and monoclonal antibodies can be screened by ELISA. As in other solid phase immunoassays, the test is based on the tendency of macromolecules to adsorb nonspecifically to plastic. The irreversibility of this reaction, without loss of immunological activity, allows the formation of antigen-antibody complexes with a simple separation of such complexes from unbound material. To titrate antipeptide serum, peptide conjugated to a carrier different from that used in immunization is adsorbed to the wells of a 96-well microtiter plate. The adsorbed antigen is then allowed to react in the wells with dilutions of anti-peptide serum. Unbound antibody is washed away, and the remaining antigen-antibody complexes are allowed to react with an antibody specific for the IgG of the immunized animal. This second antibody is conjugated to an enzyme such as alkaline phosphatase. A visible colored reaction produced when the enzyme substrate is added indicates which wells have bound antipeptide antibodies., The use of spectrophotometer readings allows better quantification of the amount of peptide-specific antibody bound. High-titer antisera yield a linear titration curve between 10⁻³ and 10⁻⁵ dilutions.

Pep356-related Peptide Carriers

The invention includes immunogens derived from Pep356-related peptides, and immunogens comprising conjugates between carriers and peptides of the invention. The term immunogen as used herein refers to a substance capable of causing an immune response. The termn carrier as used herein refers to any substance which when chemically conjugated to a peptide of the invention permits a host organism immunized with the resulting conjugate to generate antibodies specific for the conjugated peptide. Carriers include red blood cells, bacteriophages, proteins, or synthetic particles such as agarose beads. Preferably, carriers are proteins, such as serum albumin, gamma-globulin, keyhole limpet hemocyanin (KLH), thyroglobulin, ovalbumin, or fibrinogen.

The general technique of linking synthetic peptides to a carrier is described in several references, e.g. Walter and Doolittle, “Antibodies Against Synthetic Peptides,” in Setlow et al., eds., Genetic Engineering, Vol. 5, pgs. 61-91 (Plenum Press, N.Y., 1983); Green et al. Cell, Vol. 28, pgs. 477-487 (1982); Lerner et al., Proc. Natl. Acad. Sci., Vol. 78, pgs. 3403-3407 (1981); Shimizu et al., U.S. Pat. No. 4,474,754; and Ganfield et al., U.S. Pat. No. 4,311,639. Also, techniques employed to link haptens to carriers are essentially the same as the above-referenced techniques, e.g. chapter 20 in Tijssen, Practice and Theory of Enzyme Inmunoassays (Elsevier, New York, 1985). The four most commonly used schemes for attaching a peptide to a carrier are (1) glutaraldehyde for amino coupling, e.g. as disclosed by Kagan and Glick, in Jaffe and Behrmnan, eds. Methods of Hormone Radioinmmunoassay, pgs. 328-329 (Academic Press, N.Y., 1979), and Walter et al. Proc. Natl. Acad. Sci., Vol. 77, pgs. 5197-5200 (1980); (2) water-soluble carbodiimides for carboxyl to amino coupling, e.g. as disclosed by Hoare et al., J. Biol. Chem., Vol. 242, pgs. 2447-2453 (1967); (3) bis-diazobenzidine (BDB) for tyrosine to tyrosine sidechain coupling, e.g. as disclosed by Bassiri et al., pgs. 46-47, in Jaffe and Behrman, eds. (cited above), and Walter et al. (cited above); and (4) maleimidobenzoyl-N-hydroxysuccinimide ester (MBS) for coupling cysteine (or other sulfhydryls) to amino groups, e.g. as disclosed by Kitagawa et al., J. Biochem. (Tokyo), Vol. 79, pgs. 233-239 (1976), and Lemer et al. (cited above). A general rule for selecting an appropriate method for coupling a given peptide to a protein carrier can be stated as follows: the group involved in attachment should occur only once in the sequence, preferably at the appropriate end of the segment. For example, BDB should not be used if a tyrosine residue occurs in the main part of a sequence chosen for its potentially antigenic character. Similarly, centrally located lysines rule out the glutaraldehyde method, and the occurrences of aspartic and glutamic acids frequently exclude the carbodiimide approach. On the other hand, suitable residues can be positioned at either end of chosen sequence segment as attachment sites, whether or not they occur in the “native” protein sequence. Internal segments, unlike the amino and carboxy termini, avil differ significantly at the “unattached end” from the same sequence as it is found in the native protein where the polypeptide backbone is continuous. The problem can be remedied, to a degree, by acetylating the α-amino group and then attaching the peptide by way of its carboxy terminus. The coupling efficiency to the carrier protein is conveniently measured by using a radioactively labeled peptide, prepared either by using a radioactive amino acid for one step of the synthesis or by labeling the completed peptide by the iodination of a tyrosine residue. The presence of tyrosine in the peptide also allows one to set up a sensitive radioirunune assay, if desirable. Therefore, tyrosine can be introduced as a terminal residue if it is not part of the peptide sequence defined by the native polypeptide.

Peptides can be linked to KLH through cysteines by MBS as disclosed by Liu et al., Biochemistry, Vol. 18, pgs. 690-697 (1979). The peptides are dissolved in phosphate-buffered saline (pH 7.5), 0.1 M sodium borate buffer (pH 9.0) or 1.0 M sodium acetate buffer (pH 4.0). The pH for the dissolution of the peptide is chosen to optimize peptide solubility. The content of free cysteine for soluble peptides,is determined by Ellman's method, Ellman, Arch. Biochem. Biophys., Vol. 82, pg. 7077 (1959). For each peptide, 4 mg KLH in 0.25 ml of 10 mM sodium phosphate buffer (pH 7.2) is reacted with 0.7 mg MBS (dissolved in dimethγ1 fonnarnide) and stirred for 30 min at room temperature. The MBS is added dropwise to ensure that the local concentration of formamide is not too high, as KLH is insoluble in >30% formamide. The reaction product, KLH-MBS, is then passed through Sephadex G-25 equilibrated with 50 mM sodium phosphate buffer (pH 6.0) to remove free MBS, KLH recovery from peak fractions of the column eluate (monitored by OD280) is estimated to be approximately 80%. KLH-MBS is then reacted with 5 mg peptide dissolved in 1 ml of the chosen buffer. The pH is adjusted to 7-7.5 and the reaction is stirred for 3 hr at room temperature. Coupling efficiency is monitored with radioactive peptide by dialysis of a sample of the conjugate against phosphate-buffered saline, and may range from 8% to 60%. Once the peptide-carrier conjugate is available, polyclonal or monoclonal antibodies are produced by standard techniques, e.g. as disclosed by Campbell, Monoclonal Antibody Technology (Elsevier, New York, 1984); Hurrell, ed. Monoclonal Hybridoma Antibodies: Techniques and Applications (CRC Press, Boca Raton, Fla., 1982); Schreier et al. Hybridoma Techniques (Cold Spring Harbor Laboratory, New York, 1980); U.S. Pat. No. 4,562,003.

Humanized Antibodies

The anti-Pep356-related antibodies of the invention may further comprise humanized antibodies or human antibodies. The term “humanized antibody” refers to humanized forms of non-human (e.g., murine) antibodies that are chimeric antibodies, immunoglobulin chains or fragments thereof (such as Fv, Fab, Fab′, F(ab′), or other antigen-binding partial sequences of antibodies) which contain some portion of the sequence derived from non-human antibody. Humanized antibodies include human imrnunoglobulins in which residues from a complementary determining region (CDR) of the human immunoglobulin are replaced by residues from a CDR of a non-human species such as mouse, rat or rabbit having the desired binding specificity, affinity and capacity. In general, the humanized antibody will comprise substantially all of at least one, and generally two, variable domains, in which all or substantially all of the CDR regions correspond to those of a non-human inmuunoglobulin and all or substantially all of the FR regions are those of a human immunoglobulin consensus sequence. The humanized antibody optimally also will comprise at least a portion of an immunoglobulin constant region (Fc), typically that of a human inununoglobulin (Jones et al., Nature 321:522-525 (1986) and Presta, Curr. Op. Struct. Biol 2:593-596 (1992)). Methods for humanizing non-human antibodies are well known in the art. Generally, a humanized antibody has one or more amino acids introduced into it from a source which is non-human in order to more closely resemble a human antibody, while still retaining the original binding activity of the antibody. Methods for humanization of antibodies are further detailed in Jones et al., Nature 321:522-525 (1986); Riechmann et al., Nature 332:323-327 (1988); and Verhoeyen et al., Science 239:1534-1536 (1988). Such “humanized” antibodies are chimeric antibodies in that substantially less than an intact human variable domain has been substituted by the corresponding sequence from a non-human species.

Heteroconjugate Antibodies

Heteroconjugate antibodies which comprise two covalently joined antibodies, are also within the scope of the present invention. Heteroconjugate antibodies may be prepared in vitro using knowvn methods in synthetic protein chemistry, including those involving crosslinking agents. For example, immunotoxins may be prepared using a disulfide exchange reaction or by forming a thioether bond.

Bispecific Antibodies

Bispecific antibodies have binding specificities for at least two different antigens. Such antibodies are monoclonal, and preferably human or humanized. One of the binding specificities of a bispecific antibody of the present invention is for a Pep356-related peptides, and the other one is preferably for a cell-surface protein or receptor or receptor subunit. Methods for making bispecific antibodies are known in the art, and in general, the recombinant production of bispecific antibodies is based on the co-expression of tnvo immunoglobulin heavy-chain/light-chain pairs in hybridoma cells, where the two heavy chains have different specificities, e.g. Milstein and Cuello, Nature 305:537-539 (1983). Given that the random assortment of immunoglobulin heavy and light chains results in production of potentially ten different antibody molecules by the hybridomas, purification of the correct molecule usually requires some sort of affinity purification, e.g. affinity chromatography.

Uses of Anti-Pep356-related Antibodies

Antibodies specific for Pep356-related peptides may be used as functional antagonists. Preferably, the antagonists of the invention comprise fragments or binding compositions specific for Pep356-related peptides. Preferably, antagonists of the invention are derived from monoclonal antibodies specific for Pep356-related peptides. Monoclonal antibodies capable of blocking, or neutralizing, Pep356-related peptides are selected by their ability to inhibit Pep356-related peptides-induced effects.

The use and generation of fragments of antibodies is also well known, e.g. Fab fragments: Tijssen, Practice and Theory of Enzyme Immunoassays (Elsevier, Amsterdam, 1985); and Fv fragments: Hochman et al. Biochemistry, Vol. 12, pgs. 1130-1135 (1973), Sharon et al., Biochemistry, Vol. 15, pgs. 1591-1594 (1976) and Ehrlich et al., U.S. Pat. No. 4,355,023; and antibody half molecules: Auditore-Hargreaves, U.S. Pat. No. 4,470,925.

Preferably, monoclonal antibodies, Fv fragments, Fab fragments, or other binding compositions derived from monoclonal antibodies of the invention have a high affinity to Pep356-related peptides. The affinity of monoclonal antibodies and related molecules to Pep356-related peptides may be measured by conventional techniques including plasmon resonance, ELISA, equilibrium dialysis, and the like. Affinity measurement by plasmon resonance techniques may be carried out, for example, using a BIAcore 2000 instrument (Biacore AB, Uppsala, Sweden) in accordance with the manufacturer's recommended protocol. Preferably, is measured by ELISA, for example, as described in U.S. Pat. No. 6,235,883, or like reference. Preferably, the dissociation constant between Pep356-related peptides and monoclonal antibodies of the invention is less than 10⁻⁵ molar. More preferably, such dissociation constant is less than 10⁻⁸ molar; still more preferably, such dissociation constant is less than 10⁻⁹ molar; and most preferably, such dissociation constant is in the range of 10⁻⁹ to 10⁻¹¹ molar.

In addition, the antibodies of the present invention are useful for detecting Pep356-related peptides. Such detection methods are advantageously applied to diagnosis and prognosis of, e.g., aberrant cell growth conditions. The antibodies of the inverition may be used in most assays involving antigen-antibody reactions. The assays may be homogeneous or heterogeneous. In a homogeneous assay approach, the sample can be a biological sample or fluid such as serum, urine, whole blood, lymphatic fluid, plasma, saliva, cells, tissue, and material secreted by cells or tissues cultured in vitro. The sample can be pretreated if necessary to remove unwanted materials. The immunological reaction usually involves the specific antibody, labeled analyte, and the sample suspected of containing the analyte. The analyte can be directly labeled with the label or indirectly labeled with a means for incorporating the label such as conjugation of the analyte to biotin and having labeled avidin or anti-biotin. The signal from the label is modified, directly or indirectly, upon the binding of the antibody of the labeled analyte. Both the immunological reaction and detection of the extent thereof are carried out in a homogeneous solution. In a heterogeneous assay approach, the reagents are usually the sample, the specific antibody, and means for producing a detectable signal. The specimen is generally placed on a support, such as a plate or a slide, and contacted with the antibody in a liquid phase. The support is then separated from the liquid phase and either the support phase or the liquid phase is examined for a detectable signal employing means for producing such signal or signal producing system. The signal is related to the presence of the analyte in the sample. Means for producing a detectable signal includes the use of radioactive labels, fluorescers, enzymes, and so forth. Exemplary of heterogeneous immunoassays are the radioimmunoassay, immunofluorescence methods, enzyme-linked immunoassays, and the like.

One embodiment of an assay employing an antibody of the present invention involves the use of a surface to which the monoclonal antibody of the invention is attached. The underlying structure of the surface may take different forms, have different compositions and may be a mixture of compositions or laminates. The surface may assume a variety of shapes and forms and may have varied dimensions, depending on the manner of use and measurement. Illustrative surfaces may be pads, beads, discs, or strips which may be flat, concave or convex. Thickness is not critical, generally being from about 0.1 to 2 mm thick and of any convenient diameter or other dimensions. The surface typically will be supported on a rod, tube, capillary, fiber, strip, disc, plate, cuvette and the like. The surface will typically be porous and polyfunctional or capable of being polyfunctionalized so as to permit covalent binding of the monoclonal antibody of the invention as well as to permit bonding of other compounds which form a part of a means for producing a detectable signal. A wide variety of organic and inorganic polymers, both natural and synthetic, and combinations thereof, may be employed as the material for the solid surface. Illustrative polymers include polyethylene, polypropylene, poly(4-methylbutene), polystyrene, polymethracrylate, poly(ethylene terephthalate), rayon, nylon, poly(vinyl butyrate), silicones, polyformaldehyde, cellulose, cellulose acetate, nitrocellulose, latex, etc. Other usefuil materials include paper, glasses, ceramics, metals, metaloids, semiconductor materials, cermets, silicates, substrates that form gels, gelatins, lipopolysaccharides, silicates, agarose, polyacrylamides, polymers which form several aqueous phases such as dextrans, polyalkylene glycols (alkylene of 2 to 3 carbon atoms) or surfactants such as phospholipids. The binding of the monoclonal antibody of the invention to the surface may be accomplished by well known techniques, for example, “Immobilized Enzymes,” Ichiro Chibata, Press, New York (1978) and Cuatrecasas, J. Bio. Chem., 245: 3059 (1970). In carrying out the assay in accordance with this of the invention the sample is mixed with aqueous medium and the medium is contacted with the surface having a monoclonal antibody of the invention bound thereto. Members of a signal producing system and any ancillary materials may also be included in the aqueous medium, either concurrently or added subsequently so as to provide a detectable signal associated with the surface. The means for producing the detectable signal can involve the incorporation in the of a labeled analyte or it may involve the use of a second monoclonal antibody having a label conjugated thereto. Separation and washing steps will be carried out as needed. The signal detected is related to the presence of Pep356-related peptides in the sample. It is within the scope of the present invention to include a calibration as the measurement surface on the same support. A particular embodiment of an assay in accordance with the present invention, by way of illustration and not limitation, involves the use of a support such as a slide or a well of a petri dish. The technique involves the sample to be analyzed on the support with an appropriate fixing material such as acetone and incubating the sample on the slide with a monoclonal antibody of the invention. After washing with an appropriate buffer such as, for example, phosphate buffered saline, the support is contacted with a labeled specific binding partner for the analyte in the sample. After incubation as desired, the slide is washed a second time with an aqueous buffer and the determination is made of the binding of the labeled monoclonal antibody to the analyte. If the label is fluorescent, the.slide may be covered with a fluorescent antibody mounting fluid on a cover slip and then examined with a fluorescent microscope to determine the extent of binding. On the other hand, the label can be an enzyme conjugated to the monoclonal antibody of the invention and the extent of binding can be determined by examining the slide for the presence of enzyme activity, which may be indicated by the formation of a precipitate, a color, or the like. A particular example of an assay utilizing the present antibodies is a double determinant ELISA assay. A support such as, e.g., a glass or vinyl plate, is coated with antibody specific for Pep356-related peptides by conventional techniques. The support is contacted with the sample suspected of containing Pep356-related peptides, usually in aqueous medium. After an incubation period from 30 seconds to 12 hours, the support is separated from the medium, washed to remove unbound Pep356-related peptides with, for example, water or an aqueous buffered rnedium, and contacted with an antibody specific for Pep356-related peptides, again usually in an aqueous medium. The antibody is labeled with an enzyme directly or indirectly such as, e.g., horseradish peroxidase or alkaline phosphatase. After incubation, the support is separated from the medium, and washed as above. The enzyme activity of the support or the aqueous medium is determined. This enzyme activity is related to the amount of Pep356-related peptides in the sample.

The invention also includes kits, e.g., diagnostic assay kits, for carrying out the methods disclosed above. In one embodiment, the kit comprises in packaged combination (a) a monoclonal antibody more specifically defined above and (b) a conjugate of a specific binding partner for the above monoclonal antibody and a label capable of producing a detectable signal. The reagents may also include ancillary agents such as buffering agents and protein stabilizing agents, e.g., polysaccharides and the like. The kit may further include, where necessary, other members of the signal producing system of which system the label is a member, agents for reducing background interference in a test, control reagents, apparatus for conducting a test, and the like. In another embodiment, the diagnostic kit comprises a conjugate of monoclonal antibody of the invention and a label capable of producing a detectable signal. Ancillary agents as mentioned above may also be present.

An anti-Pep356-related antibody (e.g., monoclonal antibody) can be used to isolate Pep356-related peptide by standard techniques, such as affinity chromatography or inununoprecipitation. For example, an anti-Pep356-related antibody can facilitate the purification of endogenous or recombinantly expressed Pep356-related peptides from cells. Moreover, an anti-Pep356-related antibody can be used to detect Pep356-related peptide (e.g., in a cellular lysate or cell supernatant) in order to evaluate the abundance and pattern of expression of the Pep 356-relaied peptide. Anti-Pep356-related antibodies can be used diagnostically to monitor protein levels in tissue as part of a clinical testing procedure, e.g., to, for example, determine the efficacy of a given treatment regimen. Detection is facilitated by linking the antibody to any label group.

Protein Arrays

Detection, purification, and screening of the polypeptides of the invention may be accomplished using retentate chromatography (preferably, protein arrays or chips), as described by U.S. Pat. No. 6,225,027 and U.S. Patent Application 20010014461. Briefly, retentate chromatography describes methods in which polypeptides (and/ or other sample components) are retained on an adsorbent (e.g., array or chip) and subsequently detected. Such methods involve (1) selectively adsorbing polypeptides from a sample to a substrate under a plurality of different adsorbent/eluant combinations (“selectivity conditions”) and (2) detecting the retention of adsorbed polypeptides by desorption spectrometry (e.g., by mass spectrometry). In conventional chromatographic methods, polypeptides are eluted off of the adsorbent prior to detection. The coupling of adsorption chromatography with detection by desorption spectrometry provides extraordinary sensitivity, the ability to rapidly analyze retained components with a variety of different selectivity conditions, and parallel processing of components adsorbed to different sites (i.e., “affinity sites” or “spots”) on the array under different elution conditions.

These methods are useful for: combinatorial, biochemical separation and purification of the Pep356-related peptides; study of differential gene expression; detection of differences in protein levels (e.g., for diagnosis), and detection of molecular recognition events, (e.g., for screening and drug discovery). Thus, this invention provides a molecular discovery and diagnostic device that is characterized by the inclusion of both parallel and multiplex polypeptide processing capabilities. Polypeptides of the invention and Pep356-related peptide-binding substances are preferably attached to a label group, and thus directly detected, enabling simultaneous transmission of two or more signals from the same “circuit” (i.e., addressable “chip” location) during a single unit operation.

Detection of Pep356-related Peptides By Mass Spectrometry

In accordance with the present invention, any instrument, method, process, etc. can be utilized to determine the identity and abundance of proteins in a sample. A preferred method of obtaining identity is by mass spectrometry, where protein molecules in a sample are ionized and then the resultant mass and charge of the protein ions are detected and determined.

To use mass spectrometry to analyze proteins, it is preferred that the protein be converted to a gas-ion phase. Various methods of protein ionization are useful, including, e.g., fast ion bombardment (FAB), plasma desorption, laser desorption, thermal desorption, preferably, electrospray ionization (ESI) and matrix-assisted laser desorption/ionization (MALDI). Many different mass analyzers are available for peptide and protein analysis, including, but not limited to, Time-of-Flight (TOF), ion trap (ITMS), Fourier transform ion cyclotron (FTMS), quadrupole ion trap, and sector (electric and/or magnetic) spectrometers. See, e.g., U.S. Pat. No. 5,572,025 for an ion-trap MS. Mass analyzers can be used alone, or in combination with other mass analyzers in tandem mass spectrometers. In the latter case, a first mass analyzer can be use to separate the protein ions (precursor ion) from each other and determine the molecular weights of the various protein constituents in the sample. A second mass analyzer can be used to analyzc each separated constituents, e.g., by fragmenting the precursor ions into product ions by using, e.g. an inert gas. Any desired combination of mass analyzers can be used, including, e.g., triple quadrupoles, tandem time-of-flights, ion traps, and/or combinations thereof.

Different kinds of detectors can be used to detect the protein ions. For example, destructive detectors can be utilized, such as ion electron multipliers or cryogenic detectors (e.g., U.S. Pat. No. 5,640,010). Additionally, non-destructive detectors can be used, such as ion traps which are used as ion current pick-up devices in quadrupole ion trap mass analyzers or FTMS.

For MALDI-TOF, a number of sample preparation methods can be utilized including, dried droplet (Karasand Hillenkamp, Anal. Chem., 60:2299-2301, 1988), vacuum-drying (Winberger et al., In Proceedings of the 41st ASMS Conference on Mass Spectrometry and Allied Topics, San Francisco, May 31-Jun. 4, 1993, pp. 775a-b), crush crystals (Xiang et al., Rapid Comm. Mass Spectrom., 8:199-204,1994), slow crystal growing (Xiang et al., Org. Mass Spectrom, 28:1424-1429, 1993); active film (Mock et al., Rapid Comm. Mass Spectrom.,6:233-238, 1992; Bai et al., Anal. Chem., 66:3423-3430, 1994), pneumatic spray (Kochling et al., Proceedings of the 43rd ASMS Conference on Mass Spectrometry and Allied Topics; Atlanta, Ga., May 21-26, 1995, p 1225); electrospray (Hensel et al., Proceedings of the 43rd ASMS Conference on Mass Spectrometry and Allied Topics; Atlanta, Ga., May 21 -26, 1995, p947); fast solvent evaporation (Vorm et al., Anal. Chem., 66:3281-3287, 1994); sandwich (Li et al., J. Am. Chem. Soc., 11 8: 11662-11663,1996); and two-layer methods (Dal et al., Anal. Chem., 71:1087-1091, 1999). See also, e.g., Liang et al., Rapid Commun. Mass Spectrom., 10: 1219-1226, 1996; van Adrichemet al., Anal. Chem., 70:923-930, 1998.

For MALDI analysis, samples are prepared as solid-state co-crystals or thin films by mixing them with an energy absorbing compound or colloid (the matrix) in the liquid phase, and ultimately drying the solution to the solid state upon the surface of an inert probe. In some cases an energy absorbing molecule (EAM) is an integral component of the sample presenting surface. Regardless of EAM application strategy, the probe contents are allowed to dry to the solid state prior to introduction into the laser desorption/ionization time-of-flight mass spectrometer (LDIMS).

Ion detection in TOF mass spectrometry is typically achieved with the use of electro-emissive detectors such as electron multipliers (EMP) or microchannel plates (MCP). Both of these devices function by converting primary incident charged particles into a cascade of secondary, tertiary, quaternary, etc. electrons. The probability of secondary electrons being generated by the impact of a single incident charged particle can be taken to be the ion-to-electron conversion efficiency of this charged particle (or more simply, the conversion efficiency). The total electron yield for cascading events when compared to the total number of incident charged particles is typically described as the detector gain. Because generally the overall response time of MCPs is far superior to that of EMPs, MCPs are the preferred electro-emissive detector for enhancing mass/charge resolving power. However, EMPs function well for detecting ion populations of disbursed kinetic energies, where rapid response time and broad frequency bandwidth are not necessary.

In a preferred aspect, for the analysis of digested proteins, a liquid-chromatography tandem mass spectrometer (LC-TMS) is used. This system provides an additional stage of sample separation via use of a liquid chromatograph followed by tandem mass spectrometry.

The methods described herein of separating and fractionating proteins provide individual proteins or fractions containing small numbers of distinct proteins. These proteins can be identified by mass spectral determination of the molecular masses of the protein and peptides resulting from the fragmentation thereof. Making use of available information in protein sequence databases, a comparison can be made between proteolytic peptide mass patterns generated in silico, and experimentally observed peptide masses. A “hit-list” can be compiled, ranking candidate proteins in the database, based on (among other criteria) the number of matches between the theoretical and experimental proteolytic fragments: Methods of peptide mapping and sequencing using MS are described in WO 95/252819, U.S. Pat. No. 5,539,897, U.S. Pat. No. 5,869,240, U.S. Pat. No. 5,572,259, and U.S. Pat. No. 5,696,376. See, also, Yates, J. Mass Spec., 33:1 (1998).

Data collected from a mass spectrometer typically comprises the intensity and mass to charge ratio for each detected event. Spectral data can be recorded in any suitable form, including, e.g., in graphical, numerical, or electronic formats, either in digital or analog form. Spectra are preferably recorded in a storage medium, including, e.g., magnetic, such as floppy disk, tape, or hard disk; optical, such as CD-ROM or laser-disc, or, ROM-CHIPS.

The mass spectrum of a given sample typically provides information on protein intensity, mass to charge ratio, and molecular weight. In preferred embodiments of the invention, the molecular weights of proteins in the sample are used as a matching criterion to query a database. The molecular weights are calculated conventionally, e.g., by subtracting the mass of the ionizing proton for singly-charged protonated molecular ions, by multiplying the measured mass/charge ratio by the number of charges for multiply-charged ions and subtracting the number of ionizing protons.

Various databases are useful in accordance with the present invention. Useful databases include, databases containing genomic sequences, expressed gene sequences, and/or expressed protein sequences. Preferred databases contain nucleotide sequence-derived molecular masses of proteins present in a known organism, organ, tissue, or cell-type. There are a number of algorithms to identiy open reading frames (ORF) and convert nucleotide sequences into protein sequence and molecular weight information. Several publicly accessible databases are available, including, the SwissPROT/TrEMBL database (http://www.expasy.ch).

Typically, a mass spectrometer is equipped with commercial software that identifies peaks above a certain threshold level, calculates mass, charge, and intensity of detected ions. Correlating molecular weight with a given output peak can be accomplished directly from the spectral data, i.e., where the charge on an ion is one and the molecular weight is therefore equal to the numerator value minus the mass of the ionizing proton. However, protein ions can be complexed with various counter-ions and adducts, such as N, C, and K′. In such a case, it would be expected that a given protein ion wvould exhibit multiple peaks, such as a triplet, representing different ionic states (or species) of the same protein. Thus, it may be necessary to analyze and process spectral data to determine families of peaks arising from the same protein. This analysis can be carried out conventionally, e.g., as described by Mann et al., anal. Chem., 61:1702-1708 (1989).

In matching a molecular mass calculated from a mass spectrometer to a molecular mass predicted from a database, such as a genomic or expressed gene database, post-translation processing may have to be considered. There are various processing events which modify protein structure, including, proteolytic processing, removal of N-terminal methionine, acetylation, methylation, glycosylation, phosphorylation, etc.

A database can be queried for a range of proteins matching the molecular mass of the unknown. The range window can be determined by the accuracy of the instrument, the method by which the sample was prepared, etc. Based on the number of hits (where a hit is match) in the spectrum, the unknown protein or peptide is identified or classified.

Methods of identifying one or more Pep356-related peptide by mass spectrometry are useful for detection of one or more Pep356-related peptide present in human plasma. Exemplary techniques are described in U.S. Patent Applications 02/0060290, 02/0137106, 02/0138208, 02/0142343, 02/0155509.

Diagnostic and Prognostic Uses

The nucleic acid molecules, proteins, protein homologues, and antibodies described herein can be used in one or more of the following methods: diagnostic assays, prognostic assays, monitoring clinical trials, drug screening and pharmacogenetics as further described herein.

The invention provides diagnostic and prognostic assays for detecting Pep356-related peptide nucleic acids and proteins, as further described. Also provided are diagnostic and prognostic assays for detecting interactions between Pep356-related peptides and target molecules, particularly natural agonists and antagonists.

The present invention provides methods for identifying polypeptides that are differentially expressed between two or more samples. “Differential expression” refers to differences in the quantity or quality of a polypeptide between samples. Such differences could result at any stage of protein expression from transcription through post-translational modification. For example, using protein array methods, two samples are bound to affinity spots on different sets of adsorbents (e.g., chips) and recognition maps are compared to identify polypeptides that are differentially retained by the two sets of adsorbents. Differential retention includes quantitative retention as well as qualitative differences in the polypeptide. For example, differences in post-translational modification of a protein can result in differences in recognition maps detectable as differences in binding characteristics (e.g., glycosylated proteins bind differently to lectin adsorbents) or differences in mass (e.g., post-translational cleavage products). In certain embodiments, an adsorbent can have an array of affinity spots selected for a combination of markers diagnostic for a disease or syndrome.

Differences in polypeptide levels between samples (e.g., differentially expressed Pep356-related peptides in plasma samples) can be identified by exposing the samples to a variety of conditions for analysis by desorption spectrometry (e.g., mass spectrometry). Unknown proteins can be identified by detecting physicochernical characteristics (e.g., molecular mass), and this information can be used to search databases for proteins having similar profiles.

Preferred methods of detecting a Pep356-related peptide utilize mass spectrometry techniques. Such methods provide information about the size and character of the particular Pep356-related peptide isoform that is present in a sample, e.g., a biological sample submitted for diagnosis or prognosis. Mass spectrometry techniques are detailed in the section titled “Detection of Pep356-related peptides by mass spectrometry”. The invention provides a method of detecting a Pep356-related peptide in a biological sample comprising the steps of: fractionating a biological sample (e.g., plasma, serum, lymph, cerebrospinal fluid, cell lysate of a particular tissue) by at least one chromatographic step; subjecting a fraction to mass spectrometry; comparing the characteristics of peptide species observed in mass spectrometry with known characteristics of Pep356-related peptides.

The isolated nucleic acid molecules of the invention can be used, for example, to detect Pep356-related mRNA (e.g., in a biological sample) or a genetic alteration in a Pep356-encoding gene, and to modulate a Pep356-related peptide activity, as described further below. The Pep356-related peptide can be used to treat disorders characterized by insufficient production of a Pep356-related peptide or by excessive production of a Pep356-related peptide target molecule. In addition, the Pep356-related peptides can be used to screen for naturally occurring target molecules, to screen for drugs or compounds which modulate, preferably activate Pep356-related peptide activity, as well as to treat disorders characterized by insufficient production of Pep356-related peptide or production of Pep356-related peptide forms which have decreased or aberrant activity compared to wild type. Moreover, the anti-Pep356-related peptide antibodies of the invention can be used to detect and isolate Pep356-related peptide, regulate the bioavailability of Pep356-related peptide, and modulate Pep356-related peptide activity.

Accordingly one embodiment of the present invention involves a method of use (e.g., a diagnostic or prognostic assay) wherein a molecule of the present invention (e.g., a Pep356-related peptide, nucleic acid, antibody, or modulator) is used, for example, to diagnose, prognose and/or treat a disorder in which any of the aforementioned Pep356-related peptide activities is indicated. In another embodiment, the present invention involves a method of use wherein a molecule of the present invention is used, for example, for the diagnosis, prognosis, and/or treatment of subjects, preferably a human subject, in which any of the aforementioned activities is pathologically perturbed. In a preferred embodiment, the methods of use involve administering to a subject, preferably a human subject, a molecule of the present invention for the diagnosis, prognosis, and/or therapeutic treatment. In another embodiment, the methods of use involve administering to a human subject a molecule of the present invention.

For example, the invention encompasses a method of determining whether a Pep356-related peptide is expressed within a biological sample comprising: a) contacting said biological sample with: i) a polynucleotide that hybridizes under stringent conditions to a Pep356-related nucleic acid; or ii) a detectable polypeptide (e.g. antibody) that selectively binds to a Pep356-related peptide; and b) detecting the presence or absence of hybridization between said polynucleotide and an RNA species within said sample, or the presence or absence of binding of said detectable polypeptide to a polypeptide within said sample. Detection of said hybridization or of said binding indicates that said Pep356-related peptide is expressed within said sample. Preferably, the polynucleotide is a primer, and wherein said hybridization is detected by detecting the presence of an amplification product comprising said primer sequence, or the detectable polypeptide is an antibody.

In certain embodiments, detection involves the use of a probe/primer in a polymerase chain reaction (PCR) (see, e.g., U.S. Pat. Nos. 4,683,195 and 4,683,202), such as anchor PCR or RACE PCR, or, alternatively, in a ligation chain reaction (LCR) (see, e.g., Landegren et al. (1988) Science 241:1077-1080; and Nakazawa et al. (1994) PNAS 91:360-364), the latter of which can be particularly useful for detecting point mutations in the Pep356-related peptide-encoding-gene (see Abravaya et al. (1995) Nucleic Acids Res. 23:675-682).

Also envisioned is a method of determining whether a mammal, preferably human, has an elevated or reduced level of expression of a Pep356-related peptide, comprising: a) providing a biological sample from said mammal; and b) comparing the amount of a Pep356-related peptide or of a Pep356-related RNA species within said biological sample with a level detected in or expected from a control sample. An increased amount of said Pep356-related peptide or said RNA species within said biological sample compared to said level detected in or expected from said control sample indicates that said mammal has an elevated level of Pep356-related peptide expression, and a decreased amount of said Pep356-related peptide or said RNA species within said biological sample compared to said level detected in or expected from said control sample indicates tnat said manunal has a reduced level of expression of a Pep356-related peptide.

The present invention also pertains to the field of predictive medicine in which diagnostic assays, prognostic assays, and monitoring clinical trials are used for prognostic purposes to thereby treat an individual prophylactically. Accordingly, one aspect of the present invention relates to diagnostic assays for determining Pep356-related peptide and/or nucleic acid expression as well as Pep356-related peptide activity, in the context of a biological sample (e.g., blood, plasma, serum, cells, tissue) to thereby determine whether an individual is afflicted with a disease or disorder, or is at risk of developing a disorder, associated with aberrant Pep356-related peptide expression or activity. The invention also provides for prognostic (or predictive) assays for determining whether an individual is at risk of developing a disorder associated with a Pep356-related peptide, nucleic acid expression or activity. For example, mutations in a Pep356-related peptide -encoding gene can be assayed in a biological sample. Such assays can be used for prognostic or predictive purpose to thereby prophylactically treat an individual prior to the onset of a disorder characterized by or associated with Pep356-related peptide expression or activity.

The term “biological sample” is intended to include tissues, cells and biological fluids isolated from an individual, as well as tissues, cells and fluids present within an individual. That is, the detection methods of the invention can be used to detect a Pep356-related peptide mRNA, protein, or genomic DNA in a biological sample in vitro as well as in vivo. Preferred biological samples are biological fluids such as lymph, cerebrospinal fluid, blood, and especially blood plasma and serum. For example, in vitro techniques for detection of a Pep356-related peptide mRNA include Northern hybridizations and in situ hybridizations. In vitro techniques for detection of a Pep356-related peptide include mass spectrometry, enzyme linked immunosorbent assays (ELISAs), Western blots, immunoprecipitations and immunofluorescence. In vitro techniques for detection of a Pep356-related peptide-encoding genomic DNA include Southern hybridizations. Furthermore, in vivo techniques for detection of a Pep356-related peptide include introducing into an individual a labeled anti-Pep356-related peptide antibody.

In preferred embodiments, the subject methods can be characterized by generally comprising detecting, in a tissue sample of theindividual (e.g. a human patient), the presence or absence of a genetic lesion characterized by at least one of (i) a mutation of a gene encoding one of the subject Pep356-related peptide or (ii) the mis-expression of a Pep356 gene. To illustrate, such genetic lesions can be detected by ascertaining the existence of at least one of (i) a deletion of one or more nucleotides from the Pep356 gene, (ii) an addition of one or more nucleotides to the gene, (iii) a substitution of one or more nucleotides of the gene, (iv) a gross chromosomal rearrangement or amplification of the gene, (v) a gross alteration in the level of a messenger RNA transcript of the gene, (vi) aberrant modification of the gene, such as of the methylation pattern of the genomic DNA, (vii) the presence of a non-wild type splicing pattern of a messenger RNA transcript of the gene, and (viii) reduced level of expression, indicating lesion in regulatory element or reduced stability of a Pep356-related transcript.

In yet another exemplary embodiment, aberrant methylation patterns of a Pep356-related peptide nucleic acid can be detected by digesting genomic DNA from a patient sample with one or more restriction endonucleases that are sensitive to methylation and for which recognition sites exist in the Pep356 gene (including in the flanking and intronic sequences). See, for example, Buiting et al. (1994) Human Mol Genet 3:893-895. Digested,DNA is separated by gel electrophoresis, and hybridized with probes derived from, for example, genomic or cDNA sequences. The methylation status of the Pep356 gene can be deterrnined by comparison of the restriction pattern generated from the sample DNA with that for a standard of known methylation.

In yet another embodiment, a diagnostic assay is provided which detects the ability of a Pep356-related peptide to bind to a cell surface ot extracellular protein. For instance, it will be desirable to detect Pep356-related peptide mutants which, while expressed at appreciable levels in the cell, are defective at binding a Pep356-related peptide target protein (having either diminished or enhanced binding affinity for the target). Such mutants may arise, for example, from mutations, e.g., point mutants, which may be impractical to detect by the diagnostic DNA sequencing techniques or by the imnmunoassays described above. The present invention accordingly further contemplates diagnostic screening assays which generally comprise cloning one or more Pep356 gene from the sample tissue, and expressing the cloned genes under conditions which permit detection of an interaction between that recombinant gene product and a target protein. As will be apparent from the description of the various assays set forth herein under the Section entitled “Drug screening assays”, a wide variety of techniques can be used to determine the ability of a Pep356-related peptide to bind to other components. These techniques can be used to detect mutations in a Pep356 gene which give rise to mutant proteins with a higher or lower binding affinity for a Pep356 target protein relative to the wild-type Pep356-related peptide. Conversely, by switching which of the Pep356 target protein and Pep356-related peptideAis the “bait” and which is derived from the patient sample, the subject assay can also be used to detect target protein mutants which have a higher or lower binding affinity for a Pep356-related peptide relative to a wild type form of that target protein.

In an exemplary embodiment, a target protein can be provided as an immobilized protein (a “target”), such as by use of GST fusion proteins and glutathione treated microtitre plates as described herein.

In another embodiment, the methods further involve obtaining a control biological sample from a control subject, contacting the control sample with a compound or agent capable of detecting a Pep356-related peptide, mRNA, or genomic DNA, such that the presence of a Pep356-related peptide, mRNA or gencmic DNA is detected in the biological sample, and comparing the presence of a Pep356-related peptide, mRNA or genomic DNA in the control sample with the presence of a Pep356-related peptide, mRNA or genomic DNA in the test sample. The invention also encompasses kits for detecting the presence of a Pep356-related peptide, rmRNA or genomic DNA in a biological sample. For example, the kit comprises: a labeled compound or agent capable of detecting a Pep356-related peptide, mRNA or genomic DNA in a biological sample; means for determining the amount of a Pep356-related peptide in the sample; and means for comparing the amount of Pep356-related peptide in the sample with a standard. The compound or agent can be packaged in a suitable container. The kit can further comprise instructions for using the kit to detect Pep356-related peptide or nucleic acid.

Drug Screening Assays

The invention provides a method (also referred to herein as a “screening assay”) for identifying modulators (e.g., small molecules, peptides, antibodies, peptidomimetics or other drugs) which bind to Pep356-related peptides, have an inhibitory or activating effect on, for example, Pep356-related peptides expression or activity, or the activity of a Pep356-related target molecule. In some embodiments small molecules can be generated using combinatorial chemistry or can be obtained from a natural products library. Assays may be cell based or non-cell based assays. Drug screening assays may be binding assays or more preferentially functional assays, as further described.

Agents that are found to increase Pep356-related peptide activity may be used, for example, to mediate angiogenesis, microfibril formation, or division of endothelial cells alone or in combination with other appropriate agents or treatments. This may be applied to cells in culture, as well as to a nonhuman or human mammal. In addition, agonists and antagonists may be applied to cultured cells or nonhuman mammals for drug development directed to: e.g., wound healing. Agonists and antagonists are also find use in therapeutic strategies directed to angiogenesis and wound healing, for example, to moderate an individual response to a selected drug.

Protein array methods are useful for screening and drug discovery. For example, one member of a receptor/ ligand pair is docked to an adsorbent, and its ability to bind the binding partner is determined in the presence of the test substance. Because of the rapidity with which adsorption can be tested, combinatorial libraries of test substances can be easily screened for their ability to modulate the interaction. In preferred screening methods, Pep356-related peptides are docked to the adsorbent. Binding partners are preferably labeled, thus enabling detection of the interaction. Alternatively, in certain embodiments, a test substance is docked to the adsorbent. The polypeptides of the invention are exposed to the test substance and screened for binding. Preferred test substances include substances correlated with a disease or disorder, such as a protein, lipid, or endocrine factor differentially present in disease.

In other embodiments, an assay is a cell-based assay in which a cell which expresses a Pep356-related peptide or biologically active portion thereof is contacted with a test compound and the ability of the test compound to inhibit, activate, or increase Pep356-related activity determined. Determining the ability of the test compound to inhibit, activate, or increase Pep356-related activity can be accomplished by monitoring the bioactivity of the Pep356-related peptide or biologically active portion thereof The cell, for example, can be of mammalian origin, insect origin, bacterial origin or a yeast cell. For example, in some embodiments, the cell can be a mammalian cell, bacterial cell or yeast cell which has been engineered to lack a natural inhibitor of a Pep356-related peptides.

In one embodiment, the invention provides assays for screening candidate compounds which are target molecules of a Pep356-related peptide or biologically active portion thereof. In another embodiment, the invention provides assays for screening candidate compounds which bind to or modulate the activity of a Pep356-related peptide or biologically active portion thereof. The test compounds of the present invention can be obtained using any of the numerous approaches in combinatorial library methods know“n in the art, including: biological libraries; spatially addressable parallel solid phase or solution phase libraries; synthetic library methods requiring deconvolution; the one-bead one-compound library method; and synthetic library methods using affinity chromatography selection. The biological library approach is used with peptide libraries, while the other four approaches are applicable to peptide, non-peptide oligomer or small molecule libraries of compounds (Lam, K. S. (1997) Anticancer Drug Des. 12:145).

Examples of methods for the synthesis of molecular libraries can be found in the art, for example in: DeWitt et al. (1993) Proc. Natl. Acad. Sci. U.S.A. 90:6909; Erb et al. (1994) Proc. Natl. Acad. Sci. USA 91:11422; Zuckermann et al. (1994). J. Med. Chem. 37:2678; Cho et al. (1993) Science 261:1303; Carrell et al. (1994) Angew. Chem. Int. Ed. Engl. 33:2059; Carell et al. (1994) Angew. Chem. Int. Ed. Engl. 33:2061; and in Gallop et al. (1994) J. Med. Chem. 37:1233.

Libraries of compounds may be presented in solution (e.g., Houghten (1992) Biotechniques 13:412-421), or on beads (Lam (1991) Nature 354:82-84), chips (Fodor (1993) Nature 364:555-556), bacteria (Ladner U.S. Pat. No. 5,223,409), spores (Ladner U.S. Pat. No. '409), plasmids (Cull et al. (1992) Proc Natl Acad Sci USA 89:1865-1869) or on phage (Scott and Smith (1990) Science 249:386-390); (Devin (1990) Science 249:404-406); (Cwirla et al. (1990) Proc. Natl. Acad. Sci. 87:6378-6382); (Felici (1991) J. Mol. Biol. 222:301-310); (Ladner supra.).

Determining the ability of the test compound to inhibit or increase Pep356-related activity can also be accomplished, for example, by coupling the Pep356-related peptide or biologically active portion thereof with a radioisotope or enzymatic label such that binding of the Pep356-related peptide or biologically active portion thereof to its cognate target molecule can be determined by detecting the labeled Pep356-related peptide or biologically active portion thereof in a complex. The labeled molecule is placed in contact with its cognate molecule and the extent of complex formation is measured. For example, the extent of complex formation may be measured by immuno precipitating the complex or by performing gel electrophoresis.

It is also within the scope of this invention to determine the ability of a compound (e.g., Pep356-related peptide or biologically active portion thereof) to interact with its cognate target molecule without the labeling of any of the interactants. For example, a microphysiometer can be used to detect the interaction of a compound with its cognate target molecule without the labeling of either the compound or the target molecule. McConnell, H. M. et al. (1992) Science 257:1906-1912. A microphysiometer such as a cytosensor is an analytical instrument that measures the rate at which a cell acidifies its environment using a light-addressable potentiometric sensor (LAPS). Changes in this acidification rate can be used as an indicator of the interaction between compound and receptor.

In a preferred embodiment, the assay comprises contacting a cell which expresses a Pep356-related peptide or biologically active portion thereof, with a target molecule to form an assay mixture, contacting the assay mixture with a test compound, and deternining the ability of the test compound to inhibit or increase the activity of the Pep356-related peptide or biologically active portion thereof, wherein determining the ability of the test compound to inhibit or increase the activity of the Pep356-related peptide or biologically active portion thereof, comprises determining the ability of the test compound to inhibit or increase a biological activity of the Pep356-related peptides expressing cell.

In another preferred embodiment, the assay comprises contacting a cell which is responsive to a Pep356-related peptide or biologically active portion thereof, with a Pep356-related peptide or biologically-active portion thereof, to form an assay mixture, contacting the assay mixture with a test compound, and determining the ability of the test compound to modulate the activity of the Pep356-related peptide or biologically active portion thereof, wherein determining the ability of the test compound to modulate the activity of the Pep356-related peptide or biologically active portion thereof comprises determining the ability of the test compound to modulate a biological activity of the Pep356-related peptides-responsive cell.

In another embodiment, an assay is a cell-based assay comprising contacting a cell expressing a Pep356-related target molecule with a test compound and determining the ability of the test compound to modulate the activity of the Pep356-related target molecule. Determining the ability of the test compound to modulate the activity of a Pep356-related target molecule can be accomplished, for example, by assessing the activity of a target molecule.

Determining the ability of the Pep356-related peptide to interact with a Pep356-related target molecule, for example a natural Pep356-related inhibitor, can be accomplished by one of the methods described for determining direct binding. In a preferred embodiment, determining the ability of the Pep356-related peptide to interact with a Pep356-related target molecule can be accomplished by determining the activity of the target molecule. For example, the activity of the target molecule can be determined by contacting the target molecule with the Pep356-related peptide or a fragment thereof and measuring induction of a cellular second messenger of the target (i.e. intracellular Ca2+, diacylglycerol, IP3, etc.), detecting catalytic/enzymatic activity of the target on appropriate substrate, detecting the induction of a reporter gene (comprising a target-responsive regulatory element operatively linked to a nucleic acid encoding a detectable marker, e.g., luciferase), or detecting a target-regulated cellular response, for example, signal transduction or protein:protein interactions.

In yet another embodiment, an assay of the present invention is a cell-free assay in which a Pep356-related peptide or biologically active portion thereof is contacted with a test compound and the ability of the test compound to bind to the Pep356-related peptide or biologically active portion thereof is determined. Binding of the test compound to the Pep356-related peptide can be determined either directly or indirectly as described above. In a preferred embodiment, the assay includes contacting the Pep356-related peptide or biologically active portion thereof with a known compound which binds Pep356-related peptides (e.g., a Pep356-related target molecule) to form an assay mixture, contacting the assay mixture with a test compound, and determining the ability of the test compound to interact with a Pep356-related peptide, wherein determining the ability of the test compound to interact with a Pep356-related peptide comprises determining the ability of the test compound to preferentially bind to Pep356-related peptides or biologically active portion thereof as compared to the known compound.

In another embodiment, the assay is a cell-free assay in which a Pep356-related peptide or biologically active portion thereof is contacted with a test compound and the ability of the test compound to modulate the activity of the Pep356-related peptide or biologically active portion thereof is detennined. Determining the ability of the test compound to modulate the activity of a Pep356-related peptide can be accomplished, for example, by determining the ability of the Pep356-related peptide to bind directly to a Pep356-related target molecule or by using a technology such as real-time Biomolecular Interaction Analysis (BIA). Sjolander, S. and Urbaniczky, C. (1991) Anal. Chem. 63:2338-2345 and Szabo et al. (1995) Curr. Opin. Struct. Biol. 5:699-705. As used herein, “BIA” is a technology for studying biospecific interactions in real time, without labeling any of the interactants (e.g., BIAcore). Changes in the optical phenomenon of surface plasmon resonance (SPR) can be used as an indication of real-time reactions between biological molecules.

In an alternative embodiment, determining the ability of the test compound to modulate the activity of a Pep356-related peptide can be accomplished by determining the ability of the Pep356-related peptide to further modulate the activity of a downstream effector (e.g., a growth factor mediated signal transduction pathway component) of a Pep356-related target molecule. For example, the activity of the effector molecule on an appropriate target can be determined or the binding of the effector to an appropriate target can be determined as previously described.

In yet another embodiment, the cell-free assay involves contacting a Pep356-related peptide or biologically active portion thereof with a known compound which binds the Pep356-related peptide to formn an assay mixture, contacting the assay mixture with a test compound, and determining the ability of the test compound to interact with the Pep356-related peptide, wherein determining the ability of the test compound to interact with the Pep356-related peptide comprises determining the ability of the Pep356-related peptide to preferentially bind to or modulate the activity of a Pep356-related target molecule.

The cell-free assays of the present invention are amenable to use of both soluble and/or membrane-bound forms of isolated proteins (e.g. Pep356-related peptides or biologically active portions thereof or molecules to which Pep356-related peptides targets bind). In the case of cell-free assays in which a membrane-bound form an isolated protein is used it may be desirable to utilize a solubilizing agent such that the membrane-bound form of the isolated protein is maintained in solution. Examples of such solubilizing agents include non-ionic detergents such as n-octylglucoside, n-dodecylglucoside, n-dodecylmaltoside, octanoyl-N-methylglucamide, decanoyl-N-methylglucamide, Triton™ X-100, Triton™ X-114, Thesit™, Isotridecypoly(ethylene glycol ether)n,3-[(3-cholamidopropyl)dimethylamminio]-1-propane sulfonate (CHAPS), 3-[(3-cholamidopropyl)dimethylamminio]-2-hydroxy-1-propane sulfonate (CHAPSO), or N-dodecyl=N,N-dimethyl-3-ammonio-1-propane sulfonate.

In more than one embodiment of the above assay methods, it may be desirable to immobilize either a Pep356-related peptide or its target molecule to facilitate separation of complexed from uncomplexed forms of one or both of the proteins, as well as to accommodate automation of the assay. Binding of a test compound to a Pep356-related peptide, or interaction of a Pep356-related peptide with a target molecule in the presence and absence of a candidate compound, can be accomplished in any vessel suitable for containing the reactants and by any imunobilization protocol described herein. Alternatively, the complexes can be dissociated from the matrix, and the level of Pep356-related peptides binding or activity determined using standard techniques.

Other techniques for immnobilizing proteins on matrices can also be used in the screening assays of the invention. For example, either a Pep356-related peptide or a Pep356-related target molecule can be immobilized utilizing conjugation of biotin and streptavidin. Biotinylated Pep356-related peptide or target molecules can be prepared from biotin-NHS (N-hydroxy-succinimide) using techniques well known in the art (e.g., biotinylation kit, Pierce Chemicals, Rockford, Ill.), and immobilized in the wells of streptavidin-coated 96 well plates (Pierce Chemical). Alternatively, antibodies reactive with Pep356-related peptide or target molecules but which do not interfere with binding of the Pep356-related peptide to its target molecule can be derivatized to the wells of the plate, and unbound target or Pep356-related peptide trapped in the wells by antibody conjugation. Methods for detecting such complexes, in addition to those described above for the GST-immobilized complexes, include immunodetection of complexes using antibodies reactive with the Pep356-related peptide or target molecule, as well as enzyme-linked assays which rely on detecting an enzymatic activity associated with the Pep356-related peptide or target molecule.

In another embodiment, modulators of Pep356-related peptides expression are identified in a method wherein a cell is contacted with a candidate compound and the expression of Pep356-related peptides mRNA or protein in the cell is deternined. The level of expression of Pep356-related peptides mRNA or protein in the presence of the candidate compound is compared to the level of expression of Pep356-related peptides mRNA or protein in the absence of the candidate compound. The candidate compound can then be identified as a modulator of Pep356-related peptides expression based on this comparison. For example, when expression of Pep356-related peptides mRNA or protein is greater (statistically significantly greater) in the presence of the candidate compound than in its absence, the candidate compound is identified as a stimulator of Pep356-related peptides mRNA or protein expression. Alternatively, when expression of Pep356-related peptides mRNA or protein is less (statistically significantly less) in the presence of the candidate compound than in its absence, the candidate compound is identified as an inhibitor of Pep356-related peptides mRNA or protein expression. The level of Pep356-related peptides mRNA or protein expression in the cells can be determined by methods described herein for detecting Pep356-related peptides mRNA or protein.

In yet another aspect of the invention, the Pep356-related peptides can be used as “bait proteins” in a two-hybrid assay or three-hybrid assay (see, e.g., U.S. Pat. No. 5,283,317; Zervos et al. (1993) Cell 72:223-232; Madura et al. (1993) J. Biol. Chem. 268:12046-12054; Bartel et al. (1993) Biotechniques 14:920-924; Iwabuchi et al. (1993) Oncogene 8:1693-1696; and Brent WO94/10360), to identify other proteins, which bind to or interact with Pep356-related peptides (“Pep356-related peptides-binding proteins” or “Pep356-related peptides-bp”) and are involved in Pep356-related activity. Such Pep356-related peptides-binding proteins are also likely to be involved in the propagation of signals by the Pep356-related peptides or Pep356-related peptides targets as, for example, downstream elements of a Pep356-related peptides-mediated signaling pathway. Alternatively, such Pep356-related peptides-binding proteins are likely to be Pep356-related inhibitors.

The two-hybrid system is based on the modular nature of most transcription factors, which consist of separable DNA-binding and activation domains. Briefly, the assay utilizes two different DNA constructs. In one construct, the gene that codes for a Pep356-related peptide or a fragment thereof is fused to a gene encoding the DNA binding domain of a known transcription factor (e.g., GAL-4). In the other construct, a DNA sequence, from a library of DNA sequences, that encodes an unidentified protein (“prey” or “sample”) is fused to a gene that codes for the activation domain of the known transcription factor. If the “bait” and the “prey” proteins are able to interact, in vivo, forming a Pep356-related peptides-dependent complex, the DNA-binding and activation domains of the transcription factor are brought into close proximity. This proximity allows transcription of a reporter gene (e.g., LacZ) which is operably linked to a transcriptional regulatory site responsive to the transcription factor. Expression of the reporter gene can be detected and cell colonies containing the functional transcription factor can be isolated and used to obtain the cloned gene which encodes the protein which interacts with the Pep356-related peptide.

This invention further pertains to novel agents identified by the above-described screening assays and to processes for producing such agents by use of these assays. Accordingly, in one embodiment, the present invention includes a compound or agent obtainable by a method comprising the steps of any one of the aforementioned screening assays (e.g., cell-based assays or cell-free assays). Accordingly, it is within the scope of this invention to flirther use an agent identified in a described screening assay in an appropriate animal model. For example, an agent identified as described herein (e.g., a Pep356-related peptides modulating agent) can be used in an animal model to determine the efficacy, toxicity, or side effects of treatment with such an agent. Alternatively, an agent identified as described herein can be used in an animal model to determine the mechanism of action of such an agent. Furthermore, this invention pertains to uses of novel agents identified by the above-described screening assays for treatments as described herein.

The present inventon also pertains to uses of novel agents identified by the above-described screening assays for diagnoses, prognoses, and treatments as described herein. Accordingly, it is within the scope of the present invention to use such agents in the design, formulation, synthesis, manufacture, and/or production of a drug or pharmaceutical composition for use in diagnosis, prognosis, or treatment, as described herein. For example, in one embodiment, the present invention includes a method of synthesizing or producing a drug or pharmaceutical composition by reference to the structure and/or properties of a compound obtainable by one of the above-described screening assays.

Pharmaceutical Compositions

When polypeptides of the present invention are expressed in soluble form, for example as a secreted product of transformed yeast or marnrnalian cells, they can be purified according to standard procedures of the art, including steps of ammonium sulfate precipitation, ion exchange chromatography, gel filtration, electrophoresis, affinity chromatography, and/or the like, e.g. “Enzyme Purification and Related Techniques,” Methods in Enzymology, 22:233-577 (1977), and Scopes, R., Protein Purification: Principles and Practice (Springer-Verlag, New York, 1982) provide guidance in such purifications. Likewise, when polypeptides of the invention are expressed in insoluble form, for example as inclusion bodies, they can be purified by standard procedures in the art, including separating the inclusion bodies from disrupted host cells by centrifugation, solubilizing the inclusion bodies with chaotropic and reducing agents, diluting the solubilized mixture, and lowering the concentration of chaotropic agent and reducing agent so that the polypeptide takes on a biologically active conformation, The latter procedures are disclosed in the following references: Winkler et al, Biochemistry, 25: 40414045 (1986); Winkler et al, Biotechnology, 3: 992-998 (1985); Koths et al, U.S. Pat. No. 4,569,790; and European patent applications 86306917.5 and 86306353.3.

Compounds capable of inhibiting a Pep356-related activity, preferably small molecules, may be incorporated into pharmaceutical compositions suitable for administration. Such compositions typically comprise a pharmaceutically acceptable carrier. As used herein the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

A pharmaceutical composition of the invention is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL® (BASF, Parsippany, N.J.) or phosphate buffered saline (PBS). In all cases, the composition must be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Where the active compound is a protein, e.g., a Pep356-related peptide or anti-Pep356-related antibody, sterile injectable solutions can be prepared by incorporating the active compound in the required amount of an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freezecying which yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. For administration by inhalation, the compounds are delivered in the form of an aerosol spray from pressured container or dispenser which contains a suitable propellant, e.g., a gas such as carbon dioxide, or a nebulizer. Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art. Most preferably, active compound is delivered to a subject by intravenous injection.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared as described in U.S. Pat. No. 4,522,811.

In a further embodiment, the active compound (e.g., a Pep356-related peptide) may be coated on a microchip drug delivery device. Such devices are useful for controlled delivery of proteinaceous compositions into the bloodstream, cerebrospinal fluid, lymph, or tissue of an individual without subjecting such compositions to digestion or subjecting the individual to injection. Methods of using microchip drug delivery devices are described in U.S. Pat. Nos. 6,123,861 and 5,797,898 and US Patent application 20020119176A1.

It is especially advantageous to formulate oral or preferably parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

Toxicity and therapeutic efficacy of such compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds with large therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC50 (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

The pharmaceutical compositions can be included in a container, pack, or dispenser together with instructions for administration.

References cited herein are incorporated in their entireties. Having generally described this invention, a further understanding can be obtained by reference to certain specific examples which are provided herein for purposes of illustration only, and are not intended to be limiting unless otherwise specified.

### EXAMPLES

### Example 1

### Purification and Characterisation of a Pep356-related Peptide

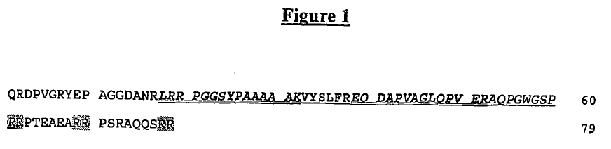

Human plasma was fractionated by a series of multiple chromatography columns. The fractions were analysed by mass spectrometry. Five tryptic fragments were found to belong to the protein sequence of FIG. 1 by tandem and MALDI mass spectrometry: LRRPGGSYPAAAAAK, RPGGSYPAAAAAK, VYSLFREQDAPVAGLQPVER, EQDAPVAGLQPVER and AQPGWGSPR.

The Pep356-related peptides were found to possess sequence identity with a sub-region of a latent transforming growth factor binding protein (accession number Q14767, available at http://www.expasy.ch, and Moren A., et al., J. Biol. Chem. 269:32469-32478(1994)). The peptides of SEQ ID NOs: 3 to 5 each correspond to a cleavage product at a different dibasic site of the mature form of the LTBP2 protein. The mature form of the LTBP2 protein is obtained from the precusor by cleavage of the signal peptide after position 35 (GHA-QR). SEQ ID NO: 1 and 2 represent the precursor and mature LTBP2 protein, respectively.

### Example 2

### Chemical Synthesis of a Pep356-related Peptide

In this example, a Pep356-related peptide of the invention is synthesized. Peptide fragment intermediates are first synthesized and then assembled into the desired polypeptide.

A Pep356-related peptide can initially be. prepared in, e.g. 5 fragments, selected to have a Cys residue at the N-terminus of the fragment to be coupled. Fragment 1 is initially coupled to fragment 2 to give a first product, then after preparative HPLC purification, the first product is coupled to fragment 3 to give a second product. After preparative HPLC purification, the second product is coupled to fragment 4 to give a third product. Finally, after preparative HPLC purification, the third product is coupled to fragment 5 to give the desired polypeptide, which is purified and refolded.

Thioester formation. Fragments 2, 3, 4, and 5 are synthesized on a thioester generating resin, as described above. For this purpose the following resin is prepared: S-acetylthioglycolic acid pentafluorophenylester is coupled to a Leu-PAM resin under conditions essentially as described by Hackeng et al (1999). In the first case, the resulting resin is used as a starting resin for peptide chain elongation on a 0.2 mmol scale after removal of the acetyl protecting group with a 30 min treatment with 10% mercaptoethanol, 10% piperidine in DMF. The N^{α} of the N-terminal Cys residues of fragments 2 through 5 are protected by coupling a Boc-thioproline (Boc-SPr, i.e. Boc-L-thioproline) to the terminus of the respective chains instead of a Cys having conventional N^{α} or S^{β} protection, e.g. Brik et al, J. Org. Chem., 65: 3829-3835 (2000).

Peptide synthesis. Solid-phase synthesis is performed on a custom-modified 433A peptide synthesizer from Applied Biosystems, using in situ neutralization/2-(1H-benzotriazol-1-yl)-1,1,1,3,3-tetramethyluronium hexafluoro-phosphate (DEBTS) activation protocols for stepwise Boc chemistry chain elongation, as described by Schnolzer et al, Int. J. Peptide Protein Res., 40: 180-193 (1992). Each synthetic cycle consists of N^{α}-Boc-removal by a 1 to 2 min treatment with neat TFA, a 1-min DMF flow wash, a 10-min coupling time with 2.0 mmol of preactivated Boc-amino acid in the presence of excess DIEA and a second DMF flow wash. Nct-Boc-amino acids (2 mmol) are preactivated for 3 min with 1.8 mmol HBTU (0.5M in DMF) in the presence of excess DIEA (6 mmol). After coupling of Gln residues, a dichloromnethane flow wash is used before and after deprotection using TFA, to prevent possible high temperature (TFA/DMF)-catalyzed pyrrolidone carboxylic acid formation. Side-chain protected amino acids are Boc-Arg(p-toluenesulfonyl)-OH, Boc-Asn(xanthyl)-OH, Boc-Asp(O-cyclohexyl)-OH, Boc-Cys(4-methylbenzyl)-OH, Boc-Glu(O-cyclohexyl)-OH, Boc-His(dinitrophenylbenzyl)-OH, Boc-Lys(2-Cl-Z)-OH, Boc-Ser(benzyl)-OH, Boc-Thr(benzyl)-OH, Boc-Trp(formyl)-OH and Boc-Tyr(2-Br-Z)-OH (Orpagen Pharmna, Heidelberg, Germany). Other amino acids are used without side chain protection. C-terminal Fragment 1 is synthesized on Boc-Leu-O-CH₂-Pam resin (0.71 mmol/g of loaded resin), while for Fragments 2 through 5 machine-assisted synthesis is started on the Boc-Xaa-S-CH₂-CO-Leu-Pam resin. This resin is obtained by the coupling of S-acetylthioglycolic acid pentafluorophenylester to a Leu-PAM resin under standard conditions. The resulting resin is used as a starting resin for peptide chain elongation on a 0.2 mmol scale after removal of the acetyl protecting group with a 30min treatment with 10% mercaptoethanol, 10% piperidine in DMF.

After chain assembly is completed, the peptide fragments are deprotected and cleaved from the resin by treatment with anhydrous hydrogen fluoride for 1 hr at 0° C. with 5% p-cresol as a scavenger. In all cases except Fragment 1, the imidazole side chain 2,4-dinitrophenyl (DNP) protecting groups remain on His residues because the DNP-removal procedure is incompatible with C-terminal thioester groups. However DNP is gradually removed by thiols during the ligation reaction, yielding unprotected His. After cleavage, peptide fragrnents are precipitated with ice-cold diethylether, dissolved in aqueous acetonitrile and lyophilized. The peptide fragments are purified by RP-HPLC with a C18 column from Waters by using linear gradients of buffer B (acetonitile/0.1% trifluoroacetic acid) in buffer A (H₂O/0.1% trifluoroacetic acid) and UV detection at 214 nm. Samples are analyzed by electrospray mass spectrometry (ESMS) using an Esquire instrument (Brucker, Bremen, Germany), or like instrument.

Native chernical ligations. As described more fully below, the ligation of unjprotected fragments is performed as follows: the dry peptides are dissolved in equimolar amounts in 6M guanidine hydrochloride (GuHCl), 0.2M phosphate, pH 7.5 in order to get a final peptide concentration of 1-8 mM at a pH around 7, and 1% benzylmercaptan, 1% thiophenol is added. Usually, the reaction is carried out overnight and is monitored by HPLC and electrospray mass spectrometry. The ligation product is subsequently treated to remove protecting groups still present. The formyl group of Trp is cleaved by shifting the pH of the solution up to 9.0 with hydrazine and incubating for 1 h at 37° C. Opening of the N-terminal thiazolidine ring further required the addition of solid methoxamine to a 0.5M final concentration at pH3.5 and a further incubation for 2 h at 37° C. A 10-fold excess of Tris(2-carboxyethyl)phosphine is added before preparative HPLC purification. Fractions containing the polypeptide chain are identified by ESMS, pooled and lyophilized.

The ligation of fragments 4 and 5 is performed at pH7.0 in 6 M GuHCl. The concentration of each reactant is 8 mM, and-1% benzylmercaptan and 1% thiophenol were added to create a reducing environment and to facilitate the ligation reaction. An almost quantitative ligation reaction is observed after overnight stirring at 37° C. At this point in the reaction, O-NH₂.HCl is added as a powder to a 0.1 M final concentration and hydrazine is added to shift the pH to 9.0, for the removal of the formyl group of any Trp residues. After a 1 h incubation at 37° C, O—NH₂.HCl is further added to the solution to get a 0.5M final concentration, and the pH adjusted to 3.5 in order to open the N-terminal thiazolidine ring. After 2 h incubation at 37° C., ESMS is used to confirm the completion of the reaction. The reaction mixture is subsequently treated with a 10-fold excess of Tris(2-carboxyethylphosphine) over the peptide fragment and after 15 min, the ligation product is purified using the preparative HPLC (e.g., C4, 20-60% CH₃CN, 0.5% per min), lyophilised, and stored at −20° C.

The same procedure is repeated for the remaining ligations with slight modification. Whenever the ligation takes place at an Ile-Cys or Val-Cys site, the ligation reaction is extended to 48 h.

Polypeptide Folding. The full length peptide is refolded by air oxidation by dissolving the reduced lyophilized protein (about 0.1 mg/mL) in 1 M GuHCl, I OOmM Tris, 10 mM methionine, pH 8.6 After gentle stirring overnight, the protein solution is purified by RP-HPLC as described above.

### Example 3

### Preparation of Antibody Compositions

Substantially pure Pep356-related peptide or a portion thereof is obtained. The concentration of protein in the final preparation is adjusted, for example, by concentration on an Amicon filter device, to the level of a few micrograms per ml. Monoclonal or polyclonal antibodies to the protein are then prepared as described in the sections titled “Monoclonal antibodies” and “Polyclonal antibodies.”

Briefly, to produce an anti-Pep356 related peptide monoclonal antibody, a mouse is repetitively inoculated with a few micrograms of the Pep356-related peptide or a portion thereof over a period of a few weeks. The mouse is then sacrificed, and the antibody producing cells of the spleen isolated. The spleen cells are fused by means of polyethylene glycol with mouse myeloma cells, and the excess unfused cells destroyed by growth of the system on selective media comprising aminopterin (HAT media). The successfully fused cells ate diluted and aliquots of the dilution placed in wells of a microtiter plate where growth of the culture is continued. Antibody-producing clones are identified by detection of antibody in the supernatant fluid of the wells by immunoassay procedures, such as ELISA, as originally described by Engvall, E., Meth. Enzymol. 70: 419 (1980). Selected positive clones can be expanded and their monoclonal antibody product harvested for use. Detailed procedures for monoclonal antibody production are described in Davis, L. et al. Basic Methods in Molecular Biology Elsevier, New York. Section 21-2.

For polyclonal antibody production by iinuunization, polyclonal antiserum containing antibodies to heterogeneous epitopes in the Pep356-related peptide or a portion thereof are prepared by immunizing a mouse with the Pep356-related peptide or a portion thereof, which can be unmodified or modified to enhance immunogenicity. Any suitable nonhuman animal, preferably a non-human mammal, may be selected including rat, rabbit, goat, or horse.

Antibody preparations prepared according to either the monoclonal or the polyclonal protocol are useful in quantitative immunoassays which determine concentrations of Pep356-related peptide in biological samples; or they are also used semni-quantitatively or qualitatively to identify the presence of antigen in a biological sample. The antibodies may also be used in therapeutic compositions for killing cells expressing the protein or reducing the levels of the protein in the body.

1. An isolated polypeptide comprising an amino acid sequence selected from the group consisting of SEQ ID Nos. 3-5, wherein said polypeptide has biological activity.

2. The polypeptide of claim 1, wherein said polypeptide is fused to a heterologous polypeptide sequence.

3. A method of making a Pep356-related peptide, said method comprising the steps of:
i) providing a population of host cells capable of expressing the polypeptide of claim 1;
ii) culturing said population of host cells under conditions conducive to the expression of said polypeptide;
iii) isolating said polypeptide.

4. A Pep356-related peptide antibody that selectively binds to the polypeptide of claim 1.

5. A method of binding an antibody to a protein comprising the steps of:
i) contacting an antibody that selectively binds the polypeptide of claim 1 with said polypeptide; and
ii) removing nonbinding contaminants.

6. A composition comprising the polypeptide according to claim 1, further comprising a carrier or diluent.

7. A method for modulating microfibril structure, comprising the step of contacting a polypeptide of claim 1 to a population of cells.

8. The method of claim 7, wherein said composition is administered to an individual.

9. The method of claim 8, wherein said composition is administered by injection.

10. A method of identifying a candidate Pep356-related peptide modulator, said method comprising:
(a) contacting said Pep356-related peptide of claim 1 with a test compound; and
(b) determining whether said compound selectively modulates a biological activity of said Pep356-related peptide, wherein a determination that said compound modulates a Pep356-related activity indicates that said compound is a candidate Pep356-related peptide modulator.


SEQUENCE LISTING (10 sequences)

SEQ ID NO: 1 - protein, 1821 aa, Homo sapiens
  PROPEP (1)...(1821): Latent transforming growth factor-beta-binding protein precursor 2
MRPRTKARSPGRALRNPWRGFLPLTLALFVGAGHAQRDPVGRYEPAGGDANRLRRPGGSYPAAAAAKVYSLFREQDAPVAGLQPVERAQPGWGSPRRPTEAEARRPSRAQQSRRVQPPAQTRRSTPLGQQQPAPRTRAAPALPRLGTPQRSGAAPPTPPRGRLTGRNVCGGQCCPGWTTANSTNHCIKPVCEPPCQNRGSCSRPQLCVCRSGFRGARCEEVIPDEEFDPQNSRLAPRRWAERSPNLRRSSAAGEGTLARAQPPAPQSPPAPQSPPAGTLSGLSQTHPSQQHVGLSRTVRLHPTATASSQLSSNALPPGPGLEQRDGTQQAVPLEHPSSPWGLNLTEKIKKIKIVFTPTICKQTCARGHCANSCERGDTTTLYSQGGHGHDPKSGFRIYFCQIPCLNGGRCIGRDECWCPANSTGKFCHLPIPQPDREPPGRGSRPRALLEAPLKQSTFTLPLSNQLASVNPSLVKVHIHHPPEASVQIHQVAQVRGGVEEALVENSVETRPPPWLPASPGHSLWDSNNIPARSGEPPRPLPPAAPRPRGLLGRCYLNTVNGQCANPLLELTTQEDCCGSVGAFWGVTLCAPCPPRPASPVIENGQLECPQGYKRLNLTHCQDINECLTLGLCKDAECVNTRGSYLCTCRPGLMLDPSRSRCVSDKAISMLQGLCYRSLGPGTCTLPLAQRITKQICCCSRVGKAWGSECEKCPLPGTEAFREICPAGHGYTYASSDIRLSMRKAEEEELARPPREQGQRSSGALPGPAERQPLRVVTDTWLEAGTIPDKGDSQAGQVTTSVTHAPAWVTGNATTPPMPEQGIAEIQEEQVTPSTDVLVTLSTPGIDRCAAGATNVCGPGTCVNLPDGYRCVCSPGYQLHPSQAYCTDDNECLRDPCKGKGRCINRVGSYSCFCYPGYTLATSGATQECQDINECEQPGVCSGGQCTNTEGSYHCECDQGYIMVRKGHCQDINECRHPGTCPDGRCVNSPGSYTCLACEEGYRGQSGSCVDVNECLTPGVCAHGKCTNLEGSFRCSCEQGYEVTSDEKGCQDVDECASRASCPTGLCLNTEGSFACSACENGYWVNEDGTACEDLDECAFPGVCPSGVCTNTAGSFSCKDCDGGYRPSPLGDSCEDVDECEDPQSSCLGGECKNTVGSYQCLCPQGFQLANGTVCEDVNECMGEEHCAPHGECLNSHGSFFCLCAPGFVSAEGGTSCQDVDECATTDPCVGGHCVNTEGSFNCLCETGFQPSPESGECVDIDECEDYGDPVCGTWKCENSPGSYRCVLGCQPGFHMAPNGDCIDIDECANDTMCGSHGFCDNTDGSFRCLCDQGFEISPSGWDCVDVNECELMLAVCGAALCENVEGSFLCLCASDLEEYDAQEGHCRPRGAGGQSMSEAPTGDHAPAPTRMDCYSGQKGHAPCSSVLGRNTTQAECCCTQGASWGDACDLCPSEDSAEFSEICPSGKGYIPVEGAWTFGQTMYTDADECVIFGPGLCPNGRCLNTVPGYVCLCNPGFHYDASHKKCEDHDECQDLACENGECVNTEGSFHCFCSPPLTLDLSQQRCMNSTSSTEDLPDHDIHMDICWKKVTNDVCSEPLRGHRTTYTECCCQDGEAWSQQCALCPPRSSEVYAQLCNVARIEAEREAGVHFRPGYEYGPGPDDLHYSIYGPDGAPFYNYLGPEDTVPEPAFPNTAGHSADRTPILESPLQPSELQPHYVASHPEPPAGFEGLQAEECGILNGCENGRCVRVREGYTCDCFEGFQLDAAHMACVDVNECDDLNGPAVLCVHGYCENTEGSYRCHCSPGYVAEAGPPHCTAKE

SEQ ID NO: 2 - protein, 1786 aa, Homo sapiens
QRDPVGRYEPAGGDANRLRRPGGSYPAAAAAKVYSLFREQDAPVAGLQPVERAQPGWGSPRRPTEAEARRPSRAQQSRRVQPPAQTRRSTPLGQQQPAPRTRAAPALPRLGTPQRSGAAPPTPPRGRLTGRNVCGGQCCPGWTTANSTNHCIKPVCEPPCQNRGSCSRPQLCVCRSGFRGARCEEVIPDEEFDPQNSRLAPRRWAERSPNLRRSSAAGEGTLARAQPPAPQSPPAPQSPPAGTLSGLSQTHPSQQHVGLSRTVRLHPTATASSQLSSNALPPGPGLEQRDGTQQAVPLEHPSSPWGLNLTEKIKKIKIVFTPTICKQTCARGHCANSCERGDTTTLYSQGGHGHDPKSGFRIYFCQIPCLNGGRCIGRDECWCPANSTGKFCHLPIPQPDREPPGRGSRPRALLEAPLKQSTFTLPLSNQLASVNPSLVKVHIHHPPEASVQIHQVAQVRGGVEEALVENSVETRPPPWLPASPGHSLWDSNNIPARSGEPPRPLPPAAPRPRGLLGRCYLNTVNGQCANPLLELTTQEDCCGSVGAFWGVTLCAPCPPRPASPVIENGQLECPQGYKRLNLTHCQDINECLTLGLCKDAECVNTRGSYLCTCRPGLMLDPSRSRCVSDKAISMLQGLCYRSLGPGTCTLPLAQRITKQICCCSRVGKAWGSECEKCPLPGTEAFREICPAGHGYTYASSDIRLSMRKAEEEELARPPREQGQRSSGALPGPAERQPLRVVTDTWLEAGTIPDKGDSQAGQVTTSVTHAPAWVTGNATTPPMPEQGIAEIQEEQVTPSTDVLVTLSTPGIDRCAAGATNVCGPGTCVNLPDGYRCVCSPGYQLHPSQAYCTDDNECLRDPCKGKGRCINRVGSYSCFCYPGYTLATSGATQECQDINECEQPGVCSGGQCTNTEGSYHCECDQGYIMVRKGHCQDINECRHPGTCPDGRCVNSPGSYTCLACEEGYRGQSGSCVDVNECLTPGVCAHGKCTNLEGSFRCSCEQGYEVTSDEKGCQDVDECASRASCPTGLCLNTEGSFACSACENGYWVNEDGTACEDLDECAFPGVCPSGVCTNTAGSFSCKDCDGGYRPSPLGDSCEDVDECEDPQSSCLGGECKNTVGSYQCLCPQGFQLANGTVCEDVNECMGEEHCAPHGECLNSHGSFFCLCAPGFVSAEGGTSCQDVDECATTDPCVGGHCVNTEGSFNCLCETGFQPSPESGECVDIDECEDYGDPVCGTWKCENSPGSYRCVLGCQPGFHMAPNGDCIDIDECANDTMCGSHGFCDNTDGSFRCLCDQGFEISPSGWDCVDVNECELMLAVCGAALCENVEGSFLCLCASDLEEYDAQEGHCRPRGAGGQSMSEAPTGDHAPAPTRMDCYSGQKGHAPCSSVLGRNTTQAECCCTQGASWGDACDLCPSEDSAEFSEICPSGKGYIPVEGAWTFGQTMYTDADECVIFGPGLCPNGRCLNTVPGYVCLCNPGFHYDASHKKCEDHDECQDLACENGECVNTEGSFHCFCSPPLTLDLSQQRCMNSTSSTEDLPDHDIHMDICWKKVTNDVCSEPLRGHRTTYTECCCQDGEAWSQQCALCPPRSSEVYAQLCNVARIEAEREAGVHFRPGYEYGPGPDDLHYSIYGPDGAPFYNYLGPEDTVPEPAFPNTAGHSADRTPILESPLQPSELQPHYVASHPEPPAGFEGLQAEECGILNGCENGRCVRVREGYTCDCFEGFQLDAAHMACVDVNECDDLNGPAVLCVHGYCENTEGSYRCHCSPGYVAEAGPPHCTAKE

SEQ ID NO: 3 - protein, 79 aa, Homo sapiens
QRDPVGRYEPAGGDANRLRRPGGSYPAAAAAKVYSLFREQDAPVAGLQPVERAQPGWGSPRRPTEAEARRPSRAQQSRR

SEQ ID NO: 4 - protein, 70 aa, Homo sapiens
QRDPVGRYEPAGGDANRLRRPGGSYPAAAAAKVYSLFREQDAPVAGLQPVERAQPGWGSPRRPTEAEARR

SEQ ID NO: 5 - protein, 79 aa, Homo sapiens; identical to SEQ ID NO: 3

SEQ ID NO: 6 - protein, 45 aa, Homo sapiens
LRRPGGSYPAAAAAKVYSLFREQDAPVAGLQPVERAQPGWGSPRR

SEQ ID NO: 7 - protein, 13 aa, Homo sapiens
RPGGSYPAAAAAK

SEQ ID NO: 8 - protein, 20 aa, Homo sapiens
VYSLFREQDAPVAGLQPVER

SEQ ID NO: 9 - protein, 14 aa, Homo sapiens
EQDAPVAGLQPVER

SEQ ID NO: 10 - protein, 9 aa, Homo sapiens
AQPGWGSPR